SEQUENCE LISTING (118 sequences)

SEQ ID NO: 1 - protein, 323 aa, Zea mays
MSAAPAAGGQGGGGMDAALLDDIIRRLLEVRTARPGKQVQLSESEIRQLCTVSREIFLNQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLAHLDEIKNLQRPTDVPDQGLLCDLLWSDPGKDVQGWGMNDRGVSYTFGADKVSEFLQRHDLDLICRAHQVVEDGYEFFADRQLVTIFSAPNYCGEFDNAGAMMSVDETLMCSFQILKPAERKQIYGAKQNVR

SEQ ID NO: 2 - protein, 322 aa, Zea mays
MAAAPAAGGQGGGGMDAALLDDIIRRLLEVRTARPGKQVQLSESEIRQLCTVSREIFLNQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLAHLDEIKNLQRPTDVPDQGLLCDLLWSDPGKDAQGWGMNDRGVSYTFGADKVSEFLQKHDLDLICRAHQVVEDGYEFFADRQLVTIFSAPNYCGEFDNAGAMMSVDETLMCSFQILKPAERKNKFMGSNKM

SEQ ID NO: 3 - protein, 323 aa, Zea mays
MSAAPAAGGQGGGGIDAALLDDIIRRLLEVRTARPGKQVQLSESEIRQLCTVSRAIFLSQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLAHLDEIKNLQRPTDVPDQGLLCDLLWSDPGKDVQGWGMNDRGVSYTFGADKVSEFLQRHDLDLICRAHQVVEDGYEFFADRQLVTIFSAPNYCGEFDNAGAMMSVDETLMCSFQILKPAERKQIYGAKQNVR

SEQ ID NO: 4 - protein, 322 aa, Sorghum bicolor
MAAAPAAGGQGGVGMDTALVDDIIRRLLEVRTARPGKQVQLSESEIRQLCNVSREIFLSQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLAHLDEIKSLQRPTDVPDQGLLCDLLWSDPGKDVQGWGMNDRGVSYTFGADKVSEFLQKHDLDLICRAHQVVEDGYEFFADRQLVTIFSAPNYCGEFDNAGAMMSVDETLMCSFQILKPAERKNKFMGSNKM

SEQ ID NO: 5 - protein, 322 aa, Oryza sativa
MAAAPGAGGQGGGGMDAVLLDDIIRRLLEVRTARPGKQVQLSESEIRQLCTVSREIFLSQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTDCFNCLPVAALIDDKILCMHGGLSPDLTHLDEIKSLPRPTDVPDTGLLCDLLWSDPGKDVQGWGMNDRGVSYTFGADKVSEFLEKHDLDLICRAHQVVEDGYEFFADRQLVTIFSAPNYCGEFDNAGAMMSVDETLMCSFQILKPAERKGKFMASNKM

SEQ ID NO: 6 - protein, 321 aa, Arabidopsis thaliana
MATTTTTQGQQTAIDSAVLDDIIRRLTEVRLARPGKQVQLSEAEIKQLCTTARDIFLQQPNLLELEAPIKICGDIHGQYSDLLRLFEYGGFPPSANYLFLGDYVDRGKQSLETICLLLAYKIKYPGNFFLLRGNHECASINRIYGFYDECKRRFNVRVWKVFTDCFNCLPVAALIDDKILCMHGGLSPDLDHLDEIRNLPRPTMIPDTGLLCDLLWSDPGKDVKGWGMNDRGVSYTFGPDKVSEFLTKHDLDLVCRAHQVVEDGYEFFADRQLVTVFSAPNYCGEFDNAGAMMSVDENLMCSFQILKPAEKKTKFMMSTKI

SEQ ID NO: 7 - protein, 316 aa, Glycine max
MSTQGQVIIDEAVLDDIIRRLTEVRLARPGKQVQLSESEIKQLCVASRDIFINQPNLLELEAPIKICGDIHGQYSDLLRLFEYGGLPPTANYLFLGDYVDRGKQSLETICLLLAYKIKYPENFFLLRGNHECASINRIYGFYDECKRRFNVRLWKAFTDCFNFLPVAALIDDKILCMHGGLSPELTNLDEIRNLPRPTAIPDTGLLCDLLWSDPGRDVKGWGMNDRGVSYTFGPDKVAEFLTKHDLDLICRAHQVVEDGYEFFADRQLVTIFSAPNYCGEFDNAGAMMSVDENLMCSFQILKPAEKKSKFVMSNKM

SEQ ID NO: 8 - protein, 315 aa, Glycine max
MSTQGQVIDEAVLDDIIRRLTEVRLARPGKQVQLSESEIKQLCVASRDIFINQPNLLELEAPIKICGDIHGQYSDLLRLFEYGGLPPTANYLFLGDYVDRGKQSLETICLLLAYKIKYPENFFLLRGNHECASINRIYGFYDECKRRFNVRLWKAFTDCFNCLPVAALIDEKILCMHGGLSPELTNLDEIRNLPRPTAIPDTGLLCDLLWSDPGRDVKGWGMNDRGVSYTFGPDMVAEFLTKHDLDLVCRAHQVVEDGYEFFADRKLVTIFSAPNYCGEFDNAGAMMSVDENLMCSFQILKPAEKKSKFVMSNKM

SEQ ID NO: 9 - protein, 326 aa, Glycine max
MDQALVDDIINRLLEVRGRPGKQVQLSESEIRQLCAASREIFLQQPNLLELEAPIKICGDVHGQYSDLLRLFEYGGLPPEANYLFLGDYVDRGKQSLETICLLLAYKIKYPENFFLLRGNHECASINRIYGFYDECKRRFNVRLWKTFTECFNCLPVAALIDEKILCMHGGLSPDLLNLDQIRNLQRPTDVPDTGLLCDLLWSDPSKEVQGWGMNDRGVSYTFGADKVSEFLQKHDLDLICRAHQVVEDGYEFFANRQLVTIFSAPNYCGEFDNAGAMMSVDETLMCSFQILKPADKKVKLNFGSTTTTKPGNSPAGVKSFLGTKV

SEQ ID NO: 10 - protein, 326 aa, Glycine max
MDQALLDDIINRLLEVRSRPGKQVQLSESEIRHLCAASREIFLQQPNLLELEAPIKICGDVHGQYSDLLRLFEYGGLPPEANYLFLGDYVDRGKQSLETICLLLAYKIKYPENFFLLRGNHECASINRIYGFYDECKRRFNVRLWKTFTECFNCLPVAALIDEKILCMHGGLSPDILNLDQIRNLQRPTDVPDTGLLCDLLWSDPSKEVQGWGMNDRGVSYTFGADKVSEFLQKHDLDLICRAHQVVEDGYEFFANRQLVTIFSAPNYCGEFDNAGAMMSVDETLMCSFQILKPADKKAKLNFGSTTTTKPGNSPAGIKSFLGTKV

SEQ ID NO: 11 - protein, 326 aa, Glycine max
MEQSVLDDIINRLLEVRTRPGKQVQLSESEIRQLCVVSREIFLQQPNLLELEAPIKICGDVHGQYSDLLRLFEYGGLPPEANYLFLGDYVDRGKQSLETICLLLAYKIKYPENFFLLRGNHECASINRIYGFYDECKRRFNVRLWKTFTDCFNCLPVAARVDEKILCMHGGLSPDLNNLDQIRNLQRPTDVPDTGLLCDLLWSDPSRDVQGWGMNDRGVSFTFGADKVSEFLQKHDLDLICRAHQVVEDGYEFFANRQLVTIFSAPNYCGEFDNAGAMMSVDETLMCSFQILKPADKKAKLNFGSTTTAKPGNSPAGVKCFLGAKV

SEQ ID NO: 12 - protein, 323 aa, Glycine max
MEQSLLDDIINRLLEVPTLPAKQVQLSESEIRQLCVVSREIFLQQPNLLELEAPIKICGDVHGQYSDLLRLFEYGGLPPEANYLFLGDYVDRGKQSLETICLLLAYKIKYPENFFLLRGNHECASINRIYGFYDECKRRFNVRLWKTFTDCFNCLPVAALVDEKILCMHGGLSPDLNNLDQIRNLQRPTDVPDTGLLCDLLWSDPSKDVQGWGMNDRGVSYTFGADKVSQFLQKHDLDLVCRAHQVVEDGYEFFANRQLVTIFSAPNYCGEFDNAGAMMSVDETLMCSFQILKPADKKAKLNFGSTTTAKPGNSPAGVKVGRY

SEQ ID NO: 13 - protein, 316 aa, Oryza sativa
MDPVLLDDIIRRLIEVKNLKPGKNAQLSESEIKQLCATSKEIFLNQPNLLELEAPIKICGDVHGQYSDLLRLFDYGGYPPQSNYLFLGDYVDRGKQSLETICLLLAYKIKYPENFFLLRGNHECASVNRIYGFYDECKRRFSVKLWKTFTDCFNCLPVAALIDEKILCMHGGLSPELNKLDQILNLNRPTDVPDTGLLCDLLWSDPSNDAQGWAMNDRGVSYTFGPDKVSEFLEKHDLDLICRAHQVVEDGYEFFANRQLVTIFSAPNYCGEFDNAGAMMSVDDTLMCSFQILKPARKMLGGSTNSKSGFKSLRGW

SEQ ID NO: 14 - protein, 367 aa, Sorghum bicolor
MDPALLDDVIRRLLEVKNLKPGKNAQLSESEIKQLCAAAKEIFLSQPNLLELEAPIKICGDVHGQYSDLLRLFDYGGYPPHANYLFLGDYVDRGKQSLETICLLLAYKVKYPENFFLLRGNHECASVNRIYGFYDECKRRFSVKLWKTFTDCFNCLPVSALIDEKILCMHGGLSPELNKLEQILNLNRPTDVPDTGLLCDLLWSDPSNEATGWAMNDRGVSFTFGPDKVNEFLEKHDLDLICRAHQVVEDGYEFFANRQLVTIFSAPNYCGEFDNAGAMMSVDETLMCSFQILKPARKMLGGSTNNKSGFKVCMFNMEAVYICQISTICCPTIFLVPLDISVLNFSRHYNMIVWHPSVTVCFKMMNE

SEQ ID NO: 15 - protein, 317 aa, Zea mays
MMDPALLDDVIRRLLEVKNLKPGKNAQLSESEIKQLCAAAKEIFLHQPNLLELEAPIKICGDVHGQYSDLLRLFDYGGYPPQANYLFLGDYVDRGKQSLETICLLLAYKVKYPENFFLLRGNHECASVNRIYGFYDECKRRFSVKLWKTFTDCFNCLPVSALIDEKILCMHGGLSPELNKLEQILNLSRPTDVPDTGLLCDLLWSDPSNEATGWAINDRGVSYTFGPDKVSEFLEKHDLDLICRAHQVVEDGYEFFASRQLVTMFSAPNYCGEFDNAGAMMSVDETLMCSFQILKPARKVLGGSTNNKSGFKSSRGW

SEQ ID NO: 16 - protein, 343 aa, Zea mays
MDPALLDDVIRRLLEVKNLKPGKNAQLSESEIKQLCAAAKEIFLQQPNLLELEAPIKICGDVHGQYSDLLRLFDYGGYPPQANYLFLGDYVDRGKQSLETICLLLAYKVKYPENFFLLRGNHECASVNRIYGFYDECKRRFSVKLWKTFTDCFNCLPVSALIDEKILCMHGGLSPELNKLEQILNLNRPTDVPDTGLLCDLLWSDPSNEATGWAINDRGVSFTFGPDKVSEFLEKHDLDLICRAHQVVEDGYEFFASRQLVTIFSAPNYCGEFDNAGAMMSVDDTLMCSFQILKPARKMMGGSTNNKSGFKVCMINMEAVYISRIKHDMLPYKIVWSLPSGYK

SEQ ID NO: 17 - protein, 318 aa, Arabidopsis thaliana
MAEKPAQEQEQKRAMEPAVLDDIIRRLVEFRNTRPGSGKQVHLSEGEIRQLCAVSKEIFLQQPNLLELEAPIKICGDIHGQYSDLLRLFEYGGFPPEANYLFLGDYVDRGKQSLETICLLLAYKIKYPENFFLLRGNHESASINRIYGFYDECKRRFNVRLWKIFTDCFNCLPVAALIDDRILCMHGGISPELKSLDQIRNIARPMDIPESGLVCDLLWSDPSGDVGWGMNDRGVSYTFGADKVAEFLEKHDMDLICRAHQVVEDGYEFFAERQLVTVFSAPNYCGEFDNAGAMMSIDESLMCSFQILKPSEKKSPFL

SEQ ID NO: 18 - protein, 312 aa, Arabidopsis thaliana
MAQQGQGSMDPAVLDDIIRRLLDYRNPKAGTKQAMLNDSEIRQLCFVSREIFLQQPCLLELAAPVKICGDIHGQYSDLLRLFEYGGFPPAANYLFLGDYVDRGKQSLETICLLLAYKIKYPENFFLLRGNHECASINRIYGFYDECKRRFNVKLWKVFTDTFNCLPVAAVIDEKILCMHGGLSPELINVEQIKNIERPTDVPDAGLLCDLLWSDPSKDVKGWGMNDRGVSYTFGADKVAEFLIKNDMDLVCRAHQVVEDGYEFFADRQLVTMFSAPNYCGEFDNAGALMSVDESLMCSFQILKPVDRRSRFF

SEQ ID NO: 19 - protein, 312 aa, Arabidopsis thaliana
MAQQGQGSMDPAALDDIIRRLLDYRNPKPGTKQAMLNESEIRQLCIVSREIFLQQPNLLELEAPIKICGDIHGQYSDLLRLFEYGGFPPTANYLFLGDYVDRGKQSLETICLLLAYKIKYPENFFLLRGNHECASINRIYGFYDECKRRFSVRLWKVFTDSFNCLPVAAVIDDKILCMHGGLSPDLTNVEQIKNIKRPTDVPDSGLLCDLLWSDPSKDVKGWGMNDRGVSYTFGPDKVAEFLIKNDMDLICRAHQVVEDGYEFFADRQLVTIFSAPNYCGEFDNAGAMMSVDESLMCSFQILKPADRKPRFL

SEQ ID NO: 20 - protein, 331 aa, Arabidopsis thaliana
MDPGTLNSVINRLLEAREKPGKIVQLSETEIKQLCFVSRDIFLRQPNLLELEAPVKICGDIHGQYPDLLRLFEHGGYPPNSNYLFLGDYVDRGKQSLETICLLLAYKIKFPENFFLLRGNHESASINRIYGFYDECKRRFSVKIWRIFTDCFNCLPVAALIDERIFCMHGGLSPELLSLRQIRDIRRPTDIPDRGLLCDLLWSDPDKDVRGWGPNDRGVSYTFGSDIVSGFLKRLDLDLICRAHQVVEDGFEFFANKQLVTIFSAPNYCGEFDNAGAMMSVSEDLTCSFQILKSNDKKSKFSFGSRGGAKTSFPYPKVKSILSSQNSKEYN

SEQ ID NO: 21 - protein, 330 aa, Glycine max
MERGVLDSIINRLLEVRGRPGKQVQLSEAEIKQLCLVSRDIFLRQPNLLELEAPIKICGDVHGQYSDLLRLFEYGGLPPRSNYLFLGDYVDRGKQSLETICLLLAYKIKYPNNFFLLRGNHECASINRIYGFYDECKRRYNVRLWKVFTECFNCLPVAALIDEKILCMHGGLSPELHNLNQIKSLPRPIEVPETGLLCDLLWSDPSSDIRGWGENDRGVSYTFGADRVTEFLQKHDLDLICRAHQVMEDGYEFFANRQLVTIFSAPNYCGEFDNAGAMMTVDETLVCSFQILKPVENKKPNKFAFGSTTTVKHSTPTKTKFQQSFFGAKA

SEQ ID NO: 22 - protein, 329 aa, Glycine max
MERGVIDNIINRLLQVRGRPGKQVQLSEAEIKQLCLVSRDIFMRQPNLLELEAPIKICGDIHGQYSDLLRLFEYGGLPPRYNYLFLGDYVDRGKQSLETICLLLSYKIKYPNNFFLLRGNHECASINRIYGFYDECKRRYNVRLWKVFTECFNCLPVAALIDEKILCMHGGLSPELHNLNQIKGLPRPIEVPETGLLCDLLWSDPSSDIRGWGENERGVSYTFGADRVTEFLQKHDLDLICRAHQVVEDGYEFFANRQLVTIFSAPNYCGEFDNAGAMMTVDETLVCSFQILKPVENKKPSKFGFGSTTTVKQSTTKAKFQQSFFGAKA

SEQ ID NO: 23 - protein, 301 aa, Glycine max
MERGVLDGIINRLLQVRGRPGKQVQLSEAEIRQLCAVSRDIFLKQPNLLELEAPIKICGDIHGQYSDLLRLFEHGGFPPRSNYLFLGDYVDRGKQSLETMCLLLAYKIKYPENFFLLRGNHECASVNRVYGFYDECKRRFNVRLWKIFADCFNCMPVAAIIEEKIFCMHGGLSPELHNLSQISSLPRPTEVPESGLLCDLLWSDPSKDIEGWGENDRGVSYTFGASRVTEFLGKHDLDLICRAHQVVEDGYEFFANRQLVTIFSAPNYCGEFDNAGAMMSVDETLMCSFQILRPAEHRKPK

SEQ ID NO: 24 - protein, 319 aa, Glycine max
MERGVLDGIISRLLQVRVRPGKQVQLSEAEIRQLCAVSRDIFLKQPNLLELEPPIKICGDIHGQYSDLLRLFEHGGLPPRSNYLFLGDYVDRGKQSLETICLLLAYKIKYPENFFLLRGNHECASINRVYGFYDECKRRFNVRLWKIFADCFNCMPVAAIIEEKIFCMHGGLSPELHNLSQISSLPRPTEVPESGLLCDLLWSDPSKDIEGWGDNERGVSYTFGASRVTEFLGKHDLDLICRAHQVVEDGYEFFSNRQLVTIFSAPNYCGEFDNAGAMMTVDETLMCSFQILRPVEHRKPKFGFGSKTTFKAVLDAARV

SEQ ID NO: 25 - protein, 321 aa, Glycine max
MDENLLDDIIRRLVAAKNGRTTKQVQLTEAEIRQLCVSSKEIFLSQPNLLELEAPIKICGDVHGQYSDLLRLFEYGGYPPEANYLFLGDYVDRGKQSIETICLLLAYKIKYKENFFLLRGNHECASINRIYGFYDECKRRFNVRLWKTFTDCFNCLPVAALIDEKILCMHGGLSPDLKHLDQIRSIARPIDVPDHGLLCDLLWADPDKDLDGWGENDRGVSFTFGADTVVEFLEHHDLDLICRAHQVVEDGYEFFAKRQLVTIFSAPNYCGEFDNAGAMMSVDDTLTCSFQILKSSEKKGKGGFGINTSRPGTPPHKGGKN

SEQ ID NO: 26 - protein, 321 aa, Glycine max
MDENVLDDIIRRLLAAKNGRTTKQVLLTEAEIRQLCVSSKEIFLSQPNLLELEAPIKICGDVHGQYSDLLRLFEYGGYPPEANYLFLGDYVDRGKQSIETICLLLAYKIKYKENFFLLRGNHECASINRIYGFYDECKRRFNIRLWKTFTDCFNCLPVAALVDEKILCMHGGLSPDLKHLDQIRSIARPIDVPDHGLLCDLLWADPDKDLDGWGENDRGVSFTFGADKVAEFLEHHDLDLICRAHQVVEDGYEFFAKRQLVTIFSAPNYCGEFDNAGAMMSVDDTLTCSFQILKSSEKKGKCGFGNNTSRPGTPPHKGGKN

SEQ ID NO: 27 - protein, 322 aa, Arabidopsis thaliana
MDETLLDDIIRRLLATNNGRTVKQAQITETEIRQLCLASKEVFLSQPNLLELEAPIKICGDVHGQFPDLLRLFEYGGYPPAANYLFLGDYVDRGKQSIETICLLLAYKVKYKFNFFLLRGNHECASINRVYGFYDECKRRYNVRLWKTFTECFNCLPVSALIDDKILCMHGGLSPDIKSLDDIRRIPRPIDVPDQGILCDLLWADPDREIQGWGENDRGVSYTFGADKVAEFLQTHDLDLICRAHQVVEDGYEFFAKRQLVTIFSAPNYCGEFDNAGALMSVDDSLTCSFQILKASEKKGRFGFNNNVPRPGTPPHKGGKGR

SEQ ID NO: 28 - protein, 322 aa, Arabidopsis thaliana
MEDSVVDDVIKRLLGAKNGKTTKQVQLTEAEIKHLCSTAKQIFLTQPNLLELEAPIKICGDTHGQFSDLLRLFEYGGYPPAANYLFLGDYVDRGKQSVETICLLLAYKIKYKENFFLLRGNHECASINRIYGFYDECKKRYSVRVWKIFTDCFNCLPVAALIDEKILCMHGGLSPELKHLDEIRNIPRPADIPDHGLLCDLLWSDPDKDIEGWGENDRGVSYTFGADKVEEFLQTHDLDLICRAHQVVEDGYEFFANRQLVTIFSAPNYCGEFDNAGAMMSVDDSLTCSFQILKASEKKGNFGFGKNAGRRGTPPRKGGGKG

SEQ ID NO: 29 - protein, 304 aa, Zea mays
MDRGTVEDLIRRLLDGKKHKATGKKVQLTETEIRHLCVTAKEIFLSQPNLLELVAPINVCGDIHGQFSDLLRLFEYGGLPPTANYLFLGDYVDRGKQSIETICLLLAYKIRYPDNFFLLRGNHECASINRIYGFYDECKRRFSVRLWKIFTDCFNCLPVAAVIDDKILCMHGGLSPDLDNLNRIREIQRPVDVPDQGLLCDLLWSDPDRDSSGWGDNDRGVSFTFGADKVTEFLNKHDLDLVCRAHQVVEDGYEFFADRQLVTIFSAPNYCGEFNNAGALMNVDASLLCSFQILKPYRGKAQTE

SEQ ID NO: 30 - protein, 305 aa, Sorghum bicolor
MDGGTVEDLIRRLLDGKKHKVTGKKAVQLTEPEIRHLCVTAKEVFLSQPNLLELEAPINVCGDIHGQFSDLLRLFEYGGLPPTANYLFLGDYVDRGKQSIETICLLLAYKIRYPDNFFLLRGNHECASINRIYGFYDECKRRFSVRLWKLFTDCFNCLPVAAVIDDKILCMHGGLSPDLDNLNRIREIQRPVDVPDQGLLCDLLWSDPDRDSSGWGDNDRGVSFTFGADKVTEFLNKQDLDLVCRAHQVVEDGYEFFADRQLVTIFSAPNYCGEFNNAGALMNVDASLLCSFQILKPYRGKAQTE

SEQ ID NO: 31 - protein, 307 aa, Oryza sativa
MMDGNAVDELIRRLLDGKKVKPSSSAKKVQLSEAEIRQLCVTGKDIFLSQPNLLELEAPINVCGDIHGQFSDLLRLFEFGGLPPTANYLFLGDYVDRGKQSIETICLLLAYKIKYPDNFFLLRGNHECASINRIYGFYDECKRRFSVRLWKLFTDCFNCLPVAAVIDDKILCMHGGLSPDLDSLDRIREIARPVDVPDQGLLCDLLWSDPDRESSGWGENDRGVSFTFGADKVTEFLNKHDLDLICRAHQVVEDGYEFFADRQLVTIFSAPNYCGEFNNAGALMNVDASLLCSFQILKPFRGKSQAE

SEQ ID NO: 32 - protein, 302 aa, Glycine max
MEGLDGLIERLLEVRKNRGKQIQLVESEIRSLCSTAKDLFLRQPNLLELEAPINVCGDIHGQYPDLLRVLEYGGFPPDSNYLFLGDYVDRGKQSVETICLLLAYKIKYPENFFLLRGNHECASINRIYGFYDECKRRFSVRLWKIFTDCFNCLPVAAVIDDKILCMHGGLSPDMESLNQIKAIERPVDVPDQGLLCDLLWADPDNEISGWGENDRGVSYTFGPDKVSEFLKKHDLDLICRAHQVVEDGYQFFADRQLVTIFSAPNYCGEFNNAGALMCVDQTLLCSFQIVKPFGTFRGKLTS

SEQ ID NO: 33 - protein, 322 aa, Oryza sativa
MDAAALDDLIRRLLDARGGRTARPAQLADAEIRKLCAAAKDVFLSQPNLLELEAPIKICGDIHGQYSDLLRLFEYGGFPPEANYLFLGDYVDRGKQSIETICLLLAYKIKYPENFFLLRGNHECASINRIYGFFDECKRRFNVRIWKVFTDCFNCLPVAALIDDKILCMHGGLSPDLKNMDQIRNIARPVDVPDHGLLCDLLWSDPDKEIEGWGENDRGVSYTFGADKVAEFLQTHDLDLICRAHQVVEDGYEFFAKRQLVTIFSAPNYCGEFDNAGAMMSIDDSLTCSFQILKPSDKKGKAGTGNMSKPGTPPRKIKINII

SEQ ID NO: 34 - protein, 324 aa, Zea mays
MDEAAVDDLIRRLLEARGGRTPRNAQVTDAEIRRLCAAAKDVFLSQPNLLELEAPIKICGDVHGQYSDLLRLFEYGGYPPDANYLFLGDYVDRGKQSIETICLLLAYKIKYPENFFLLRGNHECASINRIYGFFDECKRRFNVRIWKIFTECFNCLPVAALIDDKIFCMHGGLSPELKSMDQIRNISRPVDVPDVGLLCDLLWSDPDKEIDRWGENDRGVSYTFGADVVAEFLQKHDLDLICRAHQVVEDGYEFFAKRQLVTIFSAPNYCGEFDNAGALMSIDNSLVCSFQILKPSEKKGKAGNGNMPKPGTPPRKIKISVTHI

SEQ ID NO: 35 - protein, 324 aa, Sorghum bicolor
MDEAAVDDLIRRLLEARGGRTPRNAQVTDADIRRLCAAAKDVFLQQPNLLELEAPIKICGDVHGQYSDLLRLFEYGGYPPDANYLFLGDYVDRGKQSIETICLLLAYKLKYPENFFLLRGNHECASINRIYGFFDECKRRFNVRIWKIFTECFNCLPVAALIDDKIFCMHGGLSPELKNMDQIRNISRPVDVPDVGLLCDLLWSDPEKELDGWGENDRGVSYTFGADIVAEFLQKHDLDLICRAHQVVEDGYEFFANRQLVTIFSAPNYCGEFDNAGALMSIDDSLVCSFQILKPSEKKGKAGTSNMSKPGTPPRKIKISVTRI

SEQ ID NO: 36 - protein, 325 aa, Zea mays
MMDPALLDDVIRRLLEVKNLKPGKNAQVTDAEIRRLCATAKDVFLSQPNLLELEAPIKICGDVHGQYSDLLRLFEYGGYPPDANYLFLGDYVDRGKQSIETICLLLAYKIKYPENFFLLRGNHECASINRIYGFFDECKRRFNVRIWKIFTECFNCLPVAALIDDKIFCMHGGLSPELKNMDQIRNISRPVDVPDVGLLCDLLWSDPDKEIVGWGENDRGVSYTFGADIVAEFLQKHDLDLICRAHQVVEDGYEFFANRRLVTIFSAPNYCGEFDNAGALMSIDDSLVCSFQILKPSEKKRKAGAENMPKPGTPPRKIKISVTRI

SEQ ID NO: 37 - protein, 324 aa, Zea mays
MDEAAIDDLIRRLLEARGGRTPRNAQVTDAEIRRLCATAKDVFLSQPNLLELEAPIKICGDVHGQYSDLLRLFEYGGYPPDANYLFLGDYVDRGKQSIETICLLLAYKIKYPENFFLLRGNHECASINRIYGFFDECKRRFNVRIWKIFTECFNCLPVAALIDDKIFCMHGGLSPELKNMDQIRNISRPVDVPDVGLLCDLLWSDPDKEIVGWGENDRGVSYTFGADIVAEFLQKHDLDLICRAHQVVEDGYEFFANRQLVTIFSAPNYCGEFDNAGALMSIDDSLVCSFQILKPSEKKRKAGAENMPKPGTPPRKIKISVTRI

SEQ ID NO: 38 - protein, 318 aa, Arabidopsis thaliana
MMTSMEGMMEMGVLDDIIRRLLEGKGGKQVQLSEVEIRQLCVNARQIFLSQPNLLELHAPIRICGDIHGQYQDLLRLFEYGGYPPSANYLFLGDYVDRGKQSLETICLLLAYKIRYPSKIFLLRGNHEDAKINRIYGFYDECKRRFNVRLWKIFTDCFNCLPVAALIDEKILCMHGGLSPELENLGQIREIQRPTEIPDNGLLCDLLWSDPDQKNEGWTDSDRGISCTFGADVVADFLDKNDLDLICRGHQVVEDGYEFFAKRRLVTIFSAPNYGGEFDNAGALLSVDQSLVCSFEILKPAPASSTNPLKKVPKMGKS

SEQ ID NO: 39 - protein, 318 aa, Arabidopsis thaliana
MMTSMEGMVEKGVLDDIIRRLLEGKGGKQVQLSESEIRQLCFNARQIFLSQPNLLDLHAPIRICGDIHGQYQDLLRLFEYGGYPPSANYLFLGDYVDRGKQSLETICLLLAYKIRYPSKIYLLRGNHEDAKINRIYGFYDECKRRFNVRLWKVFTDCFNCLPVAALIDEKILCMHGGLSPDLDNLNQIREIQRPIEIPDSGLLCDLLWSDPDQKIEGWADSDRGISCTFGADKVAEFLDKNDLDLICRGHQVVEDGYEFFAKRRLVTIFSAPNYGGEFDNAGALLSVDESLVCSFEIMKPAPASSSHPLKKVPKMGKS

SEQ ID NO: 40 - protein, 375 aa, Glycine max
MLRLGHLVHLSKFPHPATNLFIYFSFIYLLSLARASKRVVFFFYKKKRRGKMMMMTMEGMMDKGVLDDVIRRLLEGKGGKQVQLSESEIRQLCVNARQIFLSQPILLDLRAPIRICGDIHGQYQDLLRLFEYGGYPPAANYLFLGDYVDRGKQSLETICLLLAYKIRYPDKIYLLRGNHEEAKINRIYGFYDECKRRFNVRLWKIFTDCFNCLPVAALIDEKILCMHGGLSPELENLDQIREIQRPTEIPDSGLLCDLLWSDPDASIEGWAESDRGVSCTFGADVVAEFLDKNDVDLVCRGHQVVEDGYEFFAKRRLVTIFSAPNYGGEFDNAGALLSVDDSLVCSFEILKPADRASGSGSSKMNFKKPPKLGKI

SEQ ID NO: 41 - protein, 324 aa, Glycine max
MMMMTMEGMMDKGVLDDVIRRLLEGKGGKQVQLSESEIRQLCVNARQIFLSQPILLDLRAPIRVCGDIHGQYQDLLRLFEYGGYPPAANYLFLGDYVDRGKQSLETICLLLAYKIRYPDKIYLLRGNHEEAKINRIYGFYDECKRRFNVRLWKIFTDCFNCLPVAALIDEKILCMHGGLSPELQNLDQIREIQRPTEIPDNGLLCDLLWSDPDASIEGWAESDRGVSCTFGADVVAEFLDKNDLDLVCRGHQVVEDGYEFFAKRRLVTIFSAPNYGGEFDNAGALLSVDDSLVCSFEILKPADRASGSGSSKMNFKKPPKLGKI

SEQ ID NO: 42 - protein, 325 aa, Oryza sativa
MMMTRASMGAMEGAAVDEVVRRLVEGGRGGRQVQLSEAEIRQLCVEAKRVLLSQPNLLRIHAPVKICGDIHGQFVDLLRLFDLGGYPPTSTYLFLGDYVDRGKQSLETICLLLAYKVKYPDKIFLLRGNHEDAKINRVYGFYDECKRRFNVRLWKIFCDCFNCLPMAALIDDKILCMHGGLSPELNSLDQIKDIERPTEIPDYGLLCDLVWSDPSPDSEGWGESDRGVSCTFGADKLVEFLEKNDLDLICRAHQVVEDGYEFFAQRRLVTIFSAPNYCGEFDNAGALLSIDESLMCSFQILKPNDTGAPHSRKPTSNKTPKTGNA

SEQ ID NO: 43 - protein, 325 aa, Sorghum bicolor
MMMTRASMGAMDAAAVDEVVRRLVEGGRGGRQVQLSEAEIRQLCVEAKQVLLSQPNLLRIHAPVKICGDIHGQFVDLLRLFDLGGYPPTSTYIFLGDYVDRGKQSLETICLLLAYKLKYPDNIYLLRGNHEDAKINRVYGFYDECKRRFNVRLWKIFCDCFNCLPMSALIDDKVLCMHGGLSPELNSLDQIKDIERPTEIPDYGLLCDLLWSDPSHDTEGWGESDRGVSCTFGADKLVEFLDKNDLDLVCRAHQVVEDGYEFFAERRLVTIFSAPNYCGEFDNAGALLSIDESLMCSFQILKPNETGGPHSRKPLPSKAPKGENV

SEQ ID NO: 44 - protein, 322 aa, Zea mays
MMMTRAPMGPMEGAAVDEMVRRLVEGGRGGRQVQLSEAEIRQLCVEGKRVLLSQPNLLRIHAPVKICGDIHGQFVDLLRLFDLGGYPPASTYVFLGDYVDRGKQSLETICLLLAYKIRYPENIFLLRGNHEDAKINRVYGFYDECKRRFNVRLWKIFSDCFNCLPIAALIDDKILCMHGGLSPELTSLDQIKDIERPAEIPDYGLLCDLLWSDPSPDGEGWGESDRGVSCTFGADKLVEFLEKNDLDLICRAHQVVEDGYEFFAQRRLVTIFSAPNYCGEFDNVGALLSIDESLMCSFQILKPTDMGPPHARKQIPNKPTRG

SEQ ID NO: 45 - protein, 351 aa, Zea mays
MMMTRAPMGPMEGAAVDEMVRRLVEGGRGGRQVQLSEAEIRQLCVEGKRVLLSQPNLLRIHAPVKICGDIHGQFVDLLRLFDLGGYPPASTYVFLGDYVDRGKQSLETICLLLAYKIRYPENIFLLRGNHEDAKINRVYGFYDECKRRFNVRLWKIFSDCFNCLPIAALIDDKILCMHGGLSPELTSLDQIKDIERPAEIPDYGLLCDLLWSDPSPDGEGWGESDRGVSCTFGADKLVEFLEKNDLDLICRAHQVVEDGYEFFAQRRLVTIFSAPNYCGEFDNVGALLSIDESLMCSFQILKPTDMGPPHARKQIPNKVNQLPSHRNIGLIFSYKATTLEITCLFYYYHVS

SEQ ID NO: 46 - protein, 322 aa, Zea mays
MMMTRAPMGPMEGAAVDEVVRRLVEGGRGGRQVQLSEAEIRQLCVDGKRVLLSQPNLLRIHAPVKICGDIHGQFVDLLRLFDLGGYPPASTYVFLGDYVDRGKQSLETICLLLAYKIRYPEKIFLLRGNHEDAKINRVYGFYDECKRRFNVRLWKIFSDCFNCLPIAALIDDKILCMHGGLSPEWTSLDQIKDIERPAEIPDYGLLCDLLWSDPSPDGEGWGESDRGVSCTFGADKLVEFLEKNDLDLICRAHQVVEDGYEFFAQRRLVTIFSAPNYCGEFDNAGALLSIDESLMCSFQILKPTDMGPPHARKQIPNKPARG

SEQ ID NO: 47 - protein, 322 aa, Sorghum bicolor
MMMTRAPMGPMEGAAVDEVVRRLVEGGRGGRQVQLSEAEIRQLCVEGKRVLLSQPNLLRIHAPVKICGDIHGQFVDLLRLFDLGGYPPASTYVFLGDYVDRGKQSLETICLLLAYKIRYPEKIFLLRGNHEDAKINRVYGFYDECKRRFNVRLWKIFSDCFNCLPIAALIDDKILCMHGGLSPELTSLDQIKDIERPAEIPDYGLLCDLLWSDPSPDGEGWGESDRGVSCTFGADKLVEFLEKNDLDLICRAHQVVEDGYEFFAQRRLVTIFSAPNYCGEFDNAGALLSIDESLMCSFQILKPTDMGPPHARKQIPNKPARG

SEQ ID NO: 48 - DNA, 972 bp, Zea mays
atgtcggcggcgccagcggcgggagggcagggaggcgggggtatggacgccgcgctcctcgacgacatcatccgccgcctgctcgaggtgcggaccgcgcgccccgggaagcaggtgcagctctccgagtcggagatccgccagctctgcaccgtctcccgcgagatcttcctcaaccaacccaaccttctcgagctcgaggcgcccatcaagatctgcggtgacatccatggtcaatacagtgatcttctaaggctttttgaatatggaggctttcctcctgaagccaattatctattcttaggtgattatgttgatcgaggcaaacaaagtttggagactatatgccttcttcttgcttacaaaatcaagtaccctgagaacttttttcttctgaggggcaatcatgaatgcgcctcaataaacagaatatatggattctatgatgaatgcaagcgtcgatttaatgtgcggctatggaaggtcttcacagaatgttttaacactctacctgtggctgctcttatcgatgataaaatattatgtatgcacggtggactttctcccgatctagcacacttggatgagataaagaacttgcaacgtccaaccgatgtaccagatcaaggtctactgtgtgacttactttggtcagatccaggaaaagacgttcaggggtggggcatgaatgatagaggggtctcatatacctttggtgctgacaaggtttcagaattcctgcaaaggcatgatcttgatcttatttgtcgtgctcatcaggttgtcgaggatgggtatgaattctttgctgacagacagcttgtcaccatattctcggctcccaattattgtggtgaatttgataatgctggcgcaatgatgagcgttgacgaaactttgatgtgttccttccaaattctgaaacctgctgagagaaaacaaatttatggggccaaacaaaatgtgagatag

SEQ ID NO: 49 - DNA, 969 bp, Zea mays
atggcggcggcgccagcggcgggagggcagggaggcgggggtatggacgccgcgctcctcgacgacatcatacgccgcctgctcgaggtgcggaccgcgcgccccgggaagcaggtgcagctctccgagtcggagatccgccagctctgcaccgtctcccgcgagatcttcctcaaccaacccaaccttctcgagctcgaggcgcccatcaagatctgcggtgacatccatggtcaatacagtgatcttctaaggctttttgaatatggaggctttcctcctgaagccaattatctattcttaggtgattatgttgatcgaggcaaacaaagtttggagactatatgccttcttcttgcttacaaaatcaagtaccctgagaacttttttcttctgaggggcaatcatgaatgcgcctcaataaacagaatatatggattctatgatgaatgcaagcgtcgatttaatgtgcggctatggaaggtcttcacagaatgttttaacactctacctgtggctgctcttatcgatgataaaatattatgtatgcacggtggactctctcctgatctagcacacttggatgagataaagaacttgcagcgtccaactgatgtaccagatcaaggtctactgtgtgacttgctttggtcagatccaggaaaagatgctcaagggtggggcatgaatgatagaggggtctcatatacctttggtgctgacaaggtttcagaattcttgcaaaagcatgatcttgatcttatctgtcgtgctcaccaggttgtcgaggatgggtatgaattttttgctgacagacagcttgtcaccatattctcggcccccaattattgtggtgaatttgataatgctggtgcaatgatgagtgtcgatgaaactttgatgtgctcattccaaattctgaaacctgctgagagaaaaaacaaatttatggggtccaacaaaatgtga

SEQ ID NO: 50 - DNA, 972 bp, Zea mays
atgtcggcggcgccagcggcgggagggcagggtggcgggggcattgacgccgcgctcctcgacgacatcatccgccgcctgctcgaggtgcggaccgcgcgcccagggaagcaggtgcagctctctgagtcggagatccgccagctgtgcaccgtctcccgcgccatcttcctcagccagcccaacctcctcgagctcgaggcgcccatcaagatctgcggtgacatccatggtcaatacagtgatcttctaaggctttttgaatatggaggttttcctcctgaagccaattatctattcttaggtgattatgttgatcgaggcaaacaaagtttggagactatatgccttcttcttgcttacaaaatcaagtaccctgagaacttttttcttctgaggggcaatcatgaatgtgcctcaataaacagaatatatggattttatgatgaatgcaagcgtcgatttaatgtgcggctatggaaggtcttcacagaatgttttaacactctacctgtggctgctcttatcgatgataaaatattatgtatgcacggtggactctctcccgatctagcacacttggatgagataaagaacttgcaacgtccaaccgatgtaccagatcaaggtctactgtgtgacttactttggtcagatccaggaaaagatgttcaagggtggggcatgaatgatagaggggtctcatatacctttggtgctgacaaggtttcagaattcctgcaaaggcatgatcttgatcttatttgtcgtgctcatcaggttgtcgaggatgggtatgaattctttgctgacagacagcttgtcaccatattctcggctcccaattattgtggtgaatttgataatgctggcgcaatgatgagcgttgacgaaactttgatgtgttccttccaaattctgaaacctgctgagagaaaacaaatttatggggccaaacaaaatgtgagatag

SEQ ID NO: 51 - DNA, 969 bp, Sorghum bicolor
atggcggcggcgccagcggcgggagggcagggcggcgtgggcatggacaccgcgctcgtcgacgacatcatccgccgcctgctcgaggtgcggaccgcgcgccccgggaagcaggtgcagctctccgagtcggagatccgccagctctgcaacgtctcccgcgaaatcttcctcagccagcccaacctcctcgagctcgaggcgcccatcaagatctgcggtgacatccatggtcaatacagtgatcttctaaggctttttgaatatggaggctttcctcctgaagccaactatctattcttaggcgattatgttgatcgaggcaaacaaagtttggagactatatgccttcttcttgcttacaaaatcaagtaccctgagaacttttttcttctgaggggcaatcatgaatgtgcctcgataaacagaatatatggattttatgatgaatgcaagcgtcgatttaatgtgcggctatggaaggtcttcacagaatgttttaacactctacctgtggctgctcttatcgatgataaaatattatgtatgcacggcggactctcccctgatctagcacacttggatgagataaagagcttgcaacgtccaaccgatgtaccagatcaaggactactgtgtgatttgctttggtcagatccaggaaaagatgttcaagggtggggcatgaatgatagaggagtctcatatacctttggcgctgacaaggtttcagaattcttgcaaaagcatgatcttgatcttatttgtcgtgctcaccaggttgttgaagatgggtatgaattctttgctgacagacagcttgtcaccatattctcggcccccaattattgtggtgaatttgataatgctggtgcaatgatgagtgttgatgaaactttgatgtgttcgttccaaattctgaagcctgctgagagaaaaaacaaatttatggggtcaaacaaaatgtga

SEQ ID NO: 52 - DNA, 969 bp, Oryza sativa
atggcggcggcaccgggggcgggagggcagggcggcggcgggatggacgccgtcctcctcgacgatatcatccgccgcctgctcgaggtgcggacggcgcgcccggggaagcaggtgcagctctccgagtcggagatccgccagctctgcactgtatcccgagaaatcttcctcagccagcccaatctcctcgagctcgaggcgcccatcaagatctgcggtgacatccatggtcagtacagtgaccttctaaggctttttgagtatggtggttttcccccagaagccaattatctattcttaggtgattatgttgatcgaggcaaacaaagtttggaaacaatatgcctcctccttgcatacaaaatcaagtacccggagaatttttttcttctcagaggcaatcatgagtgtgcatcaataaacaggatatatggattttatgatgaatgcaaacgccgatttaatgtgcgtctatggaaggtcttcactgattgttttaactgtctccctgtggctgcgctaattgatgataaaatattatgcatgcatggtggcctctctcctgatctgacacatttagatgagataaagagcttgccccgtcctactgatgtaccggatacaggtctgctatgcgacctactttggtcagatccaggaaaagacgttcaaggttggggcatgaatgataggggggtctcatacacttttggtgctgacaaagtttcagaattcctggaaaaacatgaccttgatcttatttgccgggcacatcaggttgttgaggatggatatgagttctttgccgacagacaacttgtcaccatattctcagctccaaactattgtggtgaatttgataatgcaggtgcaatgatgagtgttgatgaaactttgatgtgttcttttcaaattcttaaacctgctgagagaaaaggcaaatttatggcatcaaacaaaatgtga

SEQ ID NO: 53 - DNA, 966 bp, Arabidopsis thaliana
atggcgacgacgacgacgacgcaggggcagcaaacggcgattgactcggcggttctcgatgatataatcaggcgactcacggaggtccggttagctagaccaggaaagcaagtccagctctctgaagctgagatcaaacagctctgtactacagctagagatattttccttcagcaacccaatttacttgagcttgaggctcccatcaaaatctgtggtgacatacatgggcaatatagtgatttgttgaggctatttgagtatggtggcttccctcctagtgccaattatctatttcttggggactatgtggacagaggcaaacagagtcttgaaacaatatgtctcttactagcttacaagatcaaataccctggcaacttttttctactaaggggaaaccacgaatgtgcttctatcaataggatatatggattttatgatgagtgtaaaaggaggttcaatgtgagagtatggaaggtttttactgattgttttaactgccttcctgttgcggcgcttatagatgacaaaatattgtgtatgcacggtggactttcgccggatcttgaccatttggatgagattagaaacttgccgagaccaactatgattccggataccgggcttctctgtgatttgctctggtctgatcctgggaaagatgttaaaggatggggaatgaatgatagaggtgtttcatacacctttggtccagataaagtttccgagtttcttacgaaacatgatttagatcttgtgtgtcgtgcccatcaggtcgtggaggatgggtatgagttctttgctgatagacaacttgtgacggtgttttcagctcctaactactgtggtgaatttgataatgctggtgcgatgatgagcgtggacgagaaccttatgtgctcgtttcagattttgaagcccgctgagaagaagaccaagttcatgatgtccacaaagatttga

SEQ ID NO: 54 - DNA, 951 bp, Glycine max
atgagcacacaggggcaagtgattattgatgaggcggttctggatgacataatccggcgcttaacggaggtccgactggcccgacccggcaagcaggttcagctctccgagtctgagatcaagcaactctgcgtcgcttccagagacatcttcattaaccagcccaatttgcttgaactcgaagcccccatcaagatttgtggtgacattcatgggcagtacagtgatttgttaaggctatttgagtatgggggtttgcctcctactgcgaattatctctttttgggggactacgtggaccgtgggaagcagagcttagaaaccatatgtcttttgcttgcgtataaaatcaaatatccagaaaactttttcctgttaagggggaatcatgagtgtgcttccattaataggatttatgggttttatgatgaatgtaagcgaaggtttaacgtgaggctttggaaagcctttaccgactgttttaacttccttcctgtggcagcccttatagatgataaaatattgtgcatgcatggtggtctttcccctgaactcacaaacttggatgaaatcaggaatctacctcgtcctactgcgattcccgacaccggcttgctttgtgatttgctttggtctgatcctggtagggatgtgaagggttggggtatgaatgacagaggagtgtcctacacctttggccctgataaggtcgctgagttcttgacaaagcatgacttggacctcatttgtcgtgctcatcaggttgtagaggatgggtatgaattctttgctgataggcaacttgttacgatattttcagctccaaactattgtggtgaatttgacaatgctggtgcgatgatgagtgtggacgaaaacttgatgtgctcatttcagattcttaagcctgcagagaaaaaatcaaagtttgtgatgtcaaacaagatgtga

SEQ ID NO: 55 - DNA, 948 bp, Glycine max
atgagcacacaaggacaagtgattgatgaggcggttctggatgacataatccggcgcctaacggaggtccgattggcccgacccggaaagcaggttcagctctccgagtctgagatcaagcaactttgcgtcgcttccagagacatcttcattaaccagcccaatttgcttgaactcgaagcccccatcaagatttgtggtgacattcatgggcagtacagtgatttgttaaggctatttgaatatgggggtttgcctcctactgccaattatctctttttgggggactatgtggaccgtgggaagcagagcttagaaaccatatgtcttttgcttgcgtataaaatcaaatatccagaaaactttttcctgttgagggggaatcatgagtgtgcttccattaataggatttatgggttttatgatgaatgtaagcgaaggtttaatgtgaggctttggaaagcctttaccgactgttttaactgccttcctgtggcagcccttatagatgaaaaaatattgtgcatgcatggtggtctttcccctgaactcacaaacttggatgaaatcaggaatctacctcgtcctactgcaattcccgacactggcttgctttgtgatttgctttggtctgatcctggtagggatgtgaagggttggggtatgaatgacagaggagtgtcctacacctttggccctgatatggtcgctgaattcttgacaaagcatgacttggaccttgtttgtcgtgctcaccaggttgtagaggatgggtatgaattctttgctgataggaaacttgttacgatattttcagctccaaactattgtggtgaatttgacaatgctggtgcgatgatgagtgtggatgaaaacttgatgtgctcatttcagattcttaagcctgcagagaaaaaatcaaagtttgtgatgtcaaacaagatgtga

SEQ ID NO: 56 - DNA, 981 bp, Glycine max
atggaccaagctcttgtggacgacataatcaaccgccttctcgaagttcgcggccgcccgggcaagcaggtgcaactgtcggagtcggagatccgccagctctgtgccgcttccagagagattttcctgcagcaacctaatttgttggagcttgaagcacccattaagatttgcggtgatgtacatggtcaatattctgatcttttaaggctttttgagtatggcggactacctcctgaggccaactacttatttttgggggattatgtggatcgagggaagcagagtttggaaacaatatgccttctcctagcatataaaataaaatatcccgagaattttttccttttgaggggtaaccatgaatgtgcttctattaaccgtatatatggattttatgatgagtgtaaacgaaggttcaatgtaaggttatggaagacatttacagaatgcttcaattgccttcctgtggcagcactgattgatgaaaagattttgtgcatgcatgggggtctttctcctgacttgcttaatttggatcagattagaaatctacaacggcccactgatgtgcctgatacaggtttgctttgtgatctcctctggtcagacccaagcaaagaggtccaaggttggggaatgaatgacaggggagtttcatatacatttggtgctgataaggtctcagagtttcttcaaaaacatgatttggatcttatttgtcgtgctcatcaggtggtggaagatgggtatgagttctttgctaaccggcagcttgtaacaatattttcagcacctaattattgtggagagtttgacaatgctggcgctatgatgagtgtcgatgagacactaatgtgctctttccaaatattaaagccagctgataaaaaagtaaagctcaacttcggaagtactactactactaagccaggaaactctccagcaggcgtaaagtccttcctgggtacaaaagtatga

SEQ ID NO: 57 - DNA, 981 bp, Glycine max
atggaccaagctcttctggacgacataatcaatcgccttctcgaagttcgcagccggccggggaagcaggtgcagctgtcggagtcggagatccgccatctctgtgccgcttccagagagattttcttgcagcaacctaatttgttggagcttgaagcacccattaagatttgcggtgatgtacatggtcaatattctgatcttttaaggctttttgagtatggtggactacctcccgaggccaactacttatttttgggggattatgtagatcgagggaagcagagtttggaaacaatatgccttctcctagcatataaaataaaatatcccgagaattttttccttttgaggggtaaccatgaatgtgcttctattaaccgtatatatgggttttatgacgagtgtaaaagaaggttcaatgtaaggttatggaagacatttacagaatgcttcaattgccttcctgtggcagcactaattgatgaaaagattttgtgcatgcatgggggtctttctcctgacatacttaatctggatcagattagaaatctacagcggcccactgatgtgcctgatacaggtttgctttgtgatctcctctggtcagacccaagcaaagaggtccaaggttggggaatgaatgacaggggagtttcatatacatttggtgctgataaggtctcagagtttcttcagaaacatgatttggatcttatttgtcgtgctcatcaggtggtggaggatgggtacgagttctttgctaaccggcagcttgtaacaatattttcagcacctaattattgtggagagtttgacaatgctggcgctatgatgagtgttgatgagacactaatgtgctctttccaaatattaaagccagctgataaaaaagcaaagctcaactttggaagtactactactactaagccaggaaactctccagcaggcataaagtccttcctgggtacaaaagtatga

SEQ ID NO: 58 - DNA, 981 bp, Glycine max
atggaacaatcggttttggatgatataatcaatcgcctcctcgaagttcgtacccgaccggggaagcaggttcagctatcggagtccgagatccgtcaactctgcgtagtttccagagagattttcttgcaacaacctaatttattagagcttgaagcacctattaagatttgtggtgatgtacatgggcaatattctgatcttttaaggctttttgagtacggtggattacctccagaagccaactacttgtttttgggggattatgtggatcgagggaagcaaagtttagaaacaatttgccttctccttgcttataaaataaaatatcctgagaactttttcctgttaaggggaaaccatgaatgtgcttctataaaccggatatatggattttatgatgagtgcaagagaaggttcaatgtaaggttatggaagacatttacagactgcttcaattgcctgcctgtggcagcccgtgttgatgaaaagattttgtgtatgcatggaggactttctcccgacttaaataatttggatcagattagaaatttacagcggcccacagatgttcctgatacaggtttgctttgtgatctcctttggtctgacccgagcagagatgttcaaggatggggaatgaatgacagaggagtttcatttacatttggtgctgataaggtctcagagtttcttcagaaacatgatcttgatcttatttgtcgtgctcatcaggttgtggaagatggatatgagttctttgctaatcgacaacttgtaacaatattttcagcacctaattattgtggggaatttgacaatgctggtgctatgatgagtgttgatgagacactaatgtgctctttccaaatattaaagccagctgataaaaaagcaaagctcaattttggaagtacaaccactgctaagcctggaaactccccagctggtgtaaagtgcttcctgggtgcgaaagtatga

SEQ ID NO: 59 - DNA, 972 bp, Glycine max
atggaacaatcgcttttggatgacataatcaatcgcctcctcgaagttcctaccctaccggctaagcaggttcagctatccgagtccgagatccgtcaactctgcgtagtttccagagaaattttcttgcaacaacctaatttattggagctcgaagcacctattaagatttgtggtgatgtacatgggcaatattctgatcttttaaggctttttgagtacggtggattacctcctgaagccaactatttgtttttgggggattatgttgatcgagggaagcagagtttagaaacaatttgcctcctccttgcttataaaataaaatatcctgagaactttttcttgttaaggggaaaccatgaatgtgcttctataaaccggatatatggattttatgatgagtgcaagagaaggttcaatgtaaggttatggaagacatttacagactgcttcaattgcctgcctgtggcagcccttgtcgatgaaaagattttgtgtatgcatgggggactttctcccgacttaaataatttggaccaaattagaaatttacagcggcccacagatgttcctgatacaggtttgctttgtgatctgctttggtctgacccgagcaaagatgttcaaggatggggaatgaatgacagaggagtttcatacacatttggtgctgataaggtctcacaatttcttcagaaacatgatcttgatcttgtttgtcgtgctcatcaggttgtggaagatggatacgagttctttgctaatcgacaacttgtaacaatattttcagcacctaattattgtggggagtttgacaatgctggtgctatgatgagtgttgatgagacgctaatgtgctctttccaaatattaaagccagctgataaaaaagcaaagctcaattttggaagtacaaccactgctaagcctggaaactctccagcaggtgtaaaggttggaagatattag

SEQ ID NO: 60 - DNA, 951 bp, Oryza sativa
atggatccggtgttgctggacgacatcatccggaggcttatcgaggtgaagaatctgaagccggggaagaacgcgcagctttcggagtcggagattaagcagctctgcgcaacctccaaggagatcttcctgaatcagcccaacctgctcgagctcgaggcccccatcaaaatctgcggtgatgttcatggacagtattctgatctcctgaggctgtttgattatggtgggtatccacctcagtccaactatctcttcttgggcgattatgtggaccggggaaagcaaagccttgagacgatatgccttcttttggcttataagatcaagtaccctgaaaacttcttcctactcagaggcaaccatgaatgtgcatcggtcaaccgcatctatggattttatgacgagtgcaagcgcagattcagtgtaaaactctggaagacttttactgactgttttaactgcttaccagtggcagcattgatagatgaaaagattctttgtatgcacggaggtctttctccagagttgaataagctggatcaaatactcaacctcaaccgccccacggatgtgcctgatactgggttactttgtgatctcctttggtccgatccatccaatgacgcacaagggtgggctatgaatgatcgaggtgtctcatatacattcgggccagacaaagtgtctgaatttcttgagaagcatgatttagacctcatctgtcgagcccatcaggttgtcgaagatgggtacgagttctttgctaaccgccaacttgtaacaatattctcggcccctaattactgtggagaatttgataatgctggtgccatgatgagtgtagatgatacactgatgtgctcttttcaaatactaaaaccagcgaggaaaatgttgggtggttccacgaattccaaatccggcttcaagtcactgagagggtggtga

SEQ ID NO: 61 - DNA, 1104 bp, Sorghum bicolor
atggatccggccttgcttgacgacgtcatacgccggcttctggaggtgaagaatctcaagcccggtaagaacgcgcagctgtcggagtcggagattaagcagctctgcgctgccgccaaggagatcttcctttcgcagcccaacctgctggagctcgaggcccccatcaaaatctgcggtgatgtccatgggcagtactctgatctcctgaggctatttgattatggtggctatccgcctcatgcgaactaccttttcttgggtgattatgtggatcgtggaaagcaaagcctggaaacaatatgtcttcttttggcctacaaggtcaagtacccggagaacttctttcttctaaggggcaaccatgaatgtgcatcagtaaatcgcatctatggtttttatgacgagtgcaaacgcagattcagtgtaaagctctggaaaacattcacagattgttttaactgcttgccagtgtcagcattgatagatgaaaagattctctgtatgcatggcggtctatctccagagttgaacaagcttgagcaaatactcaacctgaatcgccccacagatgtgcctgatactggattgctctgtgatcttctttggtctgatccttccaatgaagcaacaggctgggccatgaatgaccgaggtgtttcattcacatttggtcctgataaagttaatgaattccttgagaagcatgatttggacctcatctgccgagctcatcaggttgtcgaagatggatatgagttttttgctaaccgccaactcgtaacaatattctcagcccctaactactgtggagaattcgataatgctggtgccatgatgagtgtagatgagacattgatgtgctctttccaaatactgaagcctgcaaggaagatgctgggtggttcaactaataacaaatctggcttcaaggtatgcatgtttaacatggaagcagtttatatctgtcaaataagcacaatatgttgcccaacaatttttttggtccctctggatattagtgttcttaatttttcaaggcattataacatgatagtgtggcatcctagtgttactgtttgcttcaaaatgatgaatgaatga

SEQ ID NO: 62 - DNA, 954 bp, Zea mays
atgatggatccggccttgcttgacgacgtcatacgccggctgctggaggtgaagaatctcaagcctgggaagaacgcgcagttgtcggagtcggagatcaagcagctctgcgctgccgccaaggagatcttcctgcaccagcccaacctgctggagctcgaggcgcccatcaaaatctgcggtgatgtccatggccagtactctgatctcctgaggctattcgattatggtggctatccgcctcaggccaactaccttttcttgggtgattatgtggatcgtggaaagcaaagcttggaaacaatatgtcttcttttggcctacaaggtcaagtacccagagaacttctttcttctaaggggaaaccatgaatgcgcatcagtaaatcgcatctatggtttttatgacgagtgcaaacgcagattcagtgtaaagctgtggaaaacattcactgactgttttaactgcttgccagtgtcagcattgatagacgaaaagatcctctgtatgcatggtggtctatctccagagttgaacaagctcgaacaaatacttaatctgagtcgccccacagacgtgcctgatactggattgctctgtgaccttctttggtctgatccttctaatgaagcaacaggatgggccataaatgatcgaggtgtttcatatacatttggtcctgataaagtttctgaattccttgagaagcatgatttggacctcatctgccgagcccatcaggttgtcgaagatggatatgagtttttcgctagccgccaactcgtaacaatgttttcagcccctaactactgtggagaatttgataacgctggtgccatgatgagtgtagatgagacattgatgtgctccttccaaatactgaaacctgcgaggaaggtgttgggtggttcaactaataacaaatcaggcttcaagtcatcgagaggatggtga

SEQ ID NO: 63 - DNA, 1032 bp, Zea mays
atggatccggcgttgctggacgacgtcatacgccggcttctggaggtgaagaatctcaagcctgggaagaacgcgcagctgtcagagtcggagattaagcagctctgcgctgccgccaaggagatcttcctgcagcagcccaacctgctggagctcgaggcccccatcaaaatctgcggtgatgtccatggccagtactctgatctcctgaggctatttgattatggtggctatccgcctcaggccaactaccttttcttgggtgattatgtggatcgtggaaagcaaagcctagaaacaatatgtcttcttttggcttacaaggtcaagtacccggagaacttctttcttctaaggggcaaccatgaatgcgcatcagtaaatcgcatctatggtttttatgacgagtgcaaacgcagattcagtgtaaagctgtggaaaacattcacagactgttttaactgcttgccagtgtcagcattgatagatgaaaagattctctgtatgcatggtggtctatctccggagttgaacaagcttgagcaaatactcaacctgaatcgccccacagacgtgcctgatactggattgctctgtgatcttctttggtctgatccttccaatgaagcaacaggctgggccatcaatgatcgtggtgtttcattcacatttggtcctgataaagtttctgaatttcttgagaagcatgatttggaccttatctgccgagcacatcaggttgtcgaagatggatacgagtttttcgctagccgtcaactcgtaacaatattttcagcccctaactactgtggagaattcgacaacgctggtgccatgatgagtgtagatgatacattgatgtgctcgttccaaatattgaagcctgcaaggaagatgatgggtggttcaactaataacaaatctggcttcaaggtatgcatgattaacatggaagcagtttatatcagtcggataaaacacgatatgttgccctacaaaattgtttggtccctaccctctggatacaagtag

SEQ ID NO: 64 - DNA, 957 bp, Arabidopsis thaliana
atggcggagaagccggcgcaagagcaagagcagaagcgagcgatggaacctgcagttctcgacgatattattcgtcgtttggttgagtttcggaacacgagacctggatcggggaagcaagttcatctcagtgaaggtgaaattcgtcagctttgtgctgtctccaaagaaatatttcttcaacagcccaatctgcttgaattggaagctcccatcaagatctgcggtgatattcatgggcagtattcagatctattgaggctttttgagtatggagggttccctcccgaagctaattatttgttcttgggtgattatgttgaccgtggcaagcaaagcttggaaacaatatgtcttcttctagcttacaaaatcaagtaccctgagaacttcttcttgttgagagggaatcatgaatctgcttccattaatcgtatttacggtttctatgatgagtgcaaacgcaggttcaatgtcagactctggaaaatattcaccgattgctttaactgtcttcctgtggccgccttaattgatgacagaatactatgtatgcatggtgggatttccccagagctgaaaagtttggaccagattagaaatattgcacggccgatggatattccggagtctggtttggtatgtgatttactatggtcggatcctagtggagacgtaggctggggcatgaatgatcgtggtgtttcatacacttttggagctgacaaagtcgcagagttcttggagaaacatgacatggaccttatctgtcgtgcccaccaggttgttgaagatgggtatgagttctttgcagaaagacaacttgttacagtattttcagctcccaactattgcggggaatttgacaacgctggcgcaatgatgagcattgatgagagcttaatgtgctcattccagattctaaagccgtcagaaaagaagtcgccttttctgtga

SEQ ID NO: 65 - DNA, 939 bp, Arabidopsis thaliana
atggcgcaacaagggcagggaagcatggaccctgctgttctcgacgacatcattcgtcgtttacttgattaccgaaaccctaaagctgggaccaaacaagctatgctcaacgattctgagatccgacagctttgctttgtctctagagagatatttcttcaacagccttgtcttcttgagctcgctgcccctgttaagatctgcggtgatattcatggtcaatactcagatttattgaggcttttcgagtatggaggtttccctcctgcagcaaactacttattcttaggagattacgttgaccgcggaaagcagagtttggagactatttgtcttctgcttgcttacaagatcaaataccctgaaaacttttttctcttaagaggaaaccatgagtgtgcatctattaacagaatttacggattctatgacgaatgtaaacgaagattcaacgtgaagctctggaaagtgtttaccgatacttttaactgtctccctgtggctgctgtcatagatgaaaagatactttgtatgcatggtggactatctcctgagttgatcaatgtggaacaaattaagaacatagagcgtccaactgatgttccagacgctggtttgctctgtgaccttctttggtctgatcctagtaaagatgtcaaaggctgggggatgaatgatcgtggtgtctcctacacttttggtgctgacaaagttgctgagtttttaataaagaatgatatggatttagtctgtcgtgcccaccaggttgtagaggatgggtatgagttctttgctgatagacagcttgtgactatgttttctgctccaaactactgtggtgaatttgacaatgcgggtgcattgatgagtgtcgacgaaagtttaatgtgctctttccaaattcttaagcctgtggatcggaggtcgcggttcttttga

SEQ ID NO: 66 - DNA, 939 bp, Arabidopsis thaliana
atggcgcaacaagggcagggaagcatggaccctgccgctctcgatgatatcattcgtcgtttgttggattacagaaaccctaagcctggtaccaaacaggctatgctcaacgagtctgagatccgtcagctttgcatcgtctctagagagatttttcttcaacagcctaaccttcttgagctcgaagctcctatcaagatctgcggtgatattcatggacaatactcagatctattgaggctgtttgagtatggaggcttcccacctacagctaactatttattcttaggagattacgtagacaggggcaagcagagtttggagactatttgtcttctgcttgcttacaaaatcaaataccctgagaacttttttcttctaagaggaaaccacgaatgtgcttctattaacagaatctatggattctatgatgaatgtaaacgtcgattcagtgtgagactctggaaagtgtttacagattcttttaactgtctccctgttgctgctgttatagacgataagatattatgtatgcacggtggcctttctcccgatttgaccaacgtggaacagattaagaacattaagcgacctactgatgttcctgactctggtttgctctgtgatctgctttggtctgatccaagcaaagatgtcaaaggttgggggatgaatgaccgtggagtctcttacacgtttggccctgacaaagtcgcggagtttctaataaagaatgatatggatctcatctgccgtgcccaccaggttgtagaggatggatatgagtttttcgccgatagacagcttgttacgatattttctgctcccaactactgtggtgaatttgacaatgctggtgctatgatgagtgttgatgaaagtttaatgtgctctttccaaattcttaagcctgctgatcgaaagccccggttcttatga

SEQ ID NO: 67 - DNA, 996 bp, Arabidopsis thaliana
atggatcctggtactttaaactcggtgatcaataggttgcttgaagctagagaaaaaccaggaaagattgttcagttgtctgaaacagagatcaaacagctctgtttcgtctctagagatatcttcttgagacaaccaaatctcttggaacttgaagctcctgttaaaatatgtggggacattcatggacaatatccggatctcttgagactattcgaacatggcggataccctcctaattcaaactacttgtttcttggagattatgtcgatcgcggcaagcaaagcctcgaaacgatttgtcttttacttgcttacaagattaagttccctgaaaacttcttccttctcagaggaaaccatgaaagtgcatcaatcaatcgtatttacggcttctatgacgagtgtaaacgtagattcagtgtcaagatttggcgaatcttcactgattgcttcaactgtctccccgtcgctgcactcatcgatgagcggattttttgtatgcatggtgggctctccccggagctgctaagcttgaggcagattagggatattcgtcgtccaacggatattcctgatcgtggtttactctgtgatctcttgtggtctgatcctgataaagatgttagaggttgggggcctaacgatcgcggagtttcttacacttttggatcagatatagtttctggatttcttaaaagactcgatcttgacctcatttgtagggctcaccaggttgttgaagatggattcgagttctttgcgaataagcagctcgtaacgatattctctgcgccgaattactgtggggaatttgacaatgcaggtgcgatgatgagtgtgtctgaggatttgacctgctcttttcagatcttaaaatctaatgacaagaaatcaaagttcagtttcggaagcagaggtggtgctaaaactagcttcccttatcctaaagtgaagtcgattctgagttcgcaaaattcgaaagaatacaactga

SEQ ID NO: 68 - DNA, 993 bp, Glycine max
atggaacgtggggttcttgatagtatcattaataggctgcttgaagtcagaggcagaccagggaagcaggtgcaactttctgaggctgagatcaagcagctttgtcttgtctccagggatatcttcttgaggcagcccaacctcttggaactcgaggcaccaattaagatctgtggagatgtccatggtcaatattccgaccttctgcgactttttgagtatggtggattgcctcctcgttctaattacttgtttctaggggactatgttgatcgtgggaagcaaagtttggagacaatatgtctccttctggcttataaaataaaatatcccaacaactttttccttctgaggggcaaccatgaatgtgcttctataaaccgcatctacggattttatgatgagtgtaaacgaaggtacaatgtgaggctttggaaagtgttcacggaatgtttcaactgcttgccagtggcagctctaattgatgaaaagatactttgtatgcatggtggactctctcctgagttacacaatctgaatcagataaagagtttgccgcgtcctattgaagtgcctgaaactggtctactatgtgacctcctttggtctgatcctagtagtgacattcggggttggggagagaatgaccgcggagtttcctatacttttggtgctgatagggtgacagaattccttcagaagcatgatcttgatttgatttgcagggcccatcaggtcatggaagacggatatgaattctttgctaacaggcaacttgtaaccatcttctcggctcctaattactgtggagagtttgacaatgctggtgctatgatgactgttgatgagacacttgtttgctcttttcaaatattgaagcctgtagaaaataaaaagcctaataagtttgcctttgggagcacaactacagttaagcatagtactccaacaaaaaccaagttccagcaatcattttttggtgctaaagcatga

SEQ ID NO: 69 - DNA, 990 bp, Glycine max
atggaacgtggggttattgataatatcatcaataggcttctccaagtgagaggcagaccagggaagcaggtgcaactttctgaggctgagatcaagcagctttgtcttgtctccagggatatatttatgaggcagcccaacctcttggaactcgaggcaccaattaagatatgtggagatatccacggtcaatattccgaccttctgcgactttttgagtatggtggattgcctcctcgttataactacttatttttaggggactatgttgatcgtgggaagcaaagtttggagacaatatgtctccttctgtcttataaaataaaatatcccaacaactttttccttctgaggggaaaccatgaatgtgcttctataaaccgcatttacggattttatgatgagtgtaaacgaaggtacaatgtgaggctttggaaagtgttcacagagtgtttcaactgcttgccagtggcagctctaattgatgaaaagatactttgtatgcatggtggactctctcctgagttacacaatttaaatcagataaagggtttgccacgtcctattgaagtacctgaaactggtctactatgtgatctcctttggtctgatcctagtagtgacattcggggttggggagaaaatgaacgcggagtttcctatacttttggtgctgacagggtgacagaattccttcagaagcatgatcttgatttgatttgcagggcgcatcaggttgtagaagacggatatgaattctttgctaacagacaacttgtaactatcttctcggcacctaattactgtggagagtttgacaatgctggtgctatgatgactgttgatgagacacttgtgtgctcttttcaaatattgaagccagtagaaaataaaaagcctagtaagtttggctttgggagcacaactacagttaagcagagtacaacaaaagccaagttccagcaatcattttttggtgctaaagcatga

SEQ ID NO: 70 - DNA, 906 bp, Glycine max
atggaacgtggggttcttgatggtatcatcaataggctgcttcaagtcagagggagacctgggaaacaggttcagctttcagaggcagaaatcaggcaactttgtgcggtttccagagatatctttttgaagcagcccaatctattggaacttgaggccccaattaagatatgcggagacatccatggtcaatattctgaccttctgagactatttgagcatggtggatttcctcctcgctccaactacttattcttaggcgattatgtcgatcgtggaaagcaaagccttgaaactatgtgtcttcttttggcctacaagataaaataccctgagaacttcttccttcttagaggcaaccatgaatgtgcttctgtaaaccgcgtatacggcttctatgacgaatgcaaacgaagattcaatgtcaggctgtggaaaatattcgcagattgcttcaactgcatgcctgtggcagctatcattgaggaaaagattttctgtatgcatggtggactctctcctgaattgcacaatctaagtcagattagtagtttgccgcgtccaacagaagtacctgagagtggtctactatgtgatctcttgtggtctgatcctagtaaagacattgaaggttggggagagaacgaccgtggagtttcatatacttttggtgctagcagggtcacagaatttcttggaaagcatgatctagacttgatttgtagagcacaccaggttgttgaagatggatatgagttttttgccaatagacaacttgtgactatcttttctgcacctaactactgtggagaatttgacaatgctggagcaatgatgagtgttgatgagacactcatgtgctcttttcagatactaaggcctgcggaacacagaaagcctaaataa

SEQ ID NO: 71 - DNA, 960 bp, Glycine max
atggaacgtggggttcttgatggtatcatcagtcggctgcttcaagtcagagtgagacctgggaaacaggttcagctttcagaggcagaaatcaggcagctttgtgcggtttccagagatatctttttgaagcagcccaatctgttggaactggaaccaccaattaagatatgtggagacatccatggtcaatattctgaccttctgagactttttgagcatggtggattgcctcctcgctccaactacttattcttaggcgattatgtcgaccgtggaaagcaaagccttgagacaatatgtcttcttttggcatacaagataaagtaccctgagaacttcttcctccttagaggcaatcatgaatgtgcttccataaaccgcgtctacggcttctacgacgaatgtaaacggcgattcaacgtcaggctatggaaaatatttgcagattgcttcaactgcatgcctgtggcagctatcattgaggaaaagattttctgtatgcatggtggactctctccagaattgcacaatctaagtcagataagtagtttgccgcgtccaaccgaagtacccgagagtggtctactatgtgatctcttgtggtctgatcctagtaaagacattgagggttggggagacaatgagcgcggagtttcttatacttttggtgctagcagggtgacagaatttcttggaaagcatgatcttgacctgatttgtagagcacaccaggttgttgaagatggatatgaatttttttccaatagacaacttgtgactatcttttctgcacctaactactgtggagaatttgacaatgctggagcaatgatgactgttgatgagacacttatgtgctcttttcagatactaaggcctgtggaacacagaaagcctaagtttggttttgggagcaaaactacattcaaggcagttcttgatgctgctagagtatga

SEQ ID NO: 72 - DNA, 966 bp, Glycine max
atggacgagaacctgctcgacgatattatccggaggctcgtcgcggcgaagaacggtagaacgacgaagcaggtgcagcttacggaggcagaaattcgacaactctgtgtttcctccaaagagatctttctcagtcaacctaatcttctcgaactcgaggctcctattaagatttgtggagatgttcatggccaatactcagatcttttaaggttgttcgaatatgggggatacccacctgaagcaaattatttatttctcggagattatgttgatcgtggtaagcagagcatagagacaatatgtttactcctcgcctacaagatcaaatacaaggagaatttctttcttctcaggggcaaccatgagtgtgcatccatcaatcgtatatatggtttctatgatgagtgtaagaggaggtttaatgttcgcctctggaagacatttactgattgctttaattgccttcctgtcgctgctctgatagatgagaagatcctttgcatgcatggtggactatcgcctgatcttaaacacttggatcagattcgaagtattgctcgtcctattgatgtgccagatcatggccttctatgtgaccttctatgggctgatcctgataaagatcttgatgggtggggagaaaatgaccgaggtgtatcatttacatttggggctgacaccgttgttgaatttcttgaacatcatgatctcgatctgatttgcagagctcaccaggttgtagaggatggatatgagttttttgccaagcgtcagctggtgacgatattctccgcaccaaattactgcggtgaatttgataatgctggtgctatgatgagtgtggatgacaccttgacatgctctttccaaatacttaaatcttctgaaaagaaagggaaaggtggatttggcatcaacacatcaagaccaggaactccaccccataagggtgggaagaattaa

SEQ ID NO: 73 - DNA, 966 bp, Glycine max
atggacgagaacgtgctcgacgatattatccggaggctcctcgcggcgaagaacggtagaacgacgaagcaggtgctgcttaccgaggcggaaattcgtcaactctgtgtttcctccaaagagatctttctcagccaacctaatcttctcgaactcgaggctcctattaagatttgtggagatgttcatggccaatactcagatcttttaaggctgttcgaatatgggggatacccgcctgaagcaaattatttatttctcggagattatgttgatcgtggtaagcagagcatagagacaatatgcttactcctcgcatacaagatcaaatacaaggagaatttctttcttctcaggggcaaccatgagtgtgcatccatcaatcgtatatatggtttctatgatgagtgtaagaggaggtttaatattcgcctctggaagacatttactgattgttttaattgcctacctgttgctgctctggtagatgagaagatcctttgcatgcatggcggactatcgcctgatcttaagcacttggatcagattcggagtattgctcgtcctattgatgtgccagatcatggccttctctgtgaccttctatgggctgatcccgataaagatctagatgggtggggagaaaatgaccgaggtgtgtcatttacatttggggctgataaggttgctgagtttcttgaacatcatgatctcgatctgatttgccgagctcaccaggttgtagaggatggatacgagttttttgccaagcgtcagctagtgactatattctccgcaccaaactactgtggtgaatttgataatgctggtgctatgatgagtgtggatgacaccttgacatgctctttccaaatacttaaatcttctgaaaagaaagggaaatgtggatttggcaacaacacatcaagaccaggaactccaccacataagggtgggaagaattaa

SEQ ID NO: 74 - DNA, 969 bp, Arabidopsis thaliana
atggatgagactttacttgacgatataatacgacggcttttggcgacgaataatggaaggacggtgaagcaagcacagattactgagacggagatacgtcagctatgtttagcttcaaaagaggtttttctcagtcagcctaatctcctcgagctcgaggctcctatcaagatttgcggagatgttcatggtcagtttccagacctcttgcggttgtttgagtatggtggttatccaccagctgcgaactacttgtttcttggggattatgttgatcgtggtaagcagagcatagagacgatatgccttcttcttgcctataaggtcaaatacaagttcaacttctttcttctcagaggcaatcacgaatgtgcttcaatcaaccgtgtatatggattctacgatgaatgcaaaagaagatataatgttcgcttatggaaaacattcaccgagtgcttcaactgtctgcctgtttctgctctcattgatgataagatcctctgcatgcatggtggactatcgcctgatattaagagcttagatgacatcaggagaattcctcgtcctattgacgttcctgatcagggcattctttgtgatttgttgtgggctgatcctgacagagagattcaaggctggggggagaatgacagaggtgtctcttatacatttggggctgacaaagtagctgagttccttcaaactcatgaccttgatcttatatgccgagctcatcaggttgtagaagatggatacgagttctttgcaaagagacaactagtgacaatattctctgcacccaattactgtggcgagtttgacaatgctggtgcattgatgagtgttgatgatagcttaacatgttcattccaaatccttaaggcatctgagaagaaaggaagatttggattcaacaacaatgttcctagaccaggaaccccacctcataagggtggaaaaggtcgttaa

SEQ ID NO: 75 - DNA, 969 bp, Arabidopsis thaliana
atggaagatagtgtggtggatgatgttataaagaggttactaggagcgaaaaacgggaagacgacgaagcaggttcagttgacggaggctgagattaaacatctttgttctaccgctaagcagatctttctcactcaacctaatcttcttgaactcgaggctcctattaagatttgtggagatactcatggtcagttctcagatcttttgagattgttcgagtatggtggctacccaccagctgcaaactatttgtttcttggggattatgtagatcgtggaaagcagagtgtagagaccatatgccttcttcttgcctacaagatcaaatacaaagagaatttctttcttctacgtgggaaccatgagtgtgcttctataaaccgtatatacggattctatgacgagtgcaagaagagatatagcgttcgagtgtggaagatatttactgactgctttaattgtctacctgttgctgctcttattgatgaaaagatcctctgtatgcatggtgggctatcgcctgagttgaagcacttggatgagatcaggaacattcctcgtcctgctgacattcctgatcatggactactgtgtgatctgttgtggtctgatcctgacaaagacattgaaggatggggagagaatgacaggggtgtctcgtacacttttggggctgataaagtcgaggagtttcttcaaacgcatgaccttgacttaatttgtagagctcatcaggttgtggaagacgggtatgaattctttgcaaatagacagctagtaacgatattctcggccccaaactactgcggagagtttgataacgcaggagcaatgatgagtgtggatgattccttgacgtgctcatttcagatccttaaagcatctgagaagaaaggaaacttcgggtttggcaaaaatgcaggaaggcgaggaaccccgcctcgcaagggtggtggaaaaggctga

SEQ ID NO: 76 - DNA, 915 bp, Zea mays
atggacaggggaacggtggaggacctcatacggcggttgctggacgggaagaagcacaaggcgacggggaagaaggtgcagctgaccgagaccgagatccggcacctctgcgtcaccgccaaggagatcttcctctcccagcccaacctcctcgagctggtggcccccatcaacgtctgcggtgacatccacgggcagttctcggacctgctgcggctcttcgagtacggcggcctgccgccgacggccaactatctcttcctcggcgactacgtggaccgcggcaagcagagcatcgagaccatctgcctgctgctggcgtacaagatccggtacccggacaacttcttcctgctccggggcaaccacgagtgcgcctccatcaaccgcatctacggcttctacgacgagtgcaagcgccgcttcagcgtccgcctctggaagatcttcaccgactgcttcaactgcctgcccgtcgccgccgtcatcgatgacaagatactatgcatgcacggcgggctctccccggacctggacaacctcaaccggatcagggagatccagcgccccgtcgacgtccccgaccagggcctcctctgcgacctcctgtggtccgacccggaccgcgacagctccggatggggcgacaacgaccgcggcgtctccttcaccttcggcgccgacaaggtcaccgagttcttaaacaagcacgacctcgaccttgtctgccgcgcccaccaggtcgtggaggacggctacgagttcttcgccgaccggcagctggtgaccatattctcggcgcccaactactgcggcgagttcaacaacgccggcgcgctgatgaacgtcgacgccagcctgctctgctcgttccagatcctcaagccgtacaggggcaaagcacaaacggagtga

SEQ ID NO: 77 - DNA, 918 bp, Sorghum bicolor
atggacgggggcacggtggaggatctcatacggcggctgctggacggcaagaagcacaaggtgacggggaagaaggcggtgcagctgacggagcccgagatccggcacctctgcgtcaccgccaaggaggtcttcctctcccagcccaacctcctggagctggaggcccccatcaacgtctgcggtgacatccacgggcagttctcggacctgctgcggctgttcgagtacggcggcctgccgccgacggccaactacctgttcctcggcgactacgtggaccgcgggaagcagagcatcgagaccatctgcctgctgctggcgtacaagatccggtacccggacaacttcttcctgctgcggggcaaccacgagtgcgcctccatcaaccgcatctacggcttctacgacgagtgcaagcgccgcttcagcgtccgcctctggaagctcttcaccgactgcttcaactgcctgcccgtcgccgccgtcatcgacgacaagatcctctgcatgcacggcggcctctccccggacctcgacaacctcaaccggatcagggagatccagcgccccgtcgacgtccccgaccagggcctcctctgcgacctcctctggtccgaccccgaccgcgacagctccggctggggcgacaacgaccgcggcgtctccttcaccttcggcgccgacaaggtcaccgagttcttgaacaagcaggacctcgacctcgtctgccgcgcccaccaggtcgtggaggacgggtacgagttcttcgccgaccggcagctggtgaccatattctcggcgcccaactactgcggcgagttcaacaacgccggcgcgctgatgaacgtcgacgccagcctgctctgctcgttccagatcctcaagccgtacaggggcaaagcacagacggagtga

SEQ ID NO: 78 - DNA, 924 bp, Oryza sativa
atgatggacgggaacgcggtggacgagctgatacggcggctgctcgacgggaagaaggtcaagccgtcgtcgtcggcgaagaaggtgcagctcagcgaggcggagatccggcagctctgcgtcaccggcaaggacatcttcctctcccagcccaacctcctcgagctcgaggcccccatcaacgtctgcggcgacatccacggccaattctccgacctgctccggctgttcgagttcggcgggctgccgccgacggcgaactacctgttcctcggcgactacgtggaccgcgggaagcagagcatcgagacgatctgcctcctgctggcatacaagatcaagtacccggacaacttcttcctgctgcgaggcaaccacgagtgcgcgtcgatcaaccgaatctacgggttctacgacgagtgcaagcgccggttcagcgtccgcctctggaagctcttcaccgactgcttcaactgcctccccgtcgccgccgtcatcgacgacaagatcctctgcatgcacggcggcctctcgccggacctcgacagcctcgaccggatcagggagatcgcccgacccgtcgacgtccccgaccagggcctcctctgcgacctcctctggtccgaccccgaccgcgagagctccggctggggcgagaacgaccgcggcgtctccttcaccttcggcgccgacaaggtcaccgagttcctcaacaagcacgacctcgacctcatctgccgcgctcaccaggtggtggaggacggctacgagttcttcgcggacaggcagctggtgaccatattctcggcgccgaactactgcggcgagttcaacaacgccggcgcgctgatgaacgtggacgccagcctgctctgctcgttccagatcctcaagccgttcagaggcaagtcgcaggcggagtga

SEQ ID NO: 79 - DNA, 909 bp, Glycine max
atggaaggattggatggtttgatagagaggcttttggaagtaaggaagaacagaggcaaacaaattcaactggtagagtcagaaattcgcagcctttgcagcacggcaaaagatctcttcctcaggcagccaaacctgttggaattggaagctcccattaacgtttgtggtgatatacacgggcaatatccagacctgttgcgagtgttggagtacggtgggtttccgccggattcgaattacctattccttggagactacgtggacagaggaaaacagagcgtggaaacaatatgcctgctgctcgcctacaagataaaatacccagaaaactttttccttctccgaggaaaccacgagtgtgcctctattaacagaatctacgggttttacgacgagtgcaagcgcagattcagcgtccgtctctggaagatattcacagattgcttcaactgtttgcccgtggccgcagtgatcgatgacaaaatcctctgcatgcacggcgggctttccccggacatggagagcctgaatcagatcaaagccatagagaggcccgtggacgtgcccgaccagggcctcttatgcgacctcctctgggccgatcccgacaacgaaatcagtggctggggcgagaatgataggggcgtctcctacactttcggccccgataaggtctccgagttcttgaagaaacacgatctcgatctcatttgcagggcccaccaggtcgtcgaagatggctaccaattcttcgctgacaggcagctcgttaccatcttctctgccccaaactattgcggagagttcaacaatgccggtgccctcatgtgcgtcgaccaaactttactctgttcctttcagattgtcaaaccctttggcacctttagaggaaaactcacttcttaa

SEQ ID NO: 80 - DNA, 969 bp, Oryza sativa
atggatgcggcggcgctcgacgacttgatccggcggctcctggacgcccgcggcggccgcacggcgcgccccgcgcagctcgccgacgcggagatccgcaagctctgcgccgccgccaaggacgtcttcctctcccagcccaacctcctcgagctcgaggcccccatcaaaatctgcggagatatccatggccagtattctgatcttcttcggttatttgagtatgggggcttcccaccagaggcaaactacttgtttctaggtgattatgttgaccgtgggaaacagagtatagaaactatatgtcttctccttgcgtataagattaagtacccagagaacttcttcctccttaggggaaaccatgagtgcgcatctatcaaccgaatctacggattctttgatgaatgcaaaaggaggtttaatgttcgtatttggaaggtttttactgattgtttcaactgcctccctgtggctgcacttattgatgacaagatcctatgcatgcatgggggtttatctcctgacctaaagaacatggaccagattcgcaacattgcccgcccagttgatgttccagaccatggtctcctgtgtgatctgttgtggtcagacccagataaagaaatcgaaggttggggtgagaatgacaggggtgtatcatacacttttggagctgataaggtcgctgagtttctccagacacatgatctagatctgatctgcagggctcaccaggttgtggaggatggctatgaattctttgctaagcgccagcttgtaaccatattctctgcacccaactattgtggcgagtttgacaatgcgggggcgatgatgagcattgatgattctcttacatgctcattccagatccttaagccttctgataagaaaggaaaagcaggaaccggaaatatgtcaaaacctggaactccaccaaggaagataaaaattaatattatttag

SEQ ID NO: 81 - DNA, 975 bp, Zea mays
atggacgaggcggcggtcgacgacctcatccggcggctcctcgaggcccgcggcgggcgcaccccgcgcaacgcgcaggtgaccgacgctgagatccggcgcctctgcgccgccgccaaggatgtgttcctctcccagcccaacctcctggagctcgaggcccccatcaagatatgcggggatgtccatggtcagtattcagaccttcttcgattatttgagtatggtggctatccaccagatgcaaattatctgttcctcggtgactatgttgatagggggaaacagagcatagaaacaatatgccttctcttggcatacaagataaagtacccagaaaacttctttcttctcaggggaaaccatgaatgcgcctcaatcaaccgaatatatgggttttttgatgagtgtaagaggagattcaatgttcgtatctggaagatattcaccgagtgttttaactgcctcccagtggctgcgcttattgatgacaagatcttttgcatgcatggagggttgtctcctgagctaaagagcatggaccaaatacgtaacatttctcgtcctgtggatgttcctgatgttggtctcctatgtgatctgttatggtcggaccctgataaggagattgataggtggggtgagaatgacaggggtgtttcctacacctttggagctgacgtagttgccgagtttcttcagaaacatgatctagatttgatctgcagggcccaccaggtcgtagaggatggttatgaatttttcgcaaaacgccagcttgtaacaatattctcagcaccgaactactgtggagagttcgacaatgctggtgcactgatgagcattgacaactccctagtatgctcgttccagatactcaagccttctgagaagaaaggaaaagcaggaaatgggaacatgccaaagcccggtacacctcccaggaagataaaaataagtgttacccatatttag

SEQ ID NO: 82 - DNA, 975 bp, Sorghum bicolor
atggacgaggcggcggtcgacgacttgatccggcggctcctcgaggcccgcggcgggcgcaccccgcggaacgcgcaggtgaccgacgccgacatccggcgcctctgcgccgccgccaaggatgtgttcctccaacaacccaacctcctggagctcgaggcccccatcaagatatgcggggatgtccatggacaatattcagatcttcttcgattatttgagtatggtggctatccaccagacgcaaattatctgttcttgggtgactatgttgatagggggaaacagagcatagaaacaatatgccttctgttggcgtacaagttaaagtatccagaaaacttcttcctcctcaggggaaaccatgaatgcgcctcgatcaaccggatatatgggttctttgatgagtgcaagaggagattcaatgtccgtatctggaagatattcactgagtgctttaattgcctccctgtggctgcgcttattgatgataagatcttttgcatgcatggggggttgtctcctgaacttaagaacatggatcagatacgtaacatttctcgtccggtggatgttcctgacgttggtctcctttgtgatttgctatggtcggaccccgagaaagagcttgatgggtggggtgagaatgacaggggtgtttcctacacatttggagctgatatagttgccgagttccttcagaaacatgatctagatttgatctgcagggcccaccaggttgtggaggatgggtatgaattttttgcaaaccgccagcttgtaacaatattctcagcaccaaactactgtggagagttcgacaatgctggtgcactgatgagcattgatgactccctagtatgttcgttccagattctcaagccttcagaaaagaaaggaaaagcaggaacttccaacatgtcaaaacctggaacacctcccaggaagataaaaataagtgttactcgaatttag

SEQ ID NO: 83 - DNA, 978 bp, Zea mays
atgatggatccggccttgcttgacgacgtcatacgccggctgctggaggtgaagaatctcaagcctgggaagaacgcgcaggtgactgacgccgagatccggcgcctctgcgccaccgccaaggacgttttcctctcccagcccaacctcctggagctcgaggcccccattaagatatgcggggatgtccatggccagtattcagacctccttcgattatttgagtatggtggctatccaccagatgcaaattatctgttcctgggtgactatgttgatcgggggaaacagagcatagaaacaatatgccttctcttggcgtacaagataaagtacccagaaaacttctttcttctcaggggaaaccatgaatgtgcctcgatcaaccggatatatgggttttttgatgagtgtaagaggagattcaatgtgcgtatctggaagatattcactgagtgttttaactgcctcccagtggctgcacttattgacgataagatcttttgcatgcatggggggttgtctcctgagctaaagaacatggatcaaatacgtaacatttctcgtcctgtggatgttcctgacgttggtcttctatgtgatctgttatggtcggaccctgataaagagattgttgggtggggtgagaatgacaggggtgtttcctacacctttggagctgacatagttgctgagtttcttcagaaacatgatctagatttgatctgcagggctcaccaggtcgtagaggatggctatgagtttttcgcaaatcgtcggcttgtaacaatattctcagcaccgaactactgtggagagtttgacaatgctggtgcactgatgagcatcgacgactccctagtatgctcgttccagatactcaagccttctgaaaagaaaagaaaagcaggagctgagaacatgccaaagcccggtacacctcccaggaagataaaaataagtgttacccgcatttag

SEQ ID NO: 84 - DNA, 975 bp, Zea mays
atggacgaggcggcgattgacgacttgatccggcggctcctcgaggcccgcggcgggcgcaccccgcgcaacgcgcaggtgactgacgccgagatccggcgcctctgcgccaccgccaaggacgttttcctctcccagcccaacctcctggagctcgaggcccccattaagatatgcggggatgtccatggccagtattcagacctccttcgattatttgagtatggtggctatccaccagatgcaaattatctgttcctgggtgactatgttgatcgggggaaacagagcatagaaacaatatgccttctcttggcgtacaagataaagtacccagaaaacttctttcttctcaggggaaaccatgaatgtgcctcgatcaaccggatatatgggttttttgatgagtgtaagaggagattcaatgtgcgtatctggaagatattcactgagtgttttaactgcctcccagtggctgcacttattgacgataagatcttttgcatgcatggggggttgtctcctgagctaaagaacatggatcaaatacgtaacatttctcgtcctgtggatgttcctgacgttggtcttctatgtgatctgttatggtcggaccctgataaagagattgttgggtggggtgagaatgacaggggtgtttcctacacctttggagctgacatagttgctgagtttcttcagaaacatgatctagatttgatctgcagggctcaccaggtcgtagaggatggctatgagtttttcgcaaatcgccagcttgtaacaatattctcagcaccgaactactgtggagagtttgacaatgctggtgcactgatgagcatcgacgactccctagtatgctcgttccagatactcaagccttctgaaaagaaaagaaaagcaggagctgagaacatgccaaagcccggtacacctcccaggaagataaaaataagtgttacccgcatttag

SEQ ID NO: 85 - DNA, 957 bp, Arabidopsis thaliana
atgatgacgagtatggaagggatgatggagatgggtgtattggatgatattataaggagattgttggaaggtaaaggaggtaaacaggttcagctttcggaggtcgagatccgtcaactttgcgttaacgccagacaaatcttcctttctcagcctaatcttctcgagcttcatgcccccattcgcatctgcggtgatatccatggacagtaccaagatcttctgaggctattcgaatacggaggctatcctccttcagcgaactatctcttcctcggcgattacgttgatagaggcaagcaaagtcttgaaacaatatgtttactcttggcttacaaaatccggtacccatccaagatatttctcttgagaggaaaccatgaagatgctaagatcaacaggatttacgggttctatgacgaatgcaaacggaggtttaatgtacggctttggaagatattcaccgattgcttcaactgtttgccggtagctgctctcattgacgagaagatcctatgtatgcatggtggattgtcaccggaattggaaaacttggggcagattcgggagattcaaaggcctacagagattccagacaatggtcttctttgtgatttactttggtctgatcctgatcagaagaatgaagggtggactgatagcgatcgaggtatctcctgcacgtttggagccgatgtagtcgctgactttttggataagaatgatctcgatctcatttgcagaggccatcaggtagtggaagatgggtatgagtttttcgcaaaacggaggttagtcacaatattctcagctccaaactatggaggagagtttgacaatgccggtgcattattgagcgtggatcaatctctcgtttgctcctttgagattctgaaaccggctccagcttctagcactaatcctctcaagaaggtaccgaaaatggggaagtcttga

SEQ ID NO: 86 - DNA, 975 bp, Arabidopsis thaliana
atgatgacgagtatggaagggatggtggagaaaggagtattggatgatattataagaagattgttagaaggtaaaggaggcaaacaggttcagctttccgagagcgagattcgtcaactctgctttaacgctcgtcaaatcttcctctctcaacctaatctccttgatctccatgccccaattcgcatctgcggtgatattcatggtcaatatcaagatcttttgaggttgtttgaatacggaggttatcctccttcagcaaactatctattccttggtgattacgttgacagaggcaaacaaagtcttgagaccatatgtttgcttcttgcttacaagatacggtacccatcgaagatatatctgttgagagggaaccatgaggatgctaagatcaacaggatttacgggttttatgacgagtgcaaacggagattcaatgtacgactctggaaggtgtttactgattgcttcaactgtttacctgtagctgcacttattgatgagaagatactgtgtatgcacggtggtttgtcaccagatttggataatttgaatcagattcgagagattcaaaggcctattgagattccagacagtggtcttctttgtgatttactttggtcagatcctgatcagaagattgaaggttgggctgatagtgatcgaggtatctcttgcacttttggagctgataaagtcgctgagttcttggataagaatgatcttgacctcatttgccgaggccatcaggtagtagaagacgggtatgagtttttcgcaaaacggagattagtcacgatattctcagctccaaactatggtggggagtttgacaacgctggtgcgttattaagcgttgacgagtctcttgtatgttcctttgagattatgaaaccagccccagcttcaagcagtcatcctctcaagaaggactttcacaacagaaccttaggttataatttgagtgcttag

SEQ ID NO: 87 - DNA, 1128 bp, Glycine max
atgttacggttgggccatcttgtccatctatccaaatttccccatcccgcaactaatttatttatttatttttcctttatttatcttctttctctggctcgagcatccaaaagggttgttttttttttttataagaagaagaggagagggaagatgatgatgatgacaatggaggggatgatggacaagggggtgttggatgatgtgataagaaggcttctggaagggaaaggagggaaacaggtgcaactctctgagtcggagatccgtcaactctgcgtcaacgccagacaaatcttcctctctcagccaattcttctcgacctccgtgctcccatccgcatctgcggtgatatacatggtcagtaccaggacttgctgaggctttttgaatatggtggctaccctcctgcggcaaactacttgtttctgggggattatgtggatagagggaagcaaagtttggagactatatgtttgcttttggcctacaaaataagatatccagacaaaatttatctcttgaggggaaaccatgaagaagcaaagataaaccgtatatatggtttttatgatgaatgtaaaaggaggttcaatgttaggctatggaagatatttacggactgctttaactgtttgccagtggctgcactcattgatgagaaaatactttgtatgcatgggggactctctccagaattagaaaatttagatcagataagagagattcaaaggcctactgaaattccagatagtggtctcctatgtgatctgctttggtctgatcctgatgctagcattgagggctgggcagagagtgaccgaggagtttcatgtacttttggagctgatgtagttgcagagtttttggataaaaatgacgttgatcttgtttgcagagggcatcaggttgtggaggatggatatgagttttttgctaaaagaagattagtcacgatattttctgctccaaattatggtggggaatttgacaatgctggtgcgttgttaagcgttgatgattctcttgtatgttcctttgagatactgaaacccgctgatagagcatcaggaagtggttcatcaaaaatgaattttaagaagccacccaaactcggaaagatatag

SEQ ID NO: 88 - DNA, 975 bp, Glycine max
atgatgatgatgacaatggaggggatgatggacaagggggtgttggatgatgtgataagaaggcttctggaagggaaaggagggaaacaggtgcagctctctgagtccgagatccgtcaactctgcgtcaacgccagacaaatcttcctctctcagccaattcttctcgaccttcgtgctcccatccgcgtctgcggtgatatacatggtcagtaccaggacttgctgaggctttttgaatatggtggctaccctcctgcggcaaactacttgtttctgggggattatgtggatagagggaagcaaagtttggagactatatgtttgcttttggcctacaaaataagatatccagacaaaatttatctcttgaggggaaaccatgaagaagcaaagataaaccgtatatatggtttttatgatgaatgtaaaaggaggttcaatgttaggctatggaagatatttacagactgctttaactgtttgccagtggctgcactcattgacgagaaaatactttgtatgcatgggggactctctccagaattacaaaatttagatcaaataagggagattcaaaggcctactgaaattccagataatggtctcctatgtgatctgctttggtctgatcctgatgctagcattgagggctgggcagagagtgaccgaggagtttcatgtacttttggagctgatgtagttgcagagtttttggataaaaatgaccttgatcttgtttgcagagggcatcaggttgtggaggatggatatgagttttttgctaaaagaagattagtcacgatattttctgctccaaattacggtggggaatttgacaatgctggtgcattgttaagcgttgatgattctcttgtatgttcctttgagatattgaaacccgctgatagagcttcaggaagtggttcatcaaaaatgaattttaagaagccacccaaacttggaaagatatag

SEQ ID NO: 89 - DNA, 978 bp, Oryza sativa
atgatgatgacgcgggcgtcgatgggggcgatggaaggcgcggcggtggacgaggtggtgcggcgcctcgtggagggcgggcgcggggggaggcaggtgcagctgtcggaggcggagatccggcagctctgcgtcgaggcgaagcgggtgctcctgtcgcagccgaacctcctgcgcatccacgcccccgtcaagatctgcggtgatatccatgggcaatttgttgatcttctgaggctgtttgatttgggcggttatcctccaacttccacttatcttttccttggagattatgtggacagaggcaaacaaagcttggaaaccatatgcttgcttctggcatacaaagtgaagtatcctgataagattttcctgttaaggggaaaccatgaagatgcgaaaattaacagagtgtatggtttctatgatgaatgtaagaggcgattcaatgttcgactgtggaagattttctgtgattgcttcaactgcttgcctatggcagcactcattgacgataagatactctgcatgcatggtggcctctcacctgaactgaatagcttggatcaaataaaggatatcgagaggcccactgagattcctgactatggtcttttgtgtgatttagtttggtctgatcctagtcctgactcagaagggtggggagagagtgacagaggtgtttcctgcacttttggtgcagataagcttgtagaatttttggagaagaatgatcttgatcttatatgccgcgctcatcaggttgttgaggatggctacgagttctttgcgcaaaggagattagtgacaatcttttcagctccaaactattgtggggaatttgataatgcgggtgctctgttgagcatagatgaaagtttaatgtgttcttttcaaatcttgaagccaaatgatacaggggctccacattcaaggaaaccaacttcaaacaagaccccaaaaactggaaacgcttaa

SEQ ID NO: 90 - DNA, 978 bp, Sorghum bicolor
atgatgatgacgcgggcgtcgatgggggcgatggatgcggcggcggtggacgaggtggtgcgtcgcctcgtcgagggcggccgcgggggccggcaggtgcagctgtccgaggccgagatccgccagctctgcgtcgaggccaaacaggtgctgctgtcgcagccaaacctcctgcgcatccacgcgcccgtcaagatctgcggtgatatccatggacagtttgttgatctcttgaggcttttcgatttgggtggttatcctccaacttcaacttatattttccttggagactatgtggatagaggcaaacaaagcttagaaaccatatgtctgcttctggcatacaagttgaagtaccctgataatatataccttttaaggggaaaccatgaagatgccaaaattaacagagtttatggtttctatgatgaatgtaaaaggagatttaatgttcgactctggaagatattctgtgattgcttcaactgcttgcctatgtcagcacttattgatgacaaagtattatgcatgcatggtggcctctcaccagagttgaatagcttggatcaaataaaagatattgagaggcctactgaaattcctgactatggtcttctgtgcgatctcctttggtctgatcctagtcatgatacagaaggatggggggagagtgatagaggtgtttcttgcacttttggtgcagataagcttgtcgaatttttggataagaatgatctcgaccttgtgtgccgagctcatcaggtggtagaagatggctacgaattctttgcagaaagaagactagtgacgatattttcagctccaaactactgtggcgaatttgataatgcaggtgctctattgagcatagatgaaagcttgatgtgttcttttcagatcttgaaaccaaatgaaacaggtggaccacattcaaggaaacctcttccaagcaaggcaccaaaaggagagaatgtctaa

SEQ ID NO: 91 - DNA, 969 bp, Zea mays
atgatgatgacgcgggcaccgatggggccgatggagggcgcggcggtggacgagatggtgcgccgcctcgtggagggcggccgaggcgggcgccaggtgcagctgtccgaggcggagatccggcagctctgcgtagagggcaagcgggtgctcctctcccagcccaatctcctccgaatccacgccccagtcaagatctgcggtgatatccatggccagtttgtcgaccttctgagattattcgatttgggtggttatcctccagcttcaacttacgtattcctcggagactatgtcgatagaggcaaacagagcctggagactatatgcttgctgctggcgtacaaaatccggtaccccgaaaatattttcctgttgaggggtaaccacgaggatgcaaagatcaacagagtctatggtttttatgacgaatgtaagaggaggttcaatgttcggctgtggaagatattctctgattgcttcaactgcctgcccatcgcagcgctcatcgatgacaagatactgtgcatgcatggtggcctctcgcccgaattgactagtttggaccagataaaagatattgagcgacctgctgagattcctgattatggtctcctgtgtgatctgctttggtctgatcctagccccgatggagaagggtggggggagagtgacaggggtgtctcgtgtacgtttggtgcagataaacttgtagagtttttggagaagaatgatctcgatcttatatgccgagctcatcaggtggttgaagatggctatgagttctttgcacaacgaagattagtgacaatcttttcagctccaaattattgtggagagtttgataatgttggcgctcttttaagcatagatgagagcctaatgtgttcatttcagatcctgaagccaactgatatgggtccgccgcatgcaagaaaacaaattccaaataagccaacaagagggtga

SEQ ID NO: 92 - DNA, 1056 bp, Zea mays
atgatgatgacgcgggcaccgatggggccgatggagggcgcggcggtggacgagatggtgcgccgcctcgtggagggcggccgaggcgggcgccaggtgcagctgtccgaggcggagatccggcagctctgcgtagagggcaagcgggtgctcctctcccagcccaatctcctccgaatccacgccccagtcaagatctgcggtgatatccatggccagtttgtcgaccttctgagattattcgatttgggtggttatcctccagcttcaacttacgtattcctcggagactatgtcgatagaggcaaacagagcctggagactatatgcttgctgctggcgtacaaaatccggtaccccgaaaatattttcctgttgaggggtaaccacgaggatgcaaagatcaacagagtctatggtttttatgacgaatgtaagaggaggttcaatgttcggctgtggaagatattctctgattgcttcaactgcctgcccatcgcagcgctcatcgatgacaagatactgtgcatgcatggtggcctctcgcccgaattgactagtttggaccagataaaagatattgagcgacctgctgagattcctgattatggtctcctgtgtgatctgctttggtctgatcctagccccgatggagaagggtggggggagagtgacaggggtgtctcgtgtacgtttggtgcagataaacttgtagagtttttggagaagaatgatctcgatcttatatgccgagctcatcaggtggttgaagatggctatgagttctttgcacaacgaagattagtgacaatcttttcagctccaaattattgtggagagtttgataatgttggcgctcttttaagcatagatgagagcctaatgtgttcatttcagatcctgaagccaactgatatgggtccaccgcatgcaagaaaacaaattccaaataaggtaaatcagctaccttcacataggaatataggattaatattcagctataaagctacaacactagagatcacatgtttattttattattatcatgttagctag

SEQ ID NO: 93 - DNA, 969 bp, Zea mays
atgatgatgacgcgggccccgatgggaccgatggagggcgcggcggtggacgaggtggtgcgccgtctcgtggagggcggccgcggcggccgccaggtgcagctgtcagaggcggagatccggcagctctgcgtagacggaaagcgggtgctcctttcccagcccaacctcctccgaattcacgcccccgtcaagatctgcggtgatatccatggccagtttgttgatcttctgaggttgttcgatttgggtggttatcctccagcttcaacttatgtattccttggagactatgtcgatagaggcaaacagagcttggagactatatgcctgctgctggcatacaaaatcaggtatcccgaaaaaattttcctgttgagggggaaccatgaggatgcgaagatcaacagagtctatggtttctatgacgaatgtaagaggaggttcaatgttcggctgtggaagatattctcagattgcttcaactgcctgcctattgcagcgctcattgatgacaagatactgtgcatgcatggtggtctctcaccagaatggactagtttggatcagataaaggatattgagaggcctgctgagattcctgattatggtctcttgtgtgatctgctttggtctgatcctagtcctgatggagaagggtggggggagagtgacagaggtgtttcgtgtacgtttggtgcagataagctcgtagagtttttggagaagaacgatctcgaccttatatgccgagctcatcaggtagttgaagatggttatgagttttttgcacaacgaagattagtgacaatcttttcagctccaaattactgtggagagttcgataacgccggtgctctgttgagcatagatgagagcctaatgtgttcatttcagattctgaagccaactgatatgggtccaccacatgcaagaaaacaaattccaaataagccagcaagagggtga

SEQ ID NO: 94 - DNA, 969 bp, Sorghum bicolor
atgatgatgacgcgggccccgatggggccgatggaaggcgcggcggtggacgaggtggtgcgccgcctcgtggagggcggccgcggcgggcgccaggtgcagctgtccgaggcggagatccgccagctctgcgtcgagggcaagcgcgtgctcctctcccagcccaacctcctccgaatccacgcccccgtcaagatctgcggtgatatccacggccagtttgttgaccttctgaggttgttcgatttgggcggttatcctccagcttcgacttacgtattcctcggagactatgtcgatagaggcaaacagagcttggaaactatatgcttgctgctggcatacaaaatccggtaccccgagaagattttcctgttgagggggaaccatgaggatgccaagatcaacagagtctacggtttctatgacgaatgtaagaggaggttcaatgttcggctgtggaagatattctccgattgcttcaactgccttcctattgcagcgctcattgatgacaaaatactgtgcatgcatggtggtctgtcacctgagttgactagtttggaccagataaaggatattgagcggcctgctgagattcctgattatggtctcctgtgtgatctgctgtggtctgatcctagccctgatggagaagggtggggggagagtgacaggggtgtttcgtgtacgtttggtgcagataaacttgtggagtttttggagaagaacgatctcgatcttatatgccgagctcatcaggtggttgaagatggttatgagttctttgcacaacgaagattagtgacaatcttttcagctccaaattattgtggagagtttgataatgctggtgccctattgagcatagatgagagcctaatgtgttcatttcagatcctgaaaccaactgatatgggtccaccacatgcaagaaaacaaattccgaataagccagcaagagggtga

SEQ ID NO: 95 - protein, 15 aa, Artificial sequence
  motif
LXEVRXARPGKQVQL

SEQ ID NO: 96 - protein, 15 aa, Artificial sequence
  motif
GAMMSVDEXLMCSFQ

SEQ ID NO: 97 - DNA, 969 bp, Pennisetum glaucum
atggcggcggcgacggcggcgggagggcaaggcggcgggggcatggacaacgcgctcctcgacgacatcatccgccgcctgctcgaggtgcggaccgcgcgccccgggaagcaagtgcagctctccgaggcggagatccgccagctctgcaccgcctcccgcgaaatcttcctcagccagcccaacctcctcgagcttgaggcgcccatcaagatctgcggtgacatccatggtcaatacagtgatcttttaaggctttttgaatatggaggctttcctcctgaagccaactatctattcctaggtgattatgttgatcgaggcaaacaaagtctggaaactatatgccttcttcttgcatacaaaatcaagtaccctgagaacttttttcttctgaggggcaatcatgagtgtgcttcaataaacagaatatatggattctatgatgaatgcaagcgcagatttaatgtgcggctatggaaagtcttcactgaatgtttcaacacacttcctgtggctgctttaatagatgataaaatattatgtatgcatggtgggctctcacctgatctaacacacttggatgagataaagaacttgcaacgtccaactgatgtaccggatcaaggtctactgtgtgatttgctttggtcagatccaggaaaagatgttcaagggtggggcatgaatgatagaggggtctcatatacctttggtgctgacaaggtttcagaattcttggaaaagcacgatcttgatcttatttgtcgcgctcaccaggttgttgaggatgggtatgaattctttgctgacagacagctcgtcactatattctcagcccccaactactgtggtgaatttgataatgctggtgcgatgatgagtgttgatgaaactttgatgtgctcattccaaattctcaaacccgctgagagaaaaaacaaatttatggggtcaaacaaaatgtga

SEQ ID NO: 98 - DNA, 984 bp, Dennstaedtia punctilobula
atggaccccgctgttgttgatgatatcattgcccgcctcctcgaagtgcgaagttcccgccctggcaagcaggtgcagctctctgagaatgagatccggcagctctgctccacttccaagaccatcttcttacagcagcctaacttgctggagcttgaggcccccatcaagatttgtggggatattcatggacaatactctgatctcttgaggctttttgagtatggtggtttgcccccacaagcgaattacttatttctaggagattatgtggatcgaggcaagcaaagccttgaaacgatatgcctcctccttgcttacaaaatcaagtaccctgagaacttcttcctgctgaggggtaaccacgaatgtgcctcgatcaatcgaatatatggcttctatgatgaatgcaagaggcggttcaatgtaaggctgtggaaggcatttactgaatgcttcaactgtctaccagttgcagctcttattgatgagaagatactgtgcatgcatggcggcctctctccagaactaagtaatctggatcaaatcaagaatatctaccgccctactgatgtcccggatcaggggttgctatgcgatttgctatggtccgacccagacaaagaggtttcaggctggggggagaatgacagaggggtttcctacaccttcggcgttgacaaggtgattgagttcttggaaaaacacgacctggatctcgtttgccgggcacatcaggttgtcgaagaaggctacgagttttttgcggacagacgattggtgaccatcttctctgctccaaattactgcggcgagtttgacaatgctggggccatgatgagcgtggatgaatctctcatgtgctcgtttcagatcttgaagccagcggataaaaagccgaagttcaattataataattcaagtttggctcgggcaggaactccacctagaggagttaagccatttcaaggaggaaaagtttaa

SEQ ID NO: 99 - DNA, 975 bp, Dennstaedtia punctilobula
atggaccctgctgcgctggaggatatcatacatcgtcttttggaagtgcgggatgccaggcctggcaagcaggtgcagctctcggaggctgagatccgacaactctgtctcacctctaaggctatcttcatgcaacagcccaacctgctcgaactagaagctcccatcaagatctgtggtgacattcatggccagtattcagatctgttgcggctttttgaatacggtggactaccgccacaggcaaattatctttttttaggagattacgtggacaggggcaagcaaagtcttgaaaccatatgtctgttactagcctacgagatcaaatatcccgagaacttttttcttctgagaggaaatcatgaatgtgcgtctatcaatcgcatatatgggttttatgatgaatgcaagaggaggttcaacatcaggttgtggaagaccttcacggactgtttcaactgtctccctgtggcagctttgatagatgagaagattctttgcatgcatggtggcctttccccagaattgcacaaccttgaccaaatccgatcaatacctcgccccacagatgtacctgatcaagggctgctctgtgaccttttatgggcagatcctgagaaagagctgtctgggtggggcgagaatgatagaggcgtatcttttacttttggtgcagataaagttagtgagtttttacagaagcatgatctcgacttgatctgccgagcccatcaggttgtagaggatggatatgaattttttgctgacagacagcttgtgacaattttttcagccccaaattactgtggggagtttgataatgcaggagctatgatgagtgttgatgagacacttatgtgttcttttcaaattttgaagcctgcagacaagaagccaaagttctcatatacatccgtgagctcggcgaaaccaggaacgcctcctcgcggtgtgaagggcggaaagatttag

SEQ ID NO: 100 - DNA, 975 bp, Dennstaedtia punctilobula
atggacccagctgctttggaagacatcatccaccgtctcttggaagtgcgggagttgcggcctggcaagcaggtacagctctcagagggtgagatccggcagctctgctcgacatccaaggccatttttatgcaacagcccaacctgctggaactcgaagcgcccatcaagatctgcggtgacattcatggccagtattcagatctgttaaggctcttcgaatatggtggacttcctccacaggctaattatctctttttaggagattatgtggaccggggcaagcaaagtttggagaccatctgtcttcttctggcctataaaatcaagtacccagaaaacttttttctactgagaggcaatcacgaatgtgcttctattaatcgcatatatgggttttatgatgaatgcaagcggaggttcaatgtgaggttgtggaaggtctttactgactgtttcaattgtcttcctgtggctgctctgattgatgagaagattctttgtatgcatggcggactttcaccagaattgaacaacttggaccaaatcagagcattacctcggccaactgatgtgcctgatcaagggttactttgtgacctcctatgggcagatcccgagagggagatctcagggtggggtgagaatgatagaggcgtttctttcacatttggagcagacaaagttgatgaatttctacaaaaacatgatctcgaccttgtctgccgagctcatcaggttgtagaggatggctatgagtttttcgctgacagacagctcgtgaccatcttttcagctccaaattactgcggggaatttgacaattcaggagctatgatgagcgttgatgagacactgatgtgctcttttcagatcctcaagcctgctgagaagaagccgaagttttcactcacgtctgtcaatgcagcaaaaccagggacaccacctcgaggagcaaagggtggcaaagcttag

SEQ ID NO: 101 - DNA, 957 bp, Dennstaedtia punctilobula
atggatcctgctcttctagaaaatatcatagcaaggcttctaaatgtgcagtcggggcggcccggaaaacttgtccagatttcagaagctgagatcagacagctctgcctgcattccaaggacatctttctgcagcagcccaacttgttggagctcgaagcgccgatcaaaatctgtggtgacattcatggtcaatattcagatcttctcaggcttctcgaatacgggggatacccaccgcttgccaattacttgtttctaggggattatgtcgatcgtgggaagcagagcctagagactatatgtctcctgcttgcatataaaatcaagtacccagagaactttttcttgctcagaggcaaccatgagtgtgcttcgataaatcggatatatggcttctatgatgaatgtaagaggcgattcaatgtccgcatttggaagacgtttactgagtgtttcaactgcttgccagtggctgccctgattgatgagaagattctttgtatgcatggaggactttctccagacttgaaaaatttagatcaagtgcggaatattggccgcccgacagatgtcccagaggcagggcttttatgtgatttgctttggtcagatcctgaaagggatattcttggatggggggacaatgatagaggggtttcatacacttttggaacagacaaagttgttgaatttttgcgggcacatgatctggacctgatttgtcgggctcatcaggtggtggaagaaggttatgagttctttgcagagagagcattggtcaccatcttctctgctcctaattattgcggggagtttgacaatgcgggtgcgatgatgagtgtggacgagtcccttatgtgctctttccaaattctaaagcctacagaaaagaaaaacaaacttggctatgggagcgtgagcagaaccccgcgtccgaagggaggcaagacctag

SEQ ID NO: 102 - DNA, 957 bp, Dennstaedtia punctilobula
atggatcctgctcttctagaaaatatcatagcaaggcttctaaatgtgcagtcggggcggcccggaaaacttgtccagatttcagaagctgagatcagacagctctgcctgcattccaaggacatctttctgcagcagcccaacttgttggagctcgaagcgccaatcaaaatctgtggtgacattcatggtcaatattcagatcttctcaggcttctcgaatacgggggatacccaccgcttgccaattacttgtttctaggggattatgtcgatcgtgggaagcagagcctagagactatatgtctcctgcttgcatataaaatcaagtacccagagaactttttcttgctcagaggcaaccatgagtgtgcttcgataaatcggatatatggcttctatgatgaatgtaagaggcgattcaatgtccgcatttggaagacgtttactgagtgtttcaactgcttgccagtggctgccctgattgatgagaagattctttgtatgcatggaggactttctccagacttgaaaaatttagatcaagtgcggaatattggccgcccgacagatgtcccagaggcagggcttttatgtgatttgctttggtcagatcctgaaagggatattcttggatggggggacaatgatagaggggtttcatacacttttggaacagacaaagttgttgaatttttgcgggcacatgatctggacctgatttgtcgggctcatcaggtggtggaagaaggttatgagttctttgcagagagagcattggtcaccatcttctctgctcctaattattgcggggagtttgacaatgcgggtgcgatgatgagtgtggacgagtcccttatgtgctctttccaaattctaaagcctacagaaaagaaaaacaaacttggctatgggagcgtgagcagaaccccgcgtccgaagggaggcaagacctag

SEQ ID NO: 103 - DNA, 936 bp, Dennstaedtia punctilobula
atggaccctgctgcgctggaggatatcatacatcgtcttttggaagtgcgggatgccaggcctggcaagcaggtgcagctctcggaggctgagatccgacaactctgtctcacctctaaggctatcttcatgcaacagcccaacctgctcgaactagaagctcccatcaagatctgtggtgacattcgtggccagtattcagatctgttgcggctttttgaatacggtggactaccgccacaggcaaattatctttttttaggagattacgtggacaggggcaagcaaagtcttgaaaccatatgtctgttactagcctacaagatcaaatatcccgagaacttttttcttctgagaggaaatcatgaatgtgcgtctatcaatcgcatatatgggttttatgatgaatgcaagaggaggttcaacatcaggttgtggaagaccttcacggactgtttcaactgtctccctgtggcagctttgatagatgagaagattctttgcatgcatggtggcctttccccagaattgcacaaccttgaccaaatccgatcaatacctcgccccacagatgtacctgagaaagagctgtctgggtggggcgagaatgatagaggcgtatcttttacttttggtgcagataaagttagtgagtttttacagaagcatgatctcgacttgatctgccgagcccatcaggttgtagaggatggatatgaattttttgctgacagacagcttgtgacaattttttcagccccaaattactgtggggagtttgataatgcaggagctatgatgagtgttgatgagacacttatgtgttcttttcaaattttgaagcctgcagacaagaagccaaagttctcatatacatccgtgagctcggcgaaaccaggaacgcctcctcgcggtgtgaagggcggaaagatttag

SEQ ID NO: 104 - protein, 322 aa, Pennisetum glaucum
MAAATAAGGQGGGGMDNALLDDIIRRLLEVRTARPGKQVQLSEAEIRQLCTASREIFLSQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLTHLDEIKNLQRPTDVPDQGLLCDLLWSDPGKDVQGWGMNDRGVSYTFGADKVSEFLEKHDLDLICRAHQVVEDGYEFFADRQLVTIFSAPNYCGEFDNAGAMMSVDETLMCSFQILKPAERKNKFMGSNKM

SEQ ID NO: 105 - protein, 327 aa, Dennstaedtia punctilobula
MDPAVVDDIIARLLEVRSSRPGKQVQLSENEIRQLCSTSKTIFLQQPNLLELEAPIKICGDIHGQYSDLLRLFEYGGLPPQANYLFLGDYVDRGKQSLETICLLLAYKIKYPENFFLLRGNHECASINRIYGFYDECKRRFNVRLWKAFTECFNCLPVAALIDEKILCMHGGLSPELSNLDQIKNIYRPTDVPDQGLLCDLLWSDPDKEVSGWGENDRGVSYTFGVDKVIEFLEKHDLDLVCRAHQVVEEGYEFFADRRLVTIFSAPNYCGEFDNAGAMMSVDESLMCSFQILKPADKKPKFNYNNSSLARAGTPPRGVKPFQGGKV

SEQ ID NO: 106 - protein, 324 aa, Dennstaedtia punctilobula
MDPAALEDIIHRLLEVRDARPGKQVQLSEAEIRQLCLTSKAIFMQQPNLLELEAPIKICGDIHGQYSDLLRLFEYGGLPPQANYLFLGDYVDRGKQSLETICLLLAYEIKYPENFFLLRGNHECASINRIYGFYDECKRRFNIRLWKTFTDCFNCLPVAALIDEKILCMHGGLSPELHNLDQIRSIPRPTDVPDQGLLCDLLWADPEKELSGWGENDRGVSFTFGADKVSEFLQKHDLDLICRAHQVVEDGYEFFADRQLVTIFSAPNYCGEFDNAGAMMSVDETLMCSFQILKPADKKPKFSYTSVSSAKPGTPPRGVKGGKI

SEQ ID NO: 107 - protein, 324 aa, Dennstaedtia punctilobula
MDPAALEDIIHRLLEVRELRPGKQVQLSEGEIRQLCSTSKAIFMQQPNLLELEAPIKICGDIHGQYSDLLRLFEYGGLPPQANYLFLGDYVDRGKQSLETICLLLAYKIKYPENFFLLRGNHECASINRIYGFYDECKRRFNVRLWKVFTDCFNCLPVAALIDEKILCMHGGLSPELNNLDQIRALPRPTDVPDQGLLCDLLWADPEREISGWGENDRGVSFTFGADKVDEFLQKHDLDLVCRAHQVVEDGYEFFADRQLVTIFSAPNYCGEFDNSGAMMSVDETLMCSFQILKPAEKKPKFSLTSVNAAKPGTPPRGAKGGKA

SEQ ID NO: 108 - protein, 318 aa, Dennstaedtia punctilobula
MDPALLENIIARLLNVQSGRPGKLVQISEAEIRQLCLHSKDIFLQQPNLLELEAPIKICGDIHGQYSDLLRLLEYGGYPPLANYLFLGDYVDRGKQSLETICLLLAYKIKYPENFFLLRGNHECASINRIYGFYDECKRRFNVRIWKTFTECFNCLPVAALIDEKILCMHGGLSPDLKNLDQVRNIGRPTDVPEAGLLCDLLWSDPERDILGWGDNDRGVSYTFGTDKVVEFLRAHDLDLICRAHQVVEEGYEFFAERALVTIFSAPNYCGEFDNAGAMMSVDESLMCSFQILKPTEKKNKLGYGSVSRTPRPKGGKT

SEQ ID NO: 109 - protein, 318 aa, Dennstaedtia punctilobula; identical to SEQ ID NO: 108

SEQ ID NO: 110 - protein, 311 aa, Dennstaedtia punctilobula
MDPAALEDIIHRLLEVRDARPGKQVQLSEAEIRQLCLTSKAIFMQQPNLLELEAPIKICGDIRGQYSDLLRLFEYGGLPPQANYLFLGDYVDRGKQSLETICLLLAYKIKYPENFFLLRGNHECASINRIYGFYDECKRRFNIRLWKTFTDCFNCLPVAALIDEKILCMHGGLSPELHNLDQIRSIPRPTDVPEKELSGWGENDRGVSFTFGADKVSEFLQKHDLDLICRAHQVVEDGYEFFADRQLVTIFSAPNYCGEFDNAGAMMSVDETLMCSFQILKPADKKPKFSYTSVSSAKPGTPPRGVKGGKI

SEQ ID NO: 111 - protein, 323 aa, Paspalum notatum
MSAAPAAGGQGGGGGMDPALLDDIIRRLLEVRTARPGKQVQLSESEIRQLCTVSREIFLSQPNLLELEAPIKICGDIHGQYSDLLRLFEYGGFPPEANYLFLGDYVDRGKQSLETICLLLAYKIKYPENFFLLRGNHECASINRIYGFYDECKRRFNVRLWKVFTECFNTLPVAALIDDKILCMHGGLSPDLGHLDEIKNLQRPTDVPDQGLLCDLLWSDPGKDVQGWGMNDRGVSYTVGPDKVSEFLQKHDLDLICRAHQVVEDGYEFFADRQLVTIFSAPNYRGEFDNAGAMMSVDETLMCSFQILKPAERKGKFMSSNKM

SEQ ID NO: 112 - DNA, 972 bp, Paspalum notatum
atgtcggcggcgccggcggcgggagggcaggggggaggcgggggcatggaccccgcgctactcgacgacatcatccgccgcctgctcgaggtgcggaccgcgcggcctgggaagcaggtgcagctctccgagtcggagatccgccagctctgcaccgtctcccgcgaaatcttcctcagccagcccaatctcctcgagctcgaggcgcctatcaagatctgcggtgacatccatggtcaatatagtgatctgttaaggctttttgaatatggaggcttcccacctgaggcaaattatctattcttaggtgattatgttgatcgaggcaaacaaagtttggagactatatgccttcttcttgcatacaaaatcaagtacccagaaaacttctttcttctgaggggcaatcatgaatgtgcctcaataaatagaatatatggattttatgatgaatgcaagcgtcgatttaatgtgcggctatggaaggtcttcactgaatgttttaacacactcccagtggctgctctaatagatgataaaatattatgtatgcacggtggactctctcctgatctaggacacttggatgagataaagaacttgcaacgtccaactgatgtacctgatcaaggcctactatgcgacttgctttggtcagatccaggaaaagacgttcaagggtggggcatgaatgatagaggggtctcatataccgttggtcctgacaaagtttcagaattcttgcaaaaacatgatcttgatcttatttgtcgggctcaccaggtggttgaggatggatacgaattctttgctgacagacagctggttaccatattctcagcccccaattatcgtggtgaatttgataatgctggtgcgatgatgagcgtcgatgaaactttgatgtgttcattccaaattctcaaacctgctgagagaaaaggcaaatttatgagttcaaacaaaatgtga

SEQ ID NO: 113 - protein, 318 aa, Arabidopsis thaliana; identical to SEQ ID NO: 39

SEQ ID NO: 114 - DNA, 957 bp, Arabidopsis thaliana
atgatgacgagtatggaagggatggtggagaaaggagtattggatgatattataagaagattgttagaaggtaaaggaggcaaacaggttcagctttccgagagcgagattcgtcaactctgctttaacgctcgtcaaatcttcctctctcaacctaatctccttgatctccatgccccaattcgcatctgcggtgatattcatggtcaatatcaagatcttttgaggttgtttgaatacggaggttatcctccttcagcaaactatctattccttggtgattacgttgacagaggcaaacaaagtcttgagaccatatgtttgcttcttgcttacaagatacggtacccatcgaagatatatctgttgagagggaaccatgaggatgctaagatcaacaggatttacgggttttatgacgagtgcaaacggagattcaatgtacgactctggaaggtgtttactgattgcttcaactgtttacctgtagctgcacttattgatgagaagatactgtgtatgcacggtggtttgtcaccagatttggataatttgaatcagattcgagagattcaaaggcctattgagattccagacagtggtcttctttgtgatttactttggtcagatcctgatcagaagattgaaggttgggctgatagtgatcgaggtatctcttgcacttttggagctgataaagtcgctgagttcttggataagaatgatcttgacctcatttgccgaggccatcaggtagtagaagacgggtatgagtttttcgcaaaacggagattagtcacgatattctcagctccaaactatggtggggagtttgacaacgctggtgcgttattaagcgttgacgagtctcttgtatgttcctttgagattatgaaaccagccccagcttcaagcagtcatcctctcaagaaggtaccgaagatggggaagtcttga

SEQ ID NO: 115 - protein, 327 aa, Arabidopsis thaliana
MDPGTLNSVINRLLEAREKPGKIVQLSETEIKQLCFVSRDIFLRQPNLLELEAPVKICGDIHGQYPDLLRLFEHGGYPPNSNYLFLGDYVDRGKQSLETICLLLAYKIKFPENFFLLRGNHESASINRIYGFYDECKRRFSVKIWRIFTDCFNCLPVAALIDERIFCMHGGLSPELLSLRQIRDIRRPTDIPDRGLLCDLLWSDPDKDVRGWGPNDRGVSYTFGSDIVSGFLKRLDLDLICRAHQVVEDGFEFFANKQLVTIFSAPNYCGEFDNAGAMMSVSEDLTCSFQILKSNDKKSKFSFGSRGGAKTSFPYPKVKVCINHITF

SEQ ID NO: 116 - DNA, 984 bp, Arabidopsis thaliana
atggatcctggtactttaaactcggtgatcaataggttgcttgaagctagagaaaaaccaggaaagattgttcagttgtctgaaacagagatcaaacagctctgtttcgtctctagagatatcttcttgagacaaccaaatctcttggaacttgaagctcctgttaaaatatgtggggacattcatggacaatatccggatctcttgagactattcgaacatggcggataccctcctaattcaaactacttgtttcttggagattatgtcgatcgcggcaagcaaagcctcgaaacgatttgtcttttacttgcttacaagattaagttccctgaaaacttcttccttctcagaggaaaccatgaaagtgcatcaatcaatcgtatttacggcttctatgacgagtgtaaacgtagattcagtgtcaagatttggcgaatcttcactgattgcttcaactgtctccccgtcgctgcactcatcgatgagcggattttttgtatgcatggtgggctctccccggagctgctaagcttgaggcagattagggatattcgtcgtccaacggatattcctgatcgtggtttactctgtgatctcttgtggtctgatcctgataaagatgttagaggttgggggcctaacgatcgcggagtttcttacacttttggatcagatatagtttctggatttcttaaaagactcgatcttgacctcatttgtagggctcaccaggttgttgaagatggattcgagttctttgcgaataagcagctcgtaacgatattctctgcgccgaattactgtggggaatttgacaatgcaggtgcgatgatgagtgtgtctgaggatttgacctgctcttttcagatcttaaaatctaatgacaagaaatcaaagttcagtttcggaagcagaggtggtgctaaaactagcttcccttatcctaaagtgaaggtatgtattaatcacatcactttttaa

SEQ ID NO: 117 - protein, 323 aa, Arabidopsis thaliana
MDPGTLNSVINRLLEAREKPGKIVQLSETEIKQLCFVSRDIFLRQPNLLELEAPVKICGDIHGQYPDLLRLFEHGGYPPNSNYLFLGDYVDRGKQSLETICLLLAYKIKFPENFFLLRGNHESASINRIYGFYDECKRRFSVKIWRIFTDCFNCLPVAALIDERIFCMHGGLSPELLSLRQIRDIRRPTDIPDRGLLCDLLWSDPDKDVRGWGPNDRGVSYTFGSDIVSGFLKRLDLDLICRAHQVVEDGFEFFANKQLVTIFSAPNYCGEFDNAGAMMSVSEDLTCSFQILKSNDKKSKFSFGSRGGAKTSFPYPKVKDCNW

SEQ ID NO: 118 - DNA, 972 bp, Arabidopsis thaliana
atggatcctggtactttaaactcggtgatcaataggttgcttgaagctagagaaaaaccaggaaagattgttcagttgtctgaaacagagatcaaacagctctgtttcgtctctagagatatcttcttgagacaaccaaatctcttggaacttgaagctcctgttaaaatatgtggggacattcatggacaatatccggatctcttgagactattcgaacatggcggataccctcctaattcaaactacttgtttcttggagattatgtcgatcgcggcaagcaaagcctcgaaacgatttgtcttttacttgcttacaagattaagttccctgaaaacttcttccttctcagaggaaaccatgaaagtgcatcaatcaatcgtatttacggcttctatgacgagtgtaaacgtagattcagtgtcaagatttggcgaatcttcactgattgcttcaactgtctccccgtcgctgcactcatcgatgagcggattttttgtatgcatggtgggctctccccggagctgctaagcttgaggcagattagggatattcgtcgtccaacggatattcctgatcgtggtttactctgtgatctcttgtggtctgatcctgataaagatgttagaggttgggggcctaacgatcgcggagtttcttacacttttggatcagatatagtttctggatttcttaaaagactcgatcttgacctcatttgtagggctcaccaggttgttgaagatggattcgagttctttgcgaataagcagctcgtaacgatattctctgcgccgaattactgtggggaatttgacaatgcaggtgcgatgatgagtgtgtctgaggatttgacctgctcttttcagatcttaaaatctaatgacaagaaatcaaagttcagtttcggaagcagaggtggtgctaaaactagcttcccttatcctaaagtgaaggattgtaattggtag

(19) United States
(12) Patent Application Publication
(10) Pub. No.: US 2014/0259225 A1
(43) Pub. Date: Sep. 11, 2014

(54) MANIPULATION OF SERINE/THREONINE PROTEIN PHOSPHATASES FOR CROP IMPROVEMENT

(71) Applicant: PIONEER HI BRED INTERNATIONAL INC, Johnston, IA (US)

(72) Inventors: Mary Frank, Des Moines, IA (US); Rajeev Gupta, Johnston, IA (US); Kristin Haug Collet, Des Moines, IA (US); Bo Shen, Johnston, IA (US); Carl R. Simmons, Des Moines, IA (US); Jingrui Wu, Johnston, IA (US); Wengang Zhou, Johnston, IA (US)

(73) Assignee: PIONEER HI BRED INTERNATIONAL INC, Johnston, IA (US)

(21) Appl. No.: 13/926,393

(22) Filed: Jun. 25, 2013

Related U.S. Application Data
(60) Provisional application No. 61/666,177, filed on Jun. 29, 2012.
(60) Provisional application No. 61/778,550, filed on Mar. 13, 2013.

(51) Int. Cl. C12N 15/82

(52) U.S. Cl. CPC C12N 15/8243
USPC 800/290; 800/278; 800/298; 435/6.11; 435/320.1; 800/320.1; 536/23.2

(57) ABSTRACT

Methods and compositions relating to altering nitrogen utilization and/or uptake or yield in plants. Recombinant expression cassettes, host cells and transgenic plants are described. Serine-threonine protein phosphatases improve agronomic traits of a crop plant.

### CROSS-REFERENCE

This utility application claims the benefit U.S. Provisional Application Ser. No. 61/778,550, filed Mar. 13, 2013, which is incorporated herein by reference.

### FIELD

The disclosure relates generally to the field of molecular biology, specifically the modulation of plant fertility to improve plant stress tolerance.

### BACKGROUND

The domestication of many plants has correlated with dramatic increases in yield. Most phenotypic variation occurring in natural populations is continuous and is affected by multiple gene influences. The identification of specific genes responsible for the dramatic differences in yield in domesticated plants has become an important focus of agricultural research.

The global demand for nitrogen (N) fertilizer for agricultural production, which already stands at ˜90 million metric tons per year, is projected to increase to 240 million metric tons by the year 2050. Because nitrate is very mobile in the soil, substantial amount of applied N is lost by leaching, run-off and de-nitrification. In addition to increase in cost of crop production, in the long run these processes of N loss not only pollute the ground water and adversely effects soil structure but also has detrimental effects on the environment such as increase in nitric oxide, ozone etc. Hence, developing crop varieties with improved efficiency for N absorption and utilization will help mitigate these problems to some extent. ‘Signaling’ affects almost all aspects of life and protein phosphorylation/dephosphorylation plays a major role in regulating ‘signaling’ and numerous other biological processes. Phosphorylation and dephosphorylation are catalyzed by protein kinases and phosphatases respectively, which account for ˜5% of Arabidopsis genome, suggesting a major role for them in life cycle of plants. Among protein phosphatases, serine-threonine protein phosphatase (STPP) is the major multi-gene family in higher plants including maize.

### SUMMARY

One embodiment relates to an isolated polynucleotide comprising a nucleotide sequence selected from the group consisting of: (a) the nucleotide sequence comprising SEQ ID NO: 48-94, 97-103, 112, 114, 116 and 118 (b) the nucleotide sequence encoding an amino acid sequence comprising SEQ ID NO: 1-47, 104-111, 113, 115 and 117 and (c) the nucleotide sequence comprising at least 70% sequence identity to SEQ ID NO: 48-94, 97-103, 112, 114, 116 and 118, wherein said polynucleotide encodes a polypeptide affecting NUE activity and/or yield.

Compositions include an isolated polypeptide comprising an amino acid sequence selected from the group consisting of: (a) the amino acid sequence comprising SEQ ID NO: 1-47, 104-111, 113, 115 and 117 and (b) the amino acid sequence comprising at least 70% sequence identity to SEQ ID NO: 1-47, 104-111, 113, 115 and 117 wherein said polypeptide has effects on NUE and/or yield.

Modulation of expression of STPP in a plant can improve the nitrogen stress tolerance of the plant and such plants can maintain their productive rates with significantly less nitrogen fertilizer input and/or exhibit enhanced uptake and assimilation of nitrogen fertilizer and/or remobilization and reutilization of accumulated nitrogen reserves. In addition to an overall increase in yield, the improvement of nitrogen stress tolerance through expression of STPP can also result in increased root mass and/or length, increased ear, leaf, seed and/or endosperm size, and/or improved standability. Accordingly, in some embodiments, the methods further comprise growing said plants under nitrogen limiting conditions and optionally selecting those plants exhibiting greater tolerance to the low nitrogen levels.

Further, methods and compositions are provided for improving yield under abiotic stress, which include evaluating the environmental conditions of an area of cultivation for abiotic stressors (e.g., low nitrogen levels in the soil) and planting seeds or plants having reduced male fertility, in stressful environments.

Constructs and expression cassettes comprising nucleotide sequences that can efficiently modify expression of STPP are also provided herein.

Recombinant expression cassettes comprising a nucleic acid disclosed herein are described. Vectors containing the recombinant expression cassettes can facilitate the transcription and translation of the nucleic acid in a host cell. Host cells able to express the polynucleotides are described. A number of host cells could be used, such as but not limited to, microbial, plant or insect.

Plants containing the polynucleotides disclosed herein include but are not limited to maize, soybean, sunflower, sorghum, canola, wheat, alfalfa, cotton, rice, barley, tomato and millet. In another embodiment, the transgenic plant is a maize plant or plant cells. Another embodiment is the transgenic seeds from the transgenic serine/threonine protein phosphatase polypeptide of the disclosure operably linked to a promoter that drives expression in the plant. The plants of the disclosure can have altered NUE as compared to a control plant. In some plants, the NUE is altered in a vegetative tissue, a reproductive tissue or a vegetative tissue and a reproductive tissue. Plants can have at least one of the following phenotypes including but not limited to: increased root mass, increased root length, increased leaf size, increased ear size, increased seed size, increased green color, increased endosperm size.

Plants that have been genetically modified at a genomic locus, wherein the genomic locus encodes a type I serine/threonine protein phosphatase disclosed herein, for example a recombinant regulatory element increasing the expression of an endogenous serine threonine protein phosphatase.

Methods for increasing the activity of a serine/threonine protein phosphatase in a plant are provided. The method can comprise introducing into the plant a serine/threonine protein phosphatase polynucleotides.

A method of increasing yield or an agronomic parameter that contributes to yield, the method includes increasing the expression or activity of a serine threonine protein phosphatase (STPP) in a plant; and growing the plant in a plant growing environment.

In an embodiment, the serine threonine protein phosphatase is of type 1. In an embodiment, the STPP is maize STPP3.

A method of improving an agronomic characteristic of a plant, the method includes increasing the expression or activity of a serine threonine protein phosphatase (STPP) in a plant, wherein the STPP polypeptide comprises a metallophos domain (PFAM PF00149.22); and improving the agronomic characteristic of the plant by growing the plant in a plant growing environment.

In an embodiment, the STPP polypeptide comprises a motif near the N-terminus comprising an amino acid sequence of L[L/T]EVR[T/L]ARPGKQVQL and a motif near the C-terminus comprising an amino acid sequence of GAMMSVDE[T/N]LMCSFQ.

In an embodiment, the STPP polypeptide comprises the amino acid sequence of VRTARPGKQV.

In an embodiment, the STPP polypeptide comprises the amino acid sequence of selected from the group comprising SEQ ID NO: 1-47, 104-111, 113, 115 or 117, or a variant that is at least 90% similar to SEQ ID NO: 1-47, 104-111, 113, 115 or 117.

A plant includes in its genome a recombinant serine threonine protein phosphatase (STPP), wherein the protein phosphatase includes a motif near the N-terminus comprising an amino acid sequence of L[L/T]EVR[T/L]ARPGKQVQL (SEQ ID NO: 95), a motif near the C-terminus comprising an amino acid sequence of GAMMSVDE[T/N]LMCSFQ (SEQ ID NO: 96), an RVxF binding site, a catalytic subunit and a regulatory subunit and wherein the plant exhibits an improved agronomic characteristic. In an embodiment, the plant exhibits an increase in nitrogen use efficiency as compared to a control plant that does not include a recombinant STPP in it genome.

A plant includes in its genome a heterologous regulatory element operably linked to a serine threonine protein phosphatase (STPP), wherein the heterologous regulatory element increases the expression of the protein phosphatase, the protein phosphatase comprises a motif near the N-terminus comprising an amino acid sequence of L[L/T]EVR[T/L]ARPGKQVQL (SEQ ID NO: 95), a motif near the C-terminus comprising an amino acid sequence of GAMMSVDE[T/N]LMCSFQ (SEQ ID NO: 96), an RVxF binding site, a catalytic subunit and a regulatory subunit and wherein the plant exhibits an improved agronomic characteristic. In an embodiment, the heterologous regulatory element is an enhancer. In an embodiment, the heterologous regulatory element is a promoter.

A method of identifying and selecting an allele of ZmSTPP3, the allele results in an increased expression of the ZmSTPP3 polypeptide and/or an increased enzymatic activity, the method includes performing a genetic screen on a population of mutant maize plants; identifying one or more mutant maize plants that exhibit the increased expression of the ZmSTPP3 polypeptide and/or the increased enzymatic activity; and identifying the ZmSTPP3 allele from the mutant maize plant. In an embodiment, the maize mutant plant is sequenced at a locus comprising ZmSTPP3.

A method of increasing nitrogen uptake in a plant, the method includes increasing the expression or activity of a serine threonine protein phosphatase (STPP) in a plant, wherein the STPP polypeptide comprises a metallophos domain (PFAM PF00149); and improving the nitrogen uptake of the plant by growing the plant in a plant growing environment.

In an embodiment, the STPP polypeptide comprises the amino acid sequence of VRTARPGKQV.

A recombinant DNA construct capable of being expressed in a plant cell, the construct includes a polynucleotide expressing a serine threonine protein phosphatase (STPP) in a plant, wherein the STPP polypeptide comprises a metallophos domain (PFAM PF00149); heterologous promoter operably linked to the protein phosphatase and functional in plant cells; and a transcriptional terminator functional in plant cells.

A maize plant includes the DNA constructs described herein. In an embodiment, the DNA constructs encode a STPP that includes a polynucleotide sequence that encodes the protein phosphatase comprising a sequence that is at least 80% similar to one selected from the group comprising SEQ ID NO: 48-94, 97-103, 112, 114, 116 and 118.

A method of improving nitrogen utilization efficiency of a monocot plant, the method includes increasing the expression or activity of a serine threonine protein phosphatase (STPP) in a plant, wherein the STPP polypeptide comprises a metallophos domain (PFAM PF00149) and further comprises a motif near the N-terminus comprising an amino acid sequence of L[L/T]EVR[T/L]ARPGKQVQL (SEQ ID NO: 95) or a motif near the C-terminus comprising an amino acid sequence of GAMMSVDE[T/N]LMCSFQ (SEQ ID NO: 96); and growing the plant in a plant growing condition, wherein the rate of application of a nitrogen fertilizer is less than about 140 to 160 pounds/acre.

A method of increasing field yield of a monocot plant by improving nitrogen utilization efficiency of a monocot plant, the method includes increasing the expression or activity of a serine threonine protein phosphatase (STPP) in a plant, wherein the STPP polypeptide comprises a metallophos domain (PFAM PF00149) and further comprises a motif near the N-terminus comprising an amino acid sequence of L[L/T]EVR[T/L]ARPGKQVQL (SEQ ID NO: 95) or a motif near the C-terminus comprising an amino acid sequence of GAMMSVDE[T/N]LMCSFQ (SEQ ID NO: 96); and growing the plant in a plant growing condition, wherein the rate of application of a nitrogen fertilizer is about 140 to 160 pounds/acre.

A plant includes in its genome a recombinant DNA construct comprising an isolated polynucleotide operably linked, to a promoter functional in a plant, wherein the polynucleotide includes (a) the nucleotide sequence of selected from the group comprising SEQ ID NO: 48-94, 97-103, 112, 114, 116 and 118; (b) a nucleotide sequence with at least 90% sequence identity, based on the Clustal V method of alignment, when compared to one selected from the group comprising SEQ ID NO: 48-94, 97-103, 112, 114, 116 and 118 or (c) a nucleotide sequence that can hybridize under stringent conditions with the nucleotide sequence of (a) and wherein the plant exhibits an alteration in at least one agronomic characteristic selected from the group consisting of: enlarged ear meristem, kernel row number, seed number, plant height, biomass and yield, when compared to a control plant not comprising the recombinant DNA construct.

In an embodiment, a plant is selected from the group consisting of: Arabidopsis, tomato, maize, soybean, sunflower, sorghum, canola, wheat, alfalfa, cotton, rice, barley, millet, sugar cane and switchgrass.

Seeds of the plants described herein exhibit an alteration in at least one agronomic characteristic selected from the group consisting of: enlarged ear meristem, kernel row number, seed number, plant height, biomass and yield, when compared to a control plant not comprising the recombinant DNA construct.

A recombinant polynucleotide that encodes a serine threonine protein phosphatase (STPP) in a plant, wherein the STPP polypeptide includes a metallophos domain (PFAM PF00149.22) and further includes a motif near the N-terminus comprising an amino acid sequence of L[L/T]EVR[T/L]ARPGKQVQL and a motif near the C-terminus comprising an amino acid sequence of GAMMSVDE[T/N]LMCSFQ.

A method of improving yield of a maize plant, the method includes providing a maize plant that has in its genome a recombinant polynucleotide encoding a polypeptide that is at least 90% identical to SEQ ID NO: 1 and increasing grain yield of the maize plant by growing the maize plant in a plant growing environment. In an embodiment, the transgenic maize plant includes in its genome a recombinant polynucleotide encoding a polypeptide that is at least 90% identical to SEQ ID NO: 1.

A method of improving yield of a maize plant, the method includes providing a maize plant that contains in its genome a recombinant polynucleotide encoding a polypeptide that is at least 90% identical to a sequence selected from the group consisting of SEQ ID NOS: 1-8 and increasing grain yield of the maize plant by growing the maize plant in a plant growing environment.

A transgenic maize plant includes in its genome a recombinant polynucleotide encoding a polypeptide that is at least 90% identical to a sequence selected from the group consisting of SEQ ID NOS: 1-8. A transgenic monocot crop plant includes in its genome a recombinant polynucleotide encoding a polypeptide that is at least 90% identical to a sequence selected from the group consisting of SEQ ID NOS: 1-8.

A method of improving yield of a maize plant, the method comprising providing a maize plant comprising in its genome a recombinant polynucleotide encoding a polypeptide that is at least 85% identical to SEQ ID NO: 1 and increasing grain yield of the maize plant by growing the maize plant in a plant growing environment. In an embodiment, the polypeptide is about 87% identical to SEQ ID NO: 1.

A transgenic maize plant includes in its genome a recombinant polynucleotide encoding a polypeptide that is at least 85% identical to SEQ ID NO: 1. In an embodiment, the maize plant include a polypeptide that is about 87% identical to SEQ ID NO: 1. In an embodiment, the transgenic maize plant yields at least about 3-5 bu/acre more compared to a control plant not containing the recombinant polynucleotide.

Methods for reducing or eliminating the level of a serine/threonine protein phosphatase polypeptide in the plant are provided. The level or activity of the polypeptide could also be reduced or eliminated in specific tissues, causing alteration in plant growth rate. Reducing the level and/or activity of the serine/threonine protein phosphatase polypeptide may lead to smaller stature or slower growth of plants.

### BRIEF DESCRIPTION OF THE DRAWINGS

FIG. 1 (FIG. 1A-1I) shows alignment of the STPP sequences with conserved motifs identified. Two motifs are designated (SEQ ID NO: 95 and 96).

FIG. 2 shows a dendrogram containing the relationship of the STPP sequences and their identification into clades. The cluster designations of Table 1 correspond to key branch points within FIG. 2. The evolutionary history was inferred by using the Maximum Likelihood method based on the JTT matrix-based model. The tree with the highest log likelihood (−5257.1242) is shown. Initial tree(s) for the heuristic search were obtained automatically as follows. When the number of common sites was <100 or less than one fourth of the total number of sites, the maximum parsimony method was used; otherwise BIONJ method with MCL distance matrix was used. The tree is drawn to scale, with branch lengths measured in the number of substitutions per site. The analysis involved 55 amino acid sequences. All positions containing gaps and missing data were eliminated. There were a total of 273 positions in the final dataset. Evolutionary analyses were conducted in MEGA5.

FIG. 3 demonstrates multi-events/years/testers/locations yield data analyses of transgenic over-expressing ZmSTPP3 tested under low and normal N conditions. BLUP analyses of events in low N (bottom panel), normal N (middle panel) and low N/normal N combined (top panel) showed an increase of 2-5 bu/acre. Blue bars represent events with statistically significant differences. The data from 81 replications are presented in this Figure.

FIG. 4 represents data from two transgenic fast cycling corn events of ZmSTPP3 to demonstrate improved ear traits in NUE reproductive assay. Values plotted are % increase of transgenic events over controls. * indicates P<0.1.

### DETAILED DESCRIPTION

ZmSTPP3 shows increased maize grain yield under normal and low nitrogen conditions in multiple year trials. Maize lines overexpressing STPP3 had significantly higher nitrogen use efficiency than controls.

Nitrogen utilization efficiency (NUE) genes affect yield and have utility for improving the use of nitrogen in crop plants, especially maize. Increased nitrogen use efficiency can result from enhanced uptake and assimilation of nitrogen fertilizer and/or the subsequent remobilization and reutilization of accumulated nitrogen reserves, as well as increased tolerance of plants to stress situations such as low nitrogen environments. The genes can be used to alter the genetic composition of the plants, rendering them more productive with current fertilizer application standards or maintaining their productive rates with significantly reduced fertilizer or reduced nitrogen availability. Improving NUE in corn would increase corn harvestable yield per unit of input nitrogen fertilizer, both in developing nations where access to nitrogen fertilizer is limited and in developed nations where the level of nitrogen use remains high. Nitrogen utilization improvement also allows decreases in on-farm input costs, decreased use and dependence on the non-renewable energy sources required for nitrogen fertilizer production and reduces the environmental impact of nitrogen fertilizer manufacturing and agricultural use.

Methods and compositions for improving plant yield are provided. In some embodiments, plant yield is improved under stress, particularly abiotic stress, such as nitrogen limiting conditions.

Polynucleotides, related polypeptides and all conservatively modified variants of STPP genes involved in nitrogen metabolism in plants are disclosed.

Methods to alter the genetic composition of crop plants, especially maize, so that such crops can be more productive with current fertilizer applications and/or as productive with significantly reduced fertilizer input are disclosed. Yield enhancement and reduced fertilizer costs with corresponding reduced impact to the environment are disclosed.

The STPP molecules described are comprised of a 2 subunits: the first being a catalytic subunit which is highly conserved and ubiquitous; and a second regulatory subunit which defines diverse functions and specificity. The regulatory subunit targets proteins to cellular locations and modulates their activities. The serine/threonine protein phosphatases were initially categorized into two groups, PP1 and PP2 (PP2A, PP2B, PP2C), based on their substrate specificity and pharmacological properties. PP1 is a ubiquitous and highly conserved enzyme found in all eukaryotes. Mammalian PP1 involved in regulation of glycogen biosynthesis, cell cycle, and muscle contraction. Function of plant PP1 was not known. PP2A regulates the activities of key enzymes, such as nitrate reductase and sucrose phosphate synthase, hormone signaling and defense signaling.

All references referred to are incorporated herein by reference.

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide or polypeptide where the additional sequences do not materially affect the basic function of the claimed polynucleotide or polypeptide sequences.

The term “construct” is used to refer generally to an artificial combination of polynucleotide sequences, i.e. a combination which does not occur in nature, normally comprising one or more regulatory elements and one or more coding sequences. The term may include reference to expression cassettes and/or vector sequences, as is appropriate for the context.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control plant may also be a plant transformed with an alternative down-regulation construct.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The terms “non-naturally occurring”; “mutated”, “recombinant”; “recombinantly expressed”; “heterologous” or “heterologously expressed” are representative biological materials that are not present in its naturally occurring environment.

The term “NUE nucleic acid” means a nucleic acid comprising a polynucleotide (“NUE polynucleotide”) encoding a full length or partial length polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA corresponding to the second sequence. Generally, operably linked means that the nucleic acid sequences being linked are contiguous and, where necessary, to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example) and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter which is active in essentially all tissues of a plant, under most environmental conditions and states of development or cell differentiation.

The term “polypeptide” refers to one or more amino acid sequences. The term is also inclusive of fragments, variants, homologs, alleles or precursors (e.g., preproproteins or proproteins) thereof. A “NUE protein” comprises a polypeptide. Unless otherwise stated, the term “NUE nucleic acid” means a nucleic acid comprising a polynucleotide (“NUE polynucleotide”) encoding a polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

TABLE 1

| SEQ ID | POLYNUCLEOTIDE/ | | | |
|---|---|---|---|---|
| NUMBER | POLYPEPTIDE | SPECIES | Short Name | Cluster Designation |
| SEQ ID | Polypeptide | Zea mays | ZM-STPP3 | Cluster 1.1/Cluster 1 |
| NO: 1 | | | | |
| SEQ ID | Polypeptide | Zea mays | ZM-STPP3-2 | Cluster 1.1/Cluster 1 |
| NO: 2 | | | | |
| SEQ ID | Polypeptide | Zea mays | ZM-STPP3-1 | Cluster 1.1/Cluster 1 |
| NO: 3 | | | | |
| SEQ ID | Polypeptide | Sorghum | SB-STPP3-1 | Cluster 1.1/Cluster 1 |
| NO: 4 | | bicolor | | |
| SEQ ID | Polypeptide | Oryza sativa | OS-STPP3-1 | Cluster 1.1/Cluster 1 |
| NO: 5 | | | | |
| SEQ ID | Polypeptide | Arabidopsis | AT-TOPP4 | Cluster 1.1/Cluster 1 |
| NO: 6 | | thaliana | | |
| SEQ ID | Polypeptide | Glycine max | GM-STPP-1 | Cluster 1.1/Cluster 1 |
| NO: 7 | | | | |
| SEQ ID | Polypeptide | Glycine max | GM-STPP-2 | Cluster 1.1/Cluster 1 |
| NO: 8 | | | | |
| SEQ ID | Polypeptide | Glycine max | GM-STPP-3 | Cluster 1.2/Cluster 1 |
| NO: 9 | | | | |
| SEQ ID | Polypeptide | Glycine max | GM-STPP-4 | Cluster 1.2/Cluster 1 |
| NO: 10 | | | | |
| SEQ ID | Polypeptide | Glycine max | GM-STPP-5 | Cluster 1.2/Cluster 1 |
| NO: 11 | | | | |
| SEQ ID | Polypeptide | Glycine max | GM-STPP-6 | Cluster 1.2/Cluster 1 |
| NO: 12 | | | | |
| SEQ ID | Polypeptide | Oryza sativa | OS-STPP3-2 | Cluster 1.2/Cluster 1 |
| NO: 13 | | | | |
| SEQ ID | Polypeptide | Sorghum | SB-STPP3-2 | Cluster 1.2/Cluster 1 |
| NO: 14 | | bicolor | | |
| SEQ ID | Polypeptide | Zea mays | ZM-STPP3-3 | Cluster 1.2/Cluster 1 |
| NO: 15 | | | | |
| SEQ ID | Polypeptide | Zea mays | ZM-STPP3-4 | Cluster 1.2/Cluster 1 |
| NO: 16 | | | | |
| SEQ ID | Polypeptide | Arabidopsis | AT-TOPP1 | Cluster 1.3/Cluster 1 |
| NO: 17 | | thaliana | | |
| SEQ ID | Polypeptide | Arabidopsis | AT-TOPP5 | Cluster 1.3/Cluster 1 |
| NO: 18 | | thaliana | | |
| SEQ ID | Polypeptide | Arabidopsis | AT-TOPP2 | Cluster 1.3/Cluster 1 |
| NO: 19 | | thaliana | | |
| SEQ ID | Polypeptide | Arabidopsis | AT-TOPP7-1 | Cluster 1.4/Cluster 1 |
| NO: 20 | | thaliana | | |
| SEQ ID | Polypeptide | Glycine max | GM-STPP-7 | Cluster 1.4/Cluster 1 |
| NO: 21 | | | | |
| SEQ ID | Polypeptide | Glycine max | GM-STPP-8 | Cluster 1.4/Cluster 1 |
| NO: 22 | | | | |
| SEQ ID | Polypeptide | Glycine max | GM-STPP-9 | Cluster 1.4/Cluster 1 |
| NO: 23 | | | | |
| SEQ ID | Polypeptide | Glycine max | GM-STPP-10 | Cluster 1.4/Cluster 1 |
| NO: 24 | | | | |
| SEQ ID | Polypeptide | Glycine max | GM-STPP-11 | Cluster 2.1/Cluster 2 |
| NO: 25 | | | | |
| SEQ ID | Polypeptide | Glycine max | GM-STPP-12 | Cluster 2.1/Cluster 2 |
| NO: 26 | | | | |
| SEQ ID | Polypeptide | Arabidopsis | AT-TOPP6 | Cluster 2.1/Cluster 2 |
| NO: 27 | | thaliana | | |
| SEQ ID | Polypeptide | Arabidopsis | AT-TOPP3 | Cluster 2.1/Cluster 2 |
| NO: 28 | | thaliana | | |
| SEQ ID | Polypeptide | Zea mays | ZM-STPP3-8 | Cluster 2.2/Cluster 2 |
| NO: 29 | | | | |
| SEQ ID | Polypeptide | Sorghum | SB-STPP3-4 | Cluster 2.2/Cluster 2 |
| NO: 30 | | bicolor | | |
| SEQ ID | Polypeptide | Oryza sativa | OS-STPP3-4 | Cluster 2.2/Cluster 2 |
| NO: 31 | | | | |
| SEQ ID | Polypeptide | Glycine max | GM-STPP-13 | Cluster 2.2/Cluster 2 |
| NO: 32 | | | | |
| SEQ ID | Polypeptide | Oryza sativa | OS-STPP3-3 | Cluster 2.3/Cluster 2 |
| NO: 33 | | | | |
| SEQ ID | Polypeptide | Zea mays | ZM-STPP3-5 | Cluster 2.3/Cluster 2 |
| NO: 34 | | | | |
| SEQ ID | Polypeptide | Sorghum | SB-STPP3-3 | Cluster 2.3/Cluster 2 |
| NO: 35 | | bicolor | | |
| SEQ ID | Polypeptide | Zea mays | ZM-STPP3-6 | Cluster 2.3/Cluster 2 |
| NO: 36 | | | | |
| SEQ ID | Polypeptide | Zea mays | ZM-STPP3-7 | Cluster 2.3/Cluster 2 |
| NO: 37 | | | | |
| SEQ ID | Polypeptide | Arabidopsis | AT-PP1 | Cluster 3.1/Cluster 3 |
| NO: 38 | | thaliana | | |
| SEQ ID | Polypeptide | Arabidopsis | AT-TOPP8-2 | Cluster 3.1/Cluster 3 |
| NO: 39 | | thaliana | | |
| SEQ ID | Polypeptide | Glycine max | GM-STPP-14 | Cluster 3.1/Cluster 3 |
| NO: 40 | | | | |
| SEQ ID | Polypeptide | Glycine max | GM-STPP-15 | Cluster 3.1/Cluster 3 |
| NO: 41 | | | | |
| SEQ ID | Polypeptide | Oryza sativa | OS-STPP3-5 | Cluster 3.2/Cluster 3 |
| NO: 42 | | | | |
| SEQ ID | Polypeptide | Sorghum | SB-STPP3-5 | Cluster 3.2/Cluster 3 |
| NO: 43 | | bicolor | | |
| SEQ ID | Polypeptide | Zea mays | ZM-STPP1 | Cluster 3.2/Cluster 3 |
| NO: 44 | | | | |
| SEQ ID | Polypeptide | Zea mays | ZM-STPP3-9 | Cluster 3.2/Cluster 3 |
| NO: 45 | | | | |
| SEQ ID | Polypeptide | Zea mays | ZM-STPP3-10 | Cluster 3.2/Cluster 3 |
| NO: 46 | | | | |
| SEQ ID | Polypeptide | Sorghum | SB-STPP3-6 | Cluster 3.2/Cluster 3 |
| NO: 47 | | bicolor | | |
| SEQ ID | Polynucleotide | Zea mays | ZM-STPP3 | Cluster 1.1/Cluster 1 |
| NO: 48 | | | | |
| SEQ ID | Polynucleotide | Zea mays | ZM-STPP3-2 | Cluster 1.1/Cluster 1 |
| NO: 49 | | | | |
| SEQ ID | Polynucleotide | Zea mays | ZM-STPP3-1 | Cluster 1.1/Cluster 1 |
| NO: 50 | | | | |
| SEQ ID | Polynucleotide | Sorghum | SB-STPP3-1 | Cluster 1.1/Cluster 1 |
| NO: 51 | | bicolor | | |
| SEQ ID | Polynucleotide | Oryza sativa | OS-STPP3-1 | Cluster 1.1/Cluster 1 |
| NO: 52 | | | | |
| SEQ ID | Polynucleotide | Arabidopsis | AT-TOPP4 | Cluster 1.1/Cluster 1 |
| NO: 53 | | thaliana | | |
| SEQ ID | Polynucleotide | Glycine max | GM-STPP-1 | Cluster 1.1/Cluster 1 |
| NO: 54 | | | | |
| SEQ ID | Polynucleotide | Glycine max | GM-STPP-2 | Cluster 1.1/Cluster 1 |
| NO: 55 | | | | |
| SEQ ID | Polynucleotide | Glycine max | GM-STPP-3 | Cluster 1.2/Cluster 1 |
| NO: 56 | | | | |
| SEQ ID | Polynucleotide | Glycine max | GM-STPP-4 | Cluster 1.2/Cluster 1 |
| NO: 57 | | | | |
| SEQ ID | Polynucleotide | Glycine max | GM-STPP-5 | Cluster 1.2/Cluster 1 |
| NO: 58 | | | | |
| SEQ ID | Polynucleotide | Glycine max | GM-STPP-6 | Cluster 1.2/Cluster 1 |
| NO: 59 | | | | |
| SEQ ID | Polynucleotide | Oryza sativa | OS-STPP3-2 | Cluster 1.2/Cluster 1 |
| NO: 60 | | | | |
| SEQ ID | Polynucleotide | Sorghum | SB-STPP3-2 | Cluster 1.2/Cluster 1 |
| NO: 61 | | bicolor | | |
| SEQ ID | Polynucleotide | Zea mays | ZM-STPP3-3 | Cluster 1.2/Cluster 1 |
| NO: 62 | | | | |
| SEQ ID | Polynucleotide | Zea mays | ZM-STPP3-4 | Cluster 1.2/Cluster 1 |
| NO: 63 | | | | |
| SEQ ID | Polynucleotide | Arabidopsis | AT-TOPP1 | Cluster 1.3/Cluster 1 |
| NO: 64 | | thaliana | | |
| SEQ ID | Polynucleotide | Arabidopsis | AT-TOPP5 | Cluster 1.3/Cluster 1 |
| NO: 65 | | thaliana | | |
| SEQ ID | Polynucleotide | Arabidopsis | AT-TOPP2 | Cluster 1.3/Cluster 1 |
| NO: 66 | | thaliana | | |
| SEQ ID | Polynucleotide | Arabidopsis | AT-TOPP7-1 | Cluster 1.4/Cluster 1 |
| NO: 67 | | thaliana | | |
| SEQ ID | Polynucleotide | Glycine max | GM-STPP-7 | Cluster 1.4/Cluster 1 |
| NO: 68 | | | | |
| SEQ ID | Polynucleotide | Glycine max | GM-STPP-8 | Cluster 1.4/Cluster 1 |
| NO: 69 | | | | |
| SEQ ID | Polynucleotide | Glycine max | GM-STPP-9 | Cluster 1.4/Cluster 1 |
| NO: 70 | | | | |
| SEQ ID | Polynucleotide | Glycine max | GM-STPP-10 | Cluster 1.4/Cluster 1 |
| NO: 71 | | | | |
| SEQ ID | Polynucleotide | Glycine max | GM-STPP-11 | Cluster 2.1/Cluster 2 |
| NO: 72 | | | | |
| SEQ ID | Polynucleotide | Glycine max | GM-STPP-12 | Cluster 2.1/Cluster 2 |
| NO: 73 | | | | |
| SEQ ID | Polynucleotide | Arabidopsis | AT-TOPP6 | Cluster 2.1/Cluster 2 |
| NO: 74 | | thaliana | | |
| SEQ ID | Polynucleotide | Arabidopsis | AT-TOPP3 | Cluster 2.1/Cluster 2 |
| NO: 75 | | thaliana | | |
| SEQ ID | Polynucleotide | Zea mays | ZM-STPP3-8 | Cluster 2.2/Cluster 2 |
| NO: 76 | | | | |
| SEQ ID | Polynucleotide | Sorghum | SB-STPP3-4 | Cluster 2.2/Cluster 2 |
| NO: 77 | | bicolor | | |
| SEQ ID | Polynucleotide | Oryza sativa | OS-STPP3-4 | Cluster 2.2/Cluster 2 |
| NO: 78 | | | | |
| SEQ ID | Polynucleotide | Glycine max | GM-STPP-13 | Cluster 2.2/Cluster 2 |
| NO: 79 | | | | |
| SEQ ID | Polynucleotide | Oryza sativa | OS-STPP3-3 | Cluster 2.3/Cluster 2 |
| NO: 80 | | | | |
| SEQ ID | Polynucleotide | Zea mays | ZM-STPP3-5 | Cluster 2.3/Cluster 2 |
| NO: 81 | | | | |
| SEQ ID | Polynucleotide | Sorghum | SB-STPP3-3 | Cluster 2.3/Cluster 2 |
| NO: 82 | | bicolor | | |
| SEQ ID | Polynucleotide | Zea mays | ZM-STPP3-6 | Cluster 2.3/Cluster 2 |
| NO: 83 | | | | |
| SEQ ID | Polynucleotide | Zea mays | ZM-STPP3-7 | Cluster 2.3/Cluster 2 |
| NO: 84 | | | | |
| SEQ ID | Polynucleotide | Arabidopsis | AT-PP1 | Cluster 3.1/Cluster 3 |
| NO: 85 | | thaliana | | |
| SEQ ID | Polynucleotide | Arabidopsis | AT-TOPP8-2 | Cluster 3.1/Cluster 3 |
| NO: 86 | | thaliana | | |
| SEQ ID | Polynucleotide | Glycine max | GM-STPP-14 | Cluster 3.1/Cluster 3 |
| NO: 87 | | | | |
| SEQ ID | Polynucleotide | Glycine max | GM-STPP-15 | Cluster 3.1/Cluster 3 |
| NO: 88 | | | | |
| SEQ ID | Polynucleotide | Oryza sativa | OS-STPP3-5 | Cluster 3.2/Cluster 3 |
| NO: 89 | | | | |
| SEQ ID | Polynucleotide | Sorghum | SB-STPP3-5 | Cluster 3.2/Cluster 3 |
| NO: 90 | | bicolor | | |
| SEQ ID | Polynucleotide | Zea mays | ZM-STPP1 | Cluster 3.2/Cluster 3 |
| NO: 91 | | | | |
| SEQ ID | Polynucleotide | Zea mays | ZM-STPP3-9 | Cluster 3.2/Cluster 3 |
| NO: 92 | | | | |
| SEQ ID | Polynucleotide | Zea mays | ZM-STPP3-10 | Cluster 3.2/Cluster 3 |
| NO: 93 | | | | |
| SEQ ID | Polynucleotide | Sorghum | SB-STPP3-6 | Cluster 3.2/Cluster 3 |
| NO: 94 | | bicolor | | |
| SEQ ID | Polypeptide | Artificial | Motif-N | N-Terminus Motif: |
| NO: 95 | | sequence | | L[L/T]EVR[T/L]ARPGK |
| | | | | QVQL |
| SEQ ID | Polypeptide | Artificial | Motif-C | C-Terminus Motif: |
| NO: 96 | | sequence | | GAMMSVDE[T/N]LMC |
| | | | | SFQ |
| SEQ ID | Polynucleotide | Pennisetum | PG-STPP3-1 | Cluster 1.1/Cluster 1 |
| NO: 97 | | glaucum | | |
| SEQ ID | Polynucleotide | Dennstaedtia | DP-STPP3-1 | Cluster 1.5/Cluster 1 |
| NO: 98 | | punctilobula | | |
| SEQ ID | Polynucleotide | Dennstaedtia | DP-STPP3-2 | Cluster 1.5/Cluster 1 |
| NO: 99 | | punctilobula | | |
| SEQ ID | Polynucleotide | Dennstaedtia | DP-STPP3-3 | Cluster 1.5/Cluster 1 |
| NO: 100 | | punctilobula | | |
| SEQ ID | Polynucleotide | Dennstaedtia | DP-STPP3-4 | Cluster 1.5/Cluster 1 |
| NO: 101 | | punctilobula | | |
| SEQ ID | Polynucleotide | Dennstaedtia | DP-STPP3-5 | Cluster 1.5/Cluster 1 |
| NO: 102 | | punctilobula | | |
| SEQ ID | Polynucleotide | Dennstaedtia | DP-STPP3-6 | Cluster 1.5/Cluster 1 |
| NO: 103 | | punctilobula | | |
| SEQ ID | Polypeptide | Pennisetum | PG-STPP3-1 | Cluster 1.1/Cluster 1 |
| NO: 104 | | glaucum | | |
| SEQ ID | Polypeptide | Dennstaedtia | DP-STPP3-1 | Cluster 1.5/Cluster 1 |
| NO: 105 | | punctilobula | | |
| SEQ ID | Polypeptide | Dennstaedtia | DP-STPP3-2 | Cluster 1.5/Cluster 1 |
| NO: 106 | | punctilobula | | |
| SEQ ID | Polypeptide | Dennstaedtia | DP-STPP3-3 | Cluster 1.5/Cluster 1 |
| NO: 107 | | punctilobula | | |
| SEQ ID | Polypeptide | Dennstaedtia | DP-STPP3-4 | Cluster 1.5/Cluster 1 |
| NO: 108 | | punctilobula | | |
| SEQ ID | Polypeptide | Dennstaedtia | DP-STPP3-5 | Cluster 1.5/Cluster 1 |
| NO: 109 | | punctilobula | | |
| SEQ ID | Polypeptide | Dennstaedtia | DP-STPP3-6 | Cluster 1.5/Cluster 1 |
| NO: 110 | | punctilobula | | |
| SEQ ID | Polypeptide | Paspalum | PN-STPP3-1 | Cluster 1.1/Cluster 1 |
| NO: 111 | | notatum | | |
| SEQ ID | Polynucleotide | Paspalum | PN-STPP3-1 | Cluster 1.1/Cluster 1 |
| NO: 112 | | notatum | | |
| SEQ ID | Polypeptide | Arabidopsis | AT-TOPP8-1 | Cluster 3.1/Cluster 3 |
| NO: 113 | | thaliana | | |
| SEQ ID | Polynucleotide | Arabidopsis | AT-TOPP8-1 | Cluster 3.1/Cluster 3 |
| NO: 114 | | thaliana | | |
| SEQ ID | Polypeptide | Arabidopsis | AT-TOPP7-2 | Cluster 1.4/Cluster 1 |
| NO: 115 | | thaliana | | |
| SEQ ID | Polynucleotide | Arabidopsis | AT-TOPP7-2 | Cluster 1.4/Cluster 1 |
| NO: 116 | | thaliana | | |
| SEQ ID | Polypeptide | Arabidopsis | AT-TOPP7-3 | Cluster 1.4/Cluster 1 |
| NO: 117 | | thaliana | | |
| SEQ ID | Polynucleotide | Arabidopsis | AT-TOPP7-3 | Cluster 1.4/Cluster 1 |
| NO: 118 | | thaliana | | |

### Construction of Nucleic Acids

The isolated nucleic acids of the present disclosure can be made using (a) standard recombinant methods, (b) synthetic techniques or combinations thereof. In some embodiments, the polynucleotides of the present disclosure will be cloned, amplified or otherwise constructed from a fungus or bacteria.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in essentially all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters may be “inducible” promoters. Environmental conditions that may effect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light. Diurnal promoters that are active at different times during the circadian rhythm are also known (US Patent Application Publication Number 2011/0167517, incorporated herein by reference).

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119 and hereby incorporated by reference) or signal peptides which target proteins to the plastids such as that of rapeseed enoyl-Acp reductase (Verwaert, et al., (1994) Plant Mol. Biol. 26:189-202) are useful in the disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the NUE gene placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert an NUE polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas, (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Once constructed, plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Cornpositae, and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Reducing the Activity and/or Level of a Polypeptide

Methods are provided to reduce or eliminate the activity of a polypeptide of the disclosure by transforming a plant cell with an expression cassette that expresses a polynucleotide that inhibits the expression of the polypeptide. The polynucleotide may inhibit the expression of the polypeptide directly, by preventing transcription or translation of the messenger RNA, or indirectly, by encoding a polypeptide that inhibits the transcription or translation of a gene encoding polypeptide. Methods for inhibiting or eliminating the expression of a gene in a plant are well known in the art and any such method may be used in the present disclosure to inhibit the expression of polypeptide.

In accordance with the present disclosure, the expression of polypeptide is inhibited if the protein level of the polypeptide is less than 70% of the protein level of the same polypeptide in a plant that has not been genetically modified or mutagenized to inhibit the expression of that polypeptide. In particular embodiments of the disclosure, the protein level of the polypeptide in a modified plant according to the disclosure is less than 60%, less than 50%, less than 40%, less than 30%, less than 20%, less than 10%, less than 5% or less than 2% of the protein level of the same polypeptide in a plant that is not a mutant or that has not been genetically modified to inhibit the expression of that polypeptide. The expression level of the polypeptide may be measured directly, for example, by assaying for the level of polypeptide expressed in the plant cell or plant, or indirectly, for example, by measuring the nitrogen uptake activity of the polypeptide in the plant cell or plant or by measuring the phenotypic changes in the plant. Methods for performing such assays are described elsewhere herein.

In other embodiments of the disclosure, the activity of the polypeptides is reduced or eliminated by transforming a plant cell with an expression cassette comprising a polynucleotide encoding a polypeptide that inhibits the activity of a polypeptide. The enhanced nitrogen utilization activity of a polypeptide is inhibited according to the present disclosure if the activity of the polypeptide is less than 70% of the activity of the same polypeptide in a plant that has not been modified to inhibit the activity of that polypeptide. In particular embodiments of the disclosure, the activity of the polypeptide in a modified plant according to the disclosure is less than 60%, less than 50%, less than 40%, less than 30%, less than 20%, less than 10% or less than 5% of the activity of the same polypeptide in a plant that that has not been modified to inhibit the expression of that polypeptide. The activity of a polypeptide is “eliminated” according to the disclosure when it is not detectable by the assay methods described elsewhere herein. Methods of determining the alteration of nitrogen utilization activity of a polypeptide are described elsewhere herein.

In other embodiments, the activity of a polypeptide may be reduced or eliminated by disrupting the gene encoding the polypeptide. The disclosure encompasses mutagenized plants that carry mutations in genes, where the mutations reduce expression of the gene or inhibit the nitrogen utilization activity of the encoded polypeptide.

Thus, many methods may be used to reduce or eliminate the activity of a polypeptide. In addition, more than one method may be used to reduce the activity of a single polypeptide.

1. Polynucleotide-Based Methods:

In some embodiments of the present disclosure, a plant is transformed with an expression cassette that is capable of expressing a polynucleotide that inhibits the expression of a polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one polypeptide is an expression cassette capable of producing an RNA molecule that inhibits the transcription and/or translation of at least one polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a polypeptide are given below.

i. Sense Suppression/Cosuppression

In some embodiments of the disclosure, inhibition of the expression of a polypeptide may be obtained by sense suppression or cosuppression. For cosuppression, an expression cassette is designed to express an RNA molecule corresponding to all or part of a messenger RNA encoding a polypeptide in the “sense” orientation. Over expression of the RNA molecule can result in reduced expression of the native gene. Accordingly, multiple plant lines transformed with the cosuppression expression cassette are screened to identify those that show the desired degree of inhibition of polypeptide expression.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In some embodiments where the polynucleotide comprises all or part of the coding region for the polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the polypeptide may be obtained by antisense suppression. For antisense suppression, the expression cassette is designed to express an RNA molecule complementary to all or part of a messenger RNA encoding the polypeptide. Over expression of the antisense RNA molecule can result in reduced expression of the target gene. Accordingly, multiple plant lines transformed with the antisense suppression expression cassette are screened to identify those that show the desired degree of inhibition of polypeptide expression.

The polynucleotide for use in antisense suppression may correspond to all or part of the complement of the sequence encoding the polypeptide, all or part of the complement of the 5′ and/or 3′ untranslated region of the target transcript or all or part of the complement of both the coding sequence and the untranslated regions of a transcript encoding the polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence.

Alternatively, separate expression cassettes may be used for the sense and antisense sequences. Multiple plant lines transformed with the dsRNA interference expression cassette or expression cassettes are then screened to identify plant lines that show the desired degree of inhibition of polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of a polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene whose expression is to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904; Mette, et al., (2000) EMBO J. 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the polypeptide. Thus, the polynucleotide causes the degradation of the endogenous messenger RNA, resulting in reduced expression of the polypeptide. This method is described, for example, in U.S. Pat. No. 4,987,071, herein incorporated by reference.

vii. Small Interfering RNA or Micro RNA

In some embodiments of the disclosure, inhibition of the expression of a polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. For example, the miRNA gene encodes an RNA that forms a hairpin structure containing a 22-nucleotide sequence that is complementary to another endogenous gene (target sequence). For suppression of NUE expression, the 22-nucleotide sequence is selected from a NUE transcript sequence and contains 22 nucleotides of said NUE sequence in sense orientation and 21 nucleotides of a corresponding antisense sequence that is complementary to the sense sequence. A fertility gene, whether endogenous or exogenous, may be an miRNA target. miRNA molecules are highly efficient at inhibiting the expression of endogenous genes, and the RNA interference they induce is inherited by subsequent generations of plants.

2. Polypeptide-Based Inhibition of Gene Expression

In one embodiment, the polynucleotide encodes a zinc finger protein that binds to a gene encoding a polypeptide, resulting in reduced expression of the gene. In particular embodiments, the zinc finger protein binds to a regulatory region of a NUE gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one polypeptide and reduces the enhanced nitrogen utilization activity of the polypeptide. In another embodiment, the binding of the antibody results in increased turnover of the antibody-NUE complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of a polypeptide is reduced or eliminated by disrupting the gene encoding the polypeptide. The gene encoding the polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced nitrogen utilization activity.

i. Transposon Tagging

In one embodiment of the disclosure, transposon tagging is used to reduce or eliminate the activity of one or more polypeptide. Transposon tagging comprises inserting a transposon within an endogenous NUE gene to reduce or eliminate expression of the polypeptide. “NUE gene” is intended to mean the gene that encodes a polypeptide according to the disclosure.

In this embodiment, the expression of one or more polypeptide is reduced or eliminated by inserting a transposon within a regulatory region or coding region of the gene encoding the polypeptide. A transposon that is within an exon, intron, 5′ or 3′ untranslated sequence, a promoter or any other regulatory sequence of a NUE gene may be used to reduce or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function (enhanced nitrogen utilization activity) of the encoded protein are well known in the art. Insertional mutations in gene exons usually result in null-mutants. Mutations in conserved residues are particularly effective in inhibiting the activity of the encoded protein. Conserved residues of plant polypeptides suitable for mutagenesis with the goal to eliminate activity have been described. Such mutants can be isolated according to well-known procedures and mutations in different NUE loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Nitrogen Utilization Activity

In specific methods, the level and/or activity of a NUE regulator in a plant is decreased by increasing the level or activity of the polypeptide in the plant. The increased expression of a negative regulatory molecule may decrease the level of expression of downstream one or more genes responsible for an improved NUE phenotype.

Methods for increasing the level and/or activity of polypeptides in a plant are discussed elsewhere herein. Briefly, such methods comprise providing a polypeptide of the disclosure to a plant and thereby increasing the level and/or activity of the polypeptide. In other embodiments, a NUE nucleotide sequence encoding a polypeptide can be provided by introducing into the plant a polynucleotide comprising a NUE nucleotide sequence of the disclosure, expressing the NUE sequence, increasing the activity of the polypeptide and thereby decreasing the number of tissue cells in the plant or plant part. In other embodiments, the NUE nucleotide construct introduced into the plant is stably incorporated into the genome of the plant.

In other methods, the growth of a plant tissue is increased by decreasing the level and/or activity of the polypeptide in the plant. Such methods are disclosed in detail elsewhere herein. In one such method, a NUE nucleotide sequence is introduced into the plant and expression of said NUE nucleotide sequence decreases the activity of the polypeptide and thereby increasing the tissue growth in the plant or plant part. In other embodiments, the NUE nucleotide construct introduced into the plant is stably incorporated into the genome of the plant.

As discussed above, one of skill will recognize the appropriate promoter to use to modulate the level/activity of a NUE in the plant. Exemplary promoters for this embodiment have been disclosed elsewhere herein.

In other embodiments, such plants have stably incorporated into their genome a nucleic acid molecule comprising a NUE nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the polypeptide in the plant. In one method, a NUE sequence of the disclosure is provided to the plant. In another method, the NUE nucleotide sequence is provided by introducing into the plant a polynucleotide comprising a NUE nucleotide sequence of the disclosure, expressing the NUE sequence and thereby modifying root development. In still other methods, the NUE nucleotide construct introduced into the plant is stably incorporated into the genome of the plant.

In other methods, root development is modulated by altering the level or activity of the polypeptide in the plant. A change in activity can result in at least one or more of the following alterations to root development, including, but not limited to, alterations in root biomass and length.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by decreasing the activity and/or level of the polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by altering the level and/or activity of the polypeptide also finds use in promoting in vitro propagation of explants.

Furthermore, higher root biomass production due to activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the polypeptide of the disclosure and has enhanced root growth and/or root biomass. In other embodiments, such plants have stably incorporated into their genome a nucleic acid molecule comprising a NUE nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a polypeptide of the disclosure. In one embodiment, a NUE sequence of the disclosure is provided. In other embodiments, the NUE nucleotide sequence can be provided by introducing into the plant a polynucleotide comprising a NUE nucleotide sequence of the disclosure, expressing the NUE sequence and thereby modifying shoot and/or leaf development. In other embodiments, the NUE nucleotide construct introduced into the plant is stably incorporated into the genome of the plant.

In specific embodiments, shoot or leaf development is modulated by altering the level and/or activity of the polypeptide in the plant. A change in activity can result in at least one or more of the following alterations in shoot and/or leaf development, including, but not limited to, changes in leaf number, altered leaf surface, altered vasculature, internodes and plant growth and alterations in leaf senescence when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Increasing activity and/or level in a plant results in altered internodes and growth. Thus, the methods of the disclosure find use in producing modified plants. In addition, as discussed above, activity in the plant modulates both root and shoot growth. Thus, the present disclosure further provides methods for altering the root/shoot ratio. Shoot or leaf development can further be modulated by altering the level and/or activity of the polypeptide in the plant.

Accordingly, the present disclosure further provides plants having modulated shoot and/or leaf development when compared to a control plant. In some embodiments, the plant of the disclosure has an increased level/activity of the polypeptide of the disclosure. In other embodiments, the plant of the disclosure has a decreased level/activity of the polypeptide of the disclosure.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the polypeptide has not been modulated. “Modulating floral development” further includes any alteration in the timing of the development of a plant's reproductive tissue (i.e., a delayed or an accelerated timing of floral development) when compared to a control plant in which the activity or level of the polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating activity in a plant. In one method, a NUE sequence of the disclosure is provided. A NUE nucleotide sequence can be provided by introducing into the plant a polynucleotide comprising a NUE nucleotide sequence of the disclosure, expressing the NUE sequence and thereby modifying floral development. In other embodiments, the NUE nucleotide construct introduced into the plant is stably incorporated into the genome of the plant.

In specific methods, floral development is modulated by increasing the level or activity of the polypeptide in the plant. A change in activity can result in at least one or more of the following alterations in floral development, including, but not limited to, altered flowering, changed number of flowers, modified male sterility and altered seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by altering the level and/or activity of the NUE sequence of the disclosure. Such methods can comprise introducing a NUE nucleotide sequence into the plant and changing the activity of the polypeptide. In other methods, the NUE nucleotide construct introduced into the plant is stably incorporated into the genome of the plant. Altering expression of the NUE sequence of the disclosure can modulate floral development during periods of stress. Such methods are described elsewhere herein. Accordingly, the present disclosure further provides plants having modulated floral development when compared to the floral development of a control plant. Compositions include plants having an altered level/activity of the polypeptide of the disclosure and having an altered floral development. Compositions also include plants having a modified level/activity of the polypeptide of the disclosure wherein the plant maintains or proceeds through the flowering process in times of stress.

Methods are also provided for the use of the NUE sequences of the disclosure to increase seed size and/or weight. The method comprises increasing the activity of the NUE sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for altering seed size and/or seed weight in a plant comprises increasing activity in the plant. In one embodiment, the NUE nucleotide sequence can be provided by introducing into the plant a polynucleotide comprising a NUE nucleotide sequence of the disclosure, expressing the NUE sequence and thereby decreasing seed weight and/or size. In other embodiments, the NUE nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has a modified level/activity of the polypeptide of the disclosure and has an increased seed weight and/or seed size. In other embodiments, such plants have stably incorporated into their genome a nucleic acid molecule comprising a NUE nucleotide sequence of the disclosure operably linked to a promoter that drives expression in the plant cell.

vii. Method of Use for NUE Polynucleotide, Expression Cassettes, and Additional Polynucleotides

The nucleotides, expression cassettes and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference. Known genes that confer tolerance to herbicides such as e.g., auxin, HPPD, glyphosate, dicamba, glufosinate, sulfonylurea, bromoxynil and norflurazon herbicides can be stacked either as a molecular stack or a breeding stack with plants expressing the traits disclosed herein. Polynucleotide molecules encoding proteins involved in herbicide tolerance include, but are not limited to, a polynucleotide molecule encoding 5-enolpyruvylshikimate-3-phosphate synthase (EPSPS) disclosed in U.S. Pat. Nos. 39,247; 6,566,587 and for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Pat. Nos. 7,622,641; 7,462,481; 7,531,339; 7,527,955; 7,709,709; 7,714,188 and 7,666,643, also for providing glyphosate tolerance; dicamba monooxygenase disclosed in U.S. Pat. No. 7,022,896 and WO 2007/146706 A2 for providing dicamba tolerance; a polynucleotide molecule encoding AAD12 disclosed in US Patent Application Publication Number 2005/731044 or WO 2007/053482 A2 or encoding AAD1 disclosed in US Patent Application Publication Number 2011/0124503 A1 or U.S. Pat. No. 7,838,733 for providing tolerance to auxin herbicides (2,4-D); a polynucleotide molecule encoding hydroxyphenylpyruvate dioxygenase (HPPD) for providing tolerance to HPPD inhibitors (e.g., hydroxyphenylpyruvate dioxygenase) disclosed in e.g., U.S. Pat. No. 7,935,869; US Patent Application Publication Numbers 2009/0055976 A1 and 2011/0023180 A1, each publication is herein incorporated by reference in its entirety.

Other examples of herbicide-tolerance traits that could be combined with the traits disclosed herein include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616 and 5,879,903. Plants containing an exogenous phosphinothricin acetyltransferase can exhibit improved tolerance to glufosinate herbicides, which inhibit the enzyme glutamine synthase. Other examples of herbicide-tolerance traits include those conferred by polynucleotides conferring altered protoporphyrinogen oxidase (protox) activity, as described in U.S. Pat. Nos. 6,288,306 B1; 6,282,837 B1 and 5,767,373 and international publication WO 2001/12825. Plants containing such polynucleotides can exhibit improved tolerance to any of a variety of herbicides which target the protox enzyme (also referred to as “protox inhibitors”)

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem. 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley described in Williamson, et al., (1987) Eur. J. Biochem. 165:99-106, the disclosures of which are herein incorporated by reference.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109) and the like.

Commercial traits can also be encoded on a gene or genes that could increase for example, starch for ethanol production or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

The promoter, which is operably linked to the nucleotide sequence, can be any promoter that is active in plant cells, particularly a promoter that is active (or can be activated) in reproductive tissues of a plant (e.g., stamens or ovaries). As such, the promoter can be, for example, a constitutively active promoter, an inducible promoter, a tissue-specific promoter or a developmental stage specific promoter. Also, the promoter of the first exogenous nucleic acid molecule can be the same as or different from the promoter of the second exogenous nucleic acid molecule.

In general, a promoter is selected based, for example, on whether endogenous fertility genes to be inhibited are male fertility genes or female fertility genes. Thus, where the endogenous genes to be inhibited are male fertility genes (e.g., a BS7 gene and an SB200 gene), the promoter can be a stamen specific and/or pollen specific promoter such as an MS45 gene promoter (U.S. Pat. No. 6,037,523), a 5126 gene promoter (U.S. Pat. No. 5,837,851), a BS7 gene promoter (WO 2002/063021), an SB200 gene promoter (WO 2002/26789), a TA29 gene promoter (Nature 347:737 (1990)), a PG47 gene promoter (U.S. Pat. No. 5,412,085; U.S. Pat. No. 5,545,546; Plant J 3(2):261-271 (1993)) an SGB6 gene promoter (U.S. Pat. No. 5,470,359) a G9 gene promoter (U.S. Pat. Nos. 5,837,850 and 5,589,610) or the like, such that the hpRNA is expressed in anther and/or pollen or in tissues that give rise to anther cells and/or pollen, thereby reducing or inhibiting expression of the endogenous male fertility genes (i.e., inactivating the endogenous male fertility genes). In comparison, where the endogenous genes to be inhibited are female fertility genes, the promoter can be an ovary specific promoter, for example. However, as disclosed herein, any promoter can be used that directs expression in the tissue of interest, including, for example, a constitutively active promoter such as an ubiquitin promoter, which generally effects transcription in most or all plant cells.

Genome Editing and Induced Mutagenesis

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” meganucleases produced to modify plant genomes (see, e.g., WO 2009/114321; Gao, et al., (2010) Plant Journal 1:176-187). Another site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41.

“TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484). Methods for TILLING are well known in the art (U.S. Pat. No. 8,071,840).

Other mutagenic methods can also be employed to introduce mutations in the STPP gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Exemplary constitutive promoters include the 35S cauliflower mosaic virus (CaMV) promoter promoter (Odell, et al., (1985) Nature 313:810-812), the maize ubiquitin promoter (Christensen, et al., (1989) Plant Mol. Biol. 12:619-632 and Christensen, et al., (1992) Plant Mol. Biol. 18:675-689); the core promoter of the Rsyn7 promoter and other constitutive promoters disclosed in WO 1999/43838 and U.S. Pat. No. 6,072,050; rice actin (McElroy, et al., (1990) Plant Cell 2:163-171); pEMU (Last, et al., (1991) Theor. Appl. Genet. 81:581-588); MAS (Velten, et al., (1984) EMBO J. 3:2723-2730); ALS promoter (U.S. Pat. No. 5,659,026); rice actin promoter (U.S. Pat. No. 5,641,876; WO 2000/70067), maize histone promoter (Brignon, et al., (1993) Plant Mol Bio 22(6):1007-1015; Rasco-Gaunt, et al., (2003) Plant Cell Rep. 21(6):569-576) and the like. Other constitutive promoters include, for example, those described in U.S. Pat. Nos. 5,608,144 and 6,177,611 and PCT Publication Number WO 2003/102198.

Tissue-specific, tissue-preferred or stage-specific regulatory elements further include, for example, the AGL8/FRUITFULL regulatory element, which is activated upon floral induction (Hempel, et al., (1997) Development 124:3845-3853); root-specific regulatory elements such as the regulatory elements from the RCP1 gene and the LRP1 gene (Tsugeki and Fedoroff, (1999) Proc. Natl. Acad., USA 96:12941-12946; Smith and Fedoroff, (1995) Plant Cell 7:735-745); flower-specific regulatory elements such as the regulatory elements from the LEAFY gene and the APETALA1 gene (Blazquez, et al., (1997) Development 124:3835-3844; Hempel, et al., supra, 1997); seed-specific regulatory elements such as the regulatory element from the oleosin gene (Plant, et al., (1994) Plant Mol. Biol. 25:193-205) and dehiscence zone specific regulatory element. Additional tissue-specific or stage-specific regulatory elements include the Zn13 promoter, which is a pollen-specific promoter (Hamilton, et al., (1992) Plant Mol. Biol. 18:211-218); the UNUSUAL FLORAL ORGANS (UFO) promoter, which is active in apical shoot meristem; the promoter active in shoot meristems (Atanassova, et al., (1992) Plant J. 2:291), the cdc2 promoter and cyc07 promoter (see, for example, Ito, et al., (1994) Plant Mol. Biol. 24:863-878; Martinez, et al., (1992) Proc. Natl. Acad. Sci., USA 89:7360); the meristematic-preferred meri-5 and H3 promoters (Medford, et al., (1991) Plant Cell 3:359; Terada, et al., (1993) Plant J. 3:241); meristematic and phloem-preferred promoters of Myb-related genes in barley (Wissenbach, et al., (1993) Plant J. 4:411); Arabidopsis cyc3aAt and cyc1At (Shaul, et al., (1996) Proc. Natl. Acad. Sci. 93:4868-4872); C. roseus cyclins CYS and CYM (Ito, et al., (1997) Plant J. 11:983-992); and Nicotiana CyclinB1 (Trehin, et al., (1997) Plant Mol. Biol. 35:667-672); the promoter of the APETALA3 gene, which is active in floral meristems (Jack, et al., (1994) Cell 76:703; Hempel, et al., supra, 1997); a promoter of an agamous-like (AGL) family member, for example, AGL8, which is active in shoot meristem upon the transition to flowering (Hempel, et al., supra, 1997); floral abscission zone promoters; L1-specific promoters; the ripening-enhanced tomato polygalacturonase promoter (Nicholass, et al., (1995) Plant Mol. Biol. 28:423-435), the E8 promoter (Deikman, et al., (1992) Plant Physiol. 100:2013-2017) and the fruit-specific 2A1 promoter, U2 and U5 snRNA promoters from maize, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD zein protein, and the like. Additional tissue-specific promoters can be isolated using well known methods (see, e.g., U.S. Pat. No. 5,589,379). Shoot-preferred promoters include shoot meristem-preferred promoters such as promoters disclosed in Weigel, et al., (1992) Cell 69:843-859 (Accession Number M91208); Accession Number AJ131822; Accession Number Z71981; Accession Number AF049870 and shoot-preferred promoters disclosed in McAvoy, et al., (2003) Acta Hort. (ISHS) 625:379-385. Inflorescence-preferred promoters include the promoter of chalcone synthase (Van der Meer, et al., (1992) Plant J. 2(4):525-535), anther-specific LAT52 (Twell, et al., (1989) Mol. Gen. Genet. 217:240-245), pollen-specific Bp4 (Albani, et al., (1990) Plant Mol. Biol. 15:605, maize pollen-specific gene Zm13 (Hamilton, et al., (1992) Plant Mol. Biol. 18:211-218; Guerrero, et al., (1993) Mol. Gen. Genet. 224:161-168), microspore-specific promoters such as the apg gene promoter (Twell, et al., (1993) Sex. Plant Reprod. 6:217-224) and tapetum-specific promoters such as the TA29 gene promoter (Mariani, et al., (1990) Nature 347:737; U.S. Pat. No. 6,372,967) and other stamen-specific promoters such as the MS45 gene promoter, 5126 gene promoter, BS7 gene promoter, PG47 gene promoter (U.S. Pat. No. 5,412,085; U.S. Pat. No. 5,545,546; Plant J 3(2):261-271 (1993)), SGB6 gene promoter (U.S. Pat. No. 5,470,359), G9 gene promoter (U.S. Pat. No. 5,8937,850; U.S. Pat. No. 5,589,610), SB200 gene promoter (WO 2002/26789), or the like (see, Example 1). Tissue-preferred promoters of interest further include a sunflower pollen-expressed gene SF3 (Baltz, et al., (1992) The Plant Journal 2:713-721), B. napus pollen specific genes (Arnoldo, et al., (1992) J. Cell. Biochem, Abstract Number Y101204). Tissue-preferred promoters further include those reported by Yamamoto, et al., (1997) Plant J. 12(2):255-265 (psaDb); Kawamata, et al., (1997) Plant Cell Physiol. 38(7):792-803 (PsPAL1); Hansen, et al., (1997) Mol. Gen. Genet. 254(3):337-343 (ORF13); Russell, et al., (1997) Transgenic Res. 6(2):157-168 (waxy or ZmGBS; 27 kDa zein, ZmZ27; osAGP; osGT1); Rinehart, et al., (1996) Plant Physiol. 112(3):1331-1341 (FbI2A from cotton); Van Camp, et al., (1996) Plant Physiol. 112(2):525-535 (Nicotiana SodA1 and SodA2); Canevascini, et al., (1996) Plant Physiol. 112(2):513-524 (Nicotiana Itp1); Yamamoto, et al., (1994) Plant Cell Physiol. 35(5):773-778 (Pinus cab-6 promoter); Lam, (1994) Results Probl. Cell Differ. 20:181-196; Orozco, et al., (1993) Plant Mol. Biol. 23(6):1129-1138 (spinach rubisco activase (Rca)); Matsuoka, et al., (1993) Proc Natl. Acad. Sci. USA 90(20):9586-9590 (PPDK promoter) and Guevara-Garcia, et al., (1993) Plant J. 4(3):495-505 (Agrobacterium pmas promoter). A tissue-specific promoter that is active in cells of male or female reproductive organs can be particularly useful in certain aspects of the present disclosure.

“Seed-preferred” promoters include both “seed-developing” promoters (those promoters active during seed development such as promoters of seed storage proteins) as well as “seed-germinating” promoters (those promoters active during seed germination). See, Thompson, et al., (1989) BioEssays 10:108. Such seed-preferred promoters include, but are not limited to, Cim1 (cytokinin-induced message), cZ19B1 (maize 19 kDa zein), mi1ps (myo-inositol-1-phosphate synthase); see, WO 2000/11177 and U.S. Pat. No. 6,225,529. Gamma-zein is an endosperm-specific promoter. Globulin-1 (Glob-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also, WO 2000/12733 and U.S. Pat. No. 6,528,704, where seed-preferred promoters from end1 and end2 genes are disclosed. Additional embryo specific promoters are disclosed in Sato, et al., (1996) Proc. Natl. Acad. Sci. 93:8117-8122 (rice homeobox, OSH1) and Postma-Haarsma, et al., (1999) Plant Mol. Biol. 39:257-71 (rice KNOX genes). Additional endosperm specific promoters are disclosed in Albani, et al., (1984) EMBO 3:1405-15; Albani, et al., (1999) Theor. Appl. Gen. 98:1253-62; Albani, et al., (1993) Plant J. 4:343-55; Mena, et al., (1998) The Plant Journal 116:53-62 (barley DOF); Opsahl-Ferstad, et al., (1997) Plant J 12:235-46 (maize Esr) and Wu, et al., (1998) Plant Cell Physiology 39:885-889 (rice GluA-3, GluB-1, NRP33, RAG-1).

An inducible regulatory element is one that is capable of directly or indirectly activating transcription of one or more DNA sequences or genes in response to an inducer. The inducer can be a chemical agent such as a protein, metabolite, growth regulator, herbicide or phenolic compound or a physiological stress, such as that imposed directly by heat, cold, salt, or toxic elements or indirectly through the action of a pathogen or disease agent such as a virus or other biological or physical agent or environmental condition. A plant cell containing an inducible regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression. Typically the protein factor that binds specifically to an inducible regulatory element to activate transcription is present in an inactive form which is then directly or indirectly converted to the active form by the inducer. Any inducible promoter can be used in the instant disclosure (See, Ward, et al., (1993) Plant Mol. Biol. 22:361-366).

Examples of inducible regulatory elements include a metallothionein regulatory element, a copper-inducible regulatory element or a tetracycline-inducible regulatory element, the transcription from which can be effected in response to divalent metal ions, copper or tetracycline, respectively (Furst, et al., (1988) Cell 55:705-717; Mett, et al., (1993) Proc. Natl. Acad. Sci., USA 90:4567-4571; Gatz, et al., (1992) Plant J. 2:397-404; Roder, et al., (1994) Mol. Gen. Genet. 243:32-38). Inducible regulatory elements also include an ecdysone regulatory element or a glucocorticoid regulatory element, the transcription from which can be effected in response to ecdysone or other steroid (Christopherson, et al., (1992) Proc. Natl. Acad. Sci., USA 89:6314-6318; Schena, et al., (1991) Proc. Natl. Acad. Sci. USA 88:10421-10425; U.S. Pat. No. 6,504,082); a cold responsive regulatory element or a heat shock regulatory element, the transcription of which can be effected in response to exposure to cold or heat, respectively (Takahashi, et al., (1992) Plant Physiol. 99:383-390); the promoter of the alcohol dehydrogenase gene (Gerlach, et al., (1982) PNAS USA 79:2981-2985; Walker, et al., (1987) PNAS 84(19):6624-6628), inducible by anaerobic conditions; and the light-inducible promoter derived from the pea rbcS gene or pea psaDb gene (Yamamoto, et al., (1997) Plant J. 12(2):255-265); a light-inducible regulatory element (Feinbaum, et al., (1991) Mol. Gen. Genet. 226:449; Lam and Chua, (1990) Science 248:471; Matsuoka, et al., (1993) Proc. Natl. Acad. Sci. USA 90(20):9586-9590; Orozco, et al., (1993) Plant Mol. Bio. 23(6):1129-1138), a plant hormone inducible regulatory element (Yamaguchi-Shinozaki, et al., (1990) Plant Mol. Biol. 15:905; Kares, et al., (1990) Plant Mol. Biol. 15:225), and the like. An inducible regulatory element also can be the promoter of the maize In2-1 or In2-2 gene, which responds to benzenesulfonamide herbicide safeners (Hershey, et al., (1991) Mol. Gen. Gene. 227:229-237; Gatz, et al., (1994) Mol. Gen. Genet. 243:32-38) and the Tet repressor of transposon Tn10 (Gatz, et al., (1991) Mol. Gen. Genet. 227:229-237). Stress inducible promoters include salt/water stress-inducible promoters such as P5CS (Zang, et al., (1997) Plant Sciences 129:81-89); cold-inducible promoters, such as, cor15a (Hajela, et al., (1990) Plant Physiol. 93:1246-1252), cor15b (Wlihelm, et al., (1993) Plant Mol Biol 23:1073-1077), wsc120 (Ouellet, et al., (1998) FEBS Lett. 423:324-328), ci7 (Kirch, et al., (1997) Plant Mol. Biol. 33:897-909), ci21A (Schneider, et al., (1997) Plant Physiol. 113:335-45); drought-inducible promoters, such as, Trg-31 (Chaudhary, et al., (1996) Plant Mol. Biol. 30:1247-57), rd29 (Kasuga, et al., (1999) Nature Biotechnology 18:287-291); osmotic inducible promoters, such as Rab17 (Vilardell, et al., (1991) Plant Mol. Biol. 17:985-93) and osmotin (Raghothama, et al., (1993) Plant Mol Biol 23:1117-28) and heat inducible promoters, such as heat shock proteins (Barros, et al., (1992) Plant Mol. 19:665-75; Marrs, et al., (1993) Dev. Genet. 14:27-41), smHSP (Waters, et al., (1996) J. Experimental Botany 47:325-338) and the heat-shock inducible element from the parsley ubiquitin promoter (WO 2003/102198). Other stress-inducible promoters include rip2 (U.S. Pat. No. 5,332,808 and US Patent Application Publication Number 2003/0217393) and rd29a (Yamaguchi-Shinozaki, et al., (1993) Mol. Gen. Genetics 236:331-340). Certain promoters are inducible by wounding, including the Agrobacterium pmas promoter (Guevara-Garcia, et al., (1993) Plant J. 4(3):495-505) and the Agrobacterium ORF13 promoter (Hansen, et al., (1997) Mol. Gen. Genet. 254(3):337-343).

Plants suitable for purposes of the present disclosure can be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis thaliana and woody plants such as coniferous and deciduous trees. Thus, a transgenic plant or genetically modified plant cell of the disclosure can be an angiosperm or gymnosperm.

Cereal plants, which produce an edible grain, include, for example, corn, rice, wheat, barley, oat, rye, orchardgrass, guinea grass and sorghum. Leguminous plants include members of the pea family (Fabaceae) and produce a characteristic fruit known as a legume. Examples of leguminous plants include, for example, soybean, pea, chickpea, moth bean, broad bean, kidney bean, lima bean, lentil, cowpea, dry bean and peanut, as well as alfalfa, birdsfoot trefoil, clover and sainfoin. Oilseed plants, which have seeds that are useful as a source of oil, include soybean, sunflower, rapeseed (canola) and cottonseed. Angiosperms also include hardwood trees, which are perennial woody plants that generally have a single stem (trunk). Examples of such trees include alder, ash, aspen, basswood (linden), beech, birch, cherry, cottonwood, elm, eucalyptus, hickory, locust, maple, oak, persimmon, poplar, sycamore, walnut, sequoia and willow. Trees are useful, for example, as a source of pulp, paper, structural material and fuel.

Homozygosity is a genetic condition existing when identical alleles reside at corresponding loci on homologous chromosomes. Heterozygosity is a genetic condition existing when different alleles reside at corresponding loci on homologous chromosomes. Hemizygosity is a genetic condition existing when there is only one copy of a gene (or set of genes) with no allelic counterpart on the sister chromosome.

The plant breeding methods used herein are well known to one skilled in the art. For a discussion of plant breeding techniques, see, Poehlman, (1987) Breeding Field Crops AVI Publication Co., Westport Conn. Many of the plants which would be most preferred in this method are bred through techniques that take advantage of the plant's method of pollination.

Backcrossing methods may be used to introduce a gene into the plants. This technique has been used for decades to introduce traits into a plant. An example of a description of this and other plant breeding methodologies that are well known can be found in references such as Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

By transgene, it is meant any nucleic acid sequence which is introduced into the genome of a cell by genetic engineering techniques. A transgene may be a native DNA sequence or a heterologous DNA sequence (i.e., “foreign DNA”). The term native DNA sequence refers to a nucleotide sequence which is naturally found in the cell but that may have been modified from its original form.

Using well-known techniques, additional promoter sequences may be isolated based on their sequence homology. In these techniques, all or part of a known promoter sequence is used as a probe which selectively hybridizes to other sequences present in a population of cloned genomic DNA fragments (i.e. genomic libraries) from a chosen organism. Methods that are readily available in the art for the hybridization of nucleic acid sequences may be used to obtain sequences which correspond to these promoter sequences in species including, but not limited to, maize (corn; Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals and conifers. Preferably, plants include maize, soybean, sunflower, safflower, canola, wheat, barley, rye, alfalfa and sorghum.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see e.g., Innis, et al., (1990) PCR Protocols, A Guide to Methods and Applications, eds., Academic Press).

In general, sequences that correspond to a promoter sequence of the present disclosure and hybridize to a promoter sequence disclosed herein will be at least 50% homologous, 55% homologous, 60% homologous, 65% homologous, 70% homologous, 75% homologous, 80% homologous, 85% homologous, 90% homologous, 95% homologous and even 98% homologous or more with the disclosed sequence.

Fragments of a particular promoter sequence can be used to drive the expression of a gene of interest. These fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequences disclosed herein. The nucleotides of such fragments will usually comprise the TATA recognition sequence of the particular promoter sequence. Such fragments can be obtained by use of restriction enzymes to cleave the naturally-occurring promoter sequences disclosed herein; by synthesizing a nucleotide sequence from the naturally-occurring DNA sequence or through the use of PCR technology. See particularly, Mullis, et al., (1987) Methods Enzymol. 155:335-350 and Erlich, ed. (1989) PCR Technology (Stockton Press, New York). Again, variants of these fragments, such as those resulting from site-directed mutagenesis, are encompassed by the compositions of the present disclosure.

The nucleotide sequence operably linked to the regulatory elements disclosed herein can be an antisense sequence for a targeted gene. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences claimed herein can be operably linked to antisense DNA sequences to reduce or inhibit expression of a native or exogenous protein in the plant.

### EXAMPLES

### Example 1

### Creation of an Arabidopsis Population

A T-DNA based binary construct was created, containing four multimerized enhancer elements derived from the Cauliflower Mosaic Virus 35S promoter. The construct also contains vector sequences (pUC9) to allow plasmid rescue, transposon sequences (Ds) to remobilize the T-DNA and the bar gene to allow for glufosinate selection of transgenic plants. The enhancer elements can induce cis-activation of genomic loci following DNA integration in the genome. Arabidopsis plants were transformed and the population of Arabidopsis plants carrying enhancer elements were generated for further analysis.

A total of 100,000 glufosinate resistant T₁ seedlings were selected. T₂ seeds from each line were kept separate.

### Example 2

### Screens to Identify Lines with Altered Root Architecture

Activation-tagged Arabidopsis seedlings, grown under non-limiting nitrogen conditions, were analyzed for altered root system architecture when compared to control seedlings during early development from the population described in Example 1.

Validated leads from in-house screen were subjected to a vertical plate assay to evaluate enhanced root growth. The results were validated using WinRHIZO®, as described below. T1 or T2 seeds were sterilized using 50% household bleach 0.01% triton X-100 solution and plated on petri plates containing the following medium: 0.5×N-Free Hoagland's, 60 mM KNO₃, 0.1% sucrose, 1 mM MES and 1% Phytagel™ at a density of 4 seeds/plate or 0.5×N-Free Hoagland's, 4 mM KNO₃, 1% sucrose, 1 mM MES and 1% Phytagel™ at a density of 4 seeds/plate. Plates were kept for three days at 4° C. to stratify seeds and then held vertically for 11 days at 22° C. light and 20° C. dark. Photoperiod was 16 h; 8 h dark and average light intensity was ˜160 pmol/m²/s. Plates were placed vertically into the eight center positions of a 10 plate rack with the first and last position holding blank plates. The racks and the plates within a rack were rotated every other day. Two sets of pictures were taken for each plate. The first set taking place at day 14-16 when the primary roots for most lines had reached the bottom of the plate, the second set of pictures two days later after more lateral roots had developed. The latter set of picture was usually used for data analysis. These seedlings grown on vertical plates were analyzed for root growth with the software WinRHIZO® (Regent Instruments Inc), an image analysis system specifically designed for root measurement. WinRHIZO® uses the contrast in pixels to distinguish the light root from the darker background. To identify the maximum amount of roots without picking up background, the pixel classification was 150-170 and the filter feature was used to remove objects that have a length/width ratio less then 10.0. The area on the plates analyzed was from the edge of the plant's leaves to about 1 cm from the bottom of the plate. The exact same WinRHIZO® settings and area of analysis were used to analyze all plates within a batch. The total root length score given by WinRHIZO® for a plate was divided by the number of plants that had germinated and had grown halfway down the plate. Eight plates for every line were grown and their scores were averaged. This average was then compared to the average of eight plates containing wild type seeds that were grown at the same time.

Lines with enhanced root growth characteristics were expected to lie at the upper extreme of the root area distributions. A sliding window approach was used to estimate the variance in root area for a given rack with the assumption that there could be up to two outliers in the rack. Environmental variations in various factors including growth media, temperature and humidity can cause significant variation in root growth, especially between sow dates. Therefore the lines were grouped by sow date and shelf for the data analysis. The racks in a particular sow date/shelf group were then sorted by mean root area. Root area distributions for sliding windows were performed by combining data for a rack, rᵢ, with data from the rack with the next lowest, (rᵢ₋₁, and the next highest mean root area, rᵢ₊₁. The variance of the combined distribution was then analyzed to identify outliers in rᵢ using a Grubbs-type approach (Barnett, et al., Outliers in Statistical Data, John Wiley & Sons, 3^{rd} edition (1994).

T1 transgenic plants overexpressing individually ZmSTPP3, AtPP1 or other AtTOPP family members were evaluated in this assay. Transgenic plants overexpressing each of these sequences (ZmSTPP3, SEQ ID NO: 48; AtTOPP4, SEQ ID NO: 53; AtTOPP2, SEQ ID NO: 66; and AtTOPP8, SEQ ID NO: 86 and SEQ ID NO: 114) showed improved root growth under non-limiting nitrate conditions while transgenic plants expressing AtPP1 (SEQ ID NO: 85), AtTOPP1 (SEQ ID NO: 64), AtTOPP3 (SEQ ID NO: 75), AtTOPP5 (SEQ ID NO: 65), AtTOPP6 (SEQ ID NO: 74) and AtTOPP7 (SEQ ID NO: 67, SEQ ID NO: 116 and SEQ ID NO: 118) with CaMV 35S promoter were deemed not to exhibit a root architecture phenotype different than control plants under these nitrogen conditions of 60 mM KNO₃. Transgenic plants overexpressing ZmSTPP3 (SEQ ID NO: 48) also showed enhanced root growth when grown on plates containing 4 mM KNO₃.

### Example 3

### pH Indicator Dye Assay to Identify Genes Involved in Nitrate Uptake

Analysis was performed using the following pH indicator dye assay to identify the genes involved with nitrate uptake as detailed in U.S. patent application Ser. No. 12/166,473, filed Jul. 3, 2007. Using the protocol detailed in U.S. patent application Ser. No. 12/166,473, filed Jul. 3, 2007, Arabidopsis lines overexpressing AtPP1 (SEQ ID NO: 85) with the CaMV 35S promoter had significantly less (p<0.05) nitrate remaining in the medium than wild-type controls.

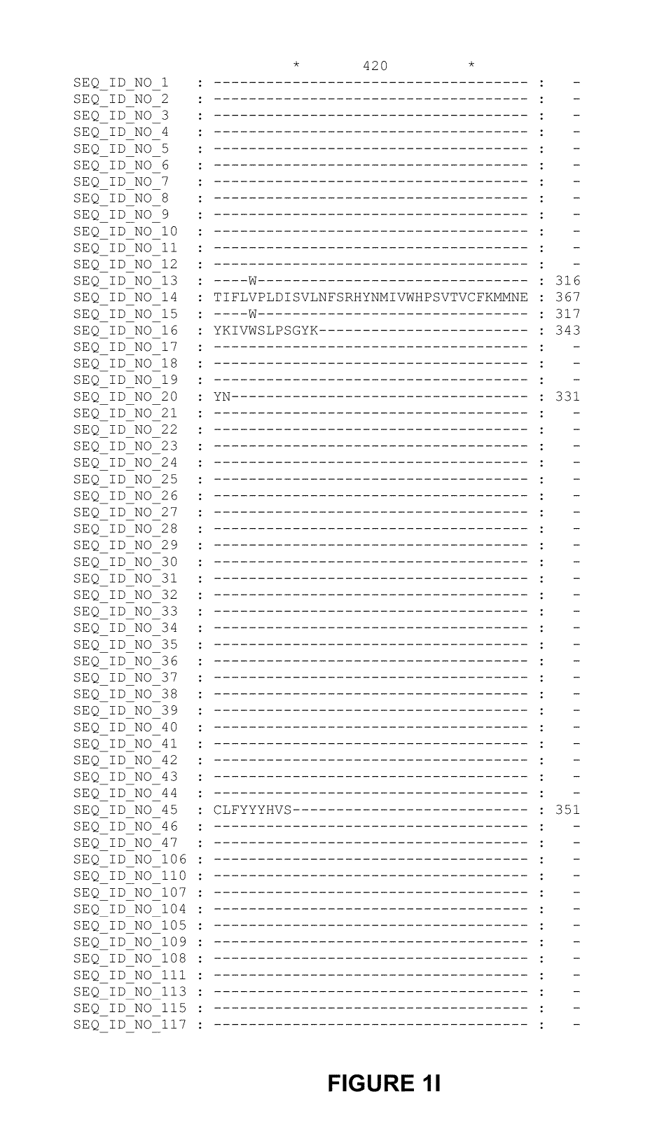

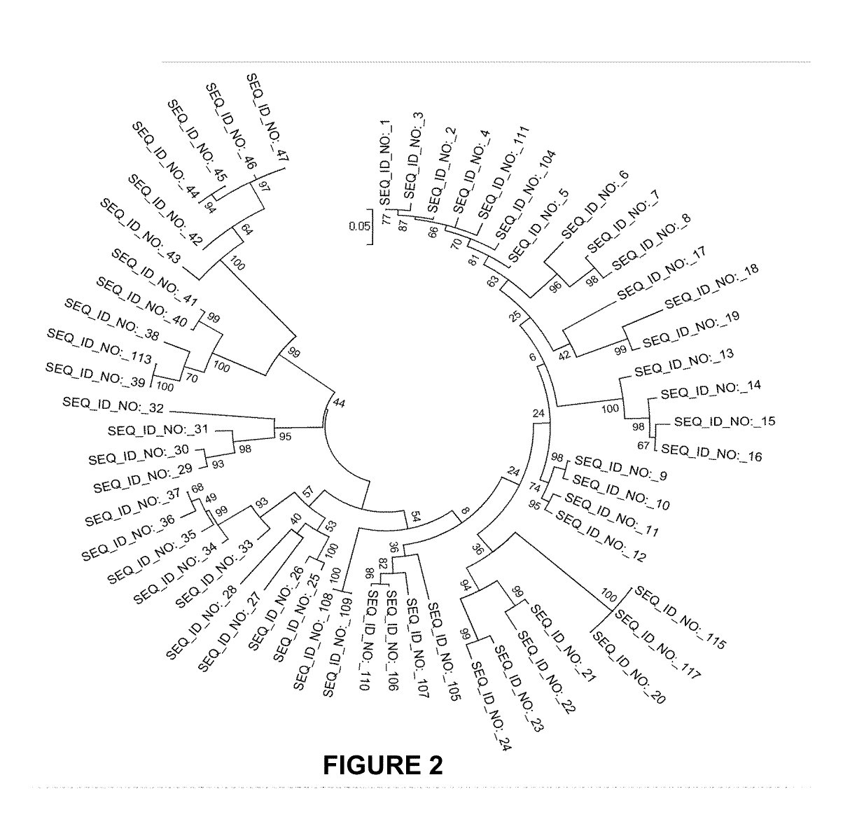

In addition to AtPP1, ZmSTPP3 (SEQ ID NO: 48) and other Arabidopsis members of the TOPP family (AtTOPP1-8; SEQ ID NO: 64, SEQ ID NO: 66, SEQ ID NO: 75, SEQ ID NO: 53, SEQ ID NO: 65, SEQ ID NO: 74, SEQ ID NO: 67, SEQ ID NO: 116, SEQ ID NO: 118, SEQ ID NO: 114, SEQ ID NO: 86) were overexpressed using the CaMV 35S promoter, transformed into Arabidopsis and analyzed in this assay. Overexpression of each of these sequences resulted in significantly less (p<0.05) nitrate remaining in the medium than wild-type controls. The Arabidopsis family members that exhibit less nitrate remaining in the medium represent each Glade from FIG. 2.

### Example 4

### Screen of Genes Under Nitrogen Limiting Conditions in Arabidopsis

Transgenic seed selected by the presence of the fluorescent marker YFP can also be screened for their tolerance to grow under nitrogen limiting conditions. Transgenic individuals expressing the Arabidopsis genes of interest are plated on Low N medium (0.5×N-Free Hoagland's, 0.4 mM potassium nitrate, 0.1% sucrose, 1 mM MES and 0.25% Phytagel™), such that 32 transgenic individuals are grown next to 32 wild-type individuals on one plate. Plants are evaluated at 10, 11, 12 and 13 days. If a line shows a statistically significant difference from the controls, the line is considered a validated nitrogen-deficiency tolerant line. After masking the plate image to remove background color, two different measurements are collected for each individual: total rosetta area and the percentage of color that falls into a green color bin. Using hue, saturation and intensity data (HIS), the green color bin consists of hues 50-66. Total rosetta area is used as a measure of plant biomass, whereas the green color bin has been shown by dose-response studies to be an indicator of nitrogen assimilation.

Transgenic plants individually overexpressing AtPP1, ZmSTPP3 and additional Arabidopsis TOPP family members were evaluated in this nitrogen limiting assay. Transgenic plants overexpressing AtPP1 (SEQ ID NO: 85), AtTOPP8-1 (SEQ ID NO: 114) or AtTOPP4 (SEQ ID NO: 53) showed an increase in total rosette area and an improvement of color in the green color bin while transgenic Arabidopsis plants expressing ZmSTPP3 (SEQ ID NO: 48), AtTOPP7-2 (SEQ ID NO: 116) or AtTOPP3 (SEQ ID NO: 75) were not considered different from control plants for rosette area but showed less color in the green color bin. AtTOPP1 (SEQ ID NO: 64). AtTOPP7-1 (SEQ ID NO: 67) showed an increase in total rosette area. In addition, transgenic plants expressing AtTOPP5 (SEQ ID NO: 65) or AtTOPP6 (SEQ ID NO: 74) with CaMV 35S promoter showed a decrease in both parameters (total rosette area and color in green color bin).

### Example 5

### Testing for Enhanced Nitrate Uptake in Arabidopsis

Candidate genes can be transformed into Arabidopsis and overexpressed under a promoter such as 35S or maize Ubiquitin promoters. If the same or similar phenotype is observed in the transgenic line as in the parent activation-tagged line, then the candidate gene is considered to be a validated “lead gene” in Arabidopsis. The Arabidopsis AtPP1 (SEQ ID NO: 85) gene can be directly tested for its ability to enhance nitrate uptake in Arabidopsis.

A 35S-At-PP1 gene construct was introduced into wild-type Arabidopsis ecotype Col-0, using standard transformation procedures.

Transgenic T2 seeds from multiple independent T1 lines may be selected by the presence of the fluorescent YFP marker. Fluorescent seeds were subjected to the pH and nitrate uptake assays following the procedures described herein. Transgenic T2 seeds were re-screened using 3 or 4 plates per construct. Each plate contained non-transformed Columbia seed to serve as a control.

### Example 6

### NUE Nitrogen: Carbon Assay

Seeds of Arabidopsis thaliana (control and transgenic line), ecotype Columbia, are surface sterilized and then plated on to 0.5×N-free Murashige and Skoog (MS) medium containing 5 mM KNO₃, 5% sucrose and 0.75% (w/v) Phytagel™ (Sigma) such that 18 wild-type and 18 transgenic seeds are on the same plate. Plates are incubated for 3 days in darkness at 4° C. to break dormancy (stratification) and transferred thereafter to growth chambers at a temperature of 22° C. under 16-hours of light and 20° C. under 8-hours of dark. The average light intensity is 140 μE/m2/s. Seedlings are grown for 14 days with the length of each leaf axis being measured at day 7 and day 10.

### Example 7

### NUE Seedling Assay Protocol

Seed of transgenic events are separated into transgene (heterozygous) and null seed using a seed color marker. Random assignments of treatments were made to each block of pots arranged using multiple replicates of all treatments. Null seeds of several events of the same construct were mixed and used as control for comparison of the positive events in this block. The transgenic parameters were compared to a bulked construct null and in the second case transgenic parameters were compared to the corresponding event null. Standard statistical analyses were used.

Two seed of each treatment were planted in 4 inch, square pots containing TURFACE®-MVP on 8 inch, staggered centers and watered four times each day with a solution containing the following nutrients:

| | | | |
|---|---|---|---|
| 1 mM CaCl₂ | 2 mM MgSO₄ | 0.5 mM KH₂PO₄ | 83 ppm Sprint330 |
| 3 mM KCl | 1 mM KNO₃ | 1 uM ZnSO₄ | 1 uM MnCl₂ |
| 3 uM H₃BO₄ | 1 uM MnCl₂ | 0.1 uM CuSO₄ | 0.1 uM NaMoO₄ |

After emergence the plants were thinned to one seed per pot. Treatments routinely were planted on a Monday, emerged the following Friday and were harvested 18 days after planting. At harvest, plants were removed from the pots and the Turface washed from the roots. The roots were separated from the shoot, placed in a paper bag and dried at 70° C. for 70 hr. The dried plant parts (roots and shoots) were weighed and placed in a 50 ml conical tube with approximately 20 5/32 inch steel balls and ground by shaking in a paint shaker. Approximately, 30 mg of the ground tissue (weight recorded for later adjustment) was hydrolyzed in 2 ml of 20% H₂O₂ and 6M H₂SO₄ for 30 min at 170° C. After cooling, water was added to 20 ml, mixed thoroughly, and a 50 μl aliquot removed and added to 950 μl 1M Na₂CO₃. The ammonia in this solution was used to estimate total reduced plant nitrogen by placing 100 μl of this solution in individual wells of a 96 well plate followed by adding 50 μl of OPA solution. Fluorescence, excitation=360 nM/emission=530 nM, was determined and compared to NH₄Cl standards dissolved in a similar solution and treated with OPA solution.

OPA solution—5 ul Mercaptoethanol+1 ml OPA stock solution (make fresh, daily)

OPA stock—50 mg o-phthadialdehyde (OPA-Sigma #P0657) dissolved in 1.5 ml methanol+4.4 ml 1M Borate buffer pH9.5 (3.09 g H₃BO₄+1 g NaOH in 50 ml water)+0.55 ml 20% SDS(make fresh weekly)

Using these data the following parameters were measured and means compared to null mean parameters using a Student's t test:

Total Plant Biomass

Root Biomass

Shoot Biomass

Root/Shoot Ratio

Plant N concentration

Total Plant N

Variance was calculated within each block using a nearest neighbor calculation as well as by Analysis of Variance (ANOV) using a completely random design (CRD) model. An overall treatment effect for each block was calculated using an F statistic by dividing overall block treatment mean square by the overall block error mean square.

### Example 8

### Inter-Relationship of Related Proteins

Phylogenetic and Motif Analyses for PP1 Genes in Arabidopsis thaliana, Zea mays, Oryza sativa, Sorghum bicolor, Glycine max, Pennisetum glaucum, Dennstaedtia punctilobula and Paspalum notatum

Serine/Threonine-specific phosphoprotein phosphatase (STPP) represents a large family of phosphatases that dephosphorylate Ser/Thr side chains. This greater STPP family includes PP1, PP2A and other subfamilies. Protein sequences are highly conserved within each STPP subfamily. The activity, specificity, and localization of STPP catalytic subunits are largely determined by their interacting regulatory subunits. The following analysis focuses on the PP1 subfamily of STPP proteins.

STPP related gene homologs in Maize, Soybean, Sorghum, Rice, Fern, Pearl millet and Bahia grass were collected for Arabidopsis (TAIR10) PP1-like proteins. A total of 58 homologs with at least 70% identify and 80% coverage to PP1 proteins are found in all the other seven plant species. These sequences are highly similar to each other and share a common Pfam domain Metallophos (PF00149). All 58 PP1 sequences are listed in Table 1 in further detail. A phylogenetic tree (FIG. 2) was constructed for the 58 PP1 sequences using MEGA5 software. The PP1 sequences are further grouped into different clusters with respect to key branch points in the dendrogram.

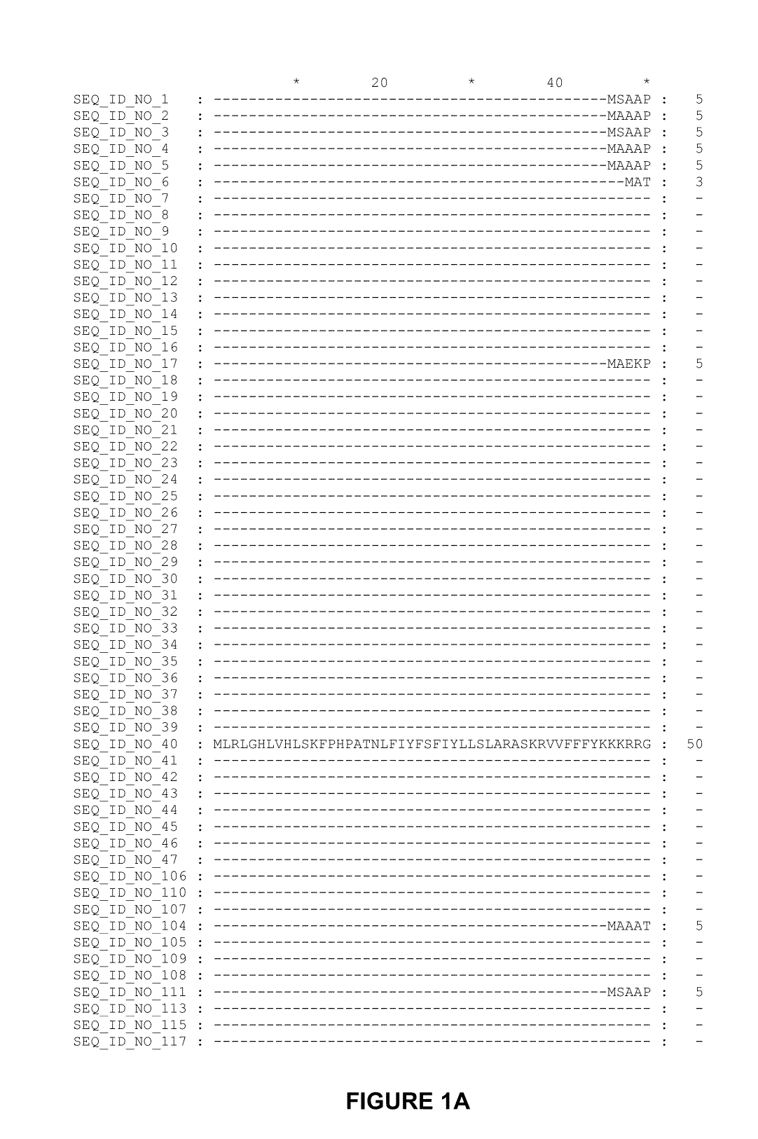

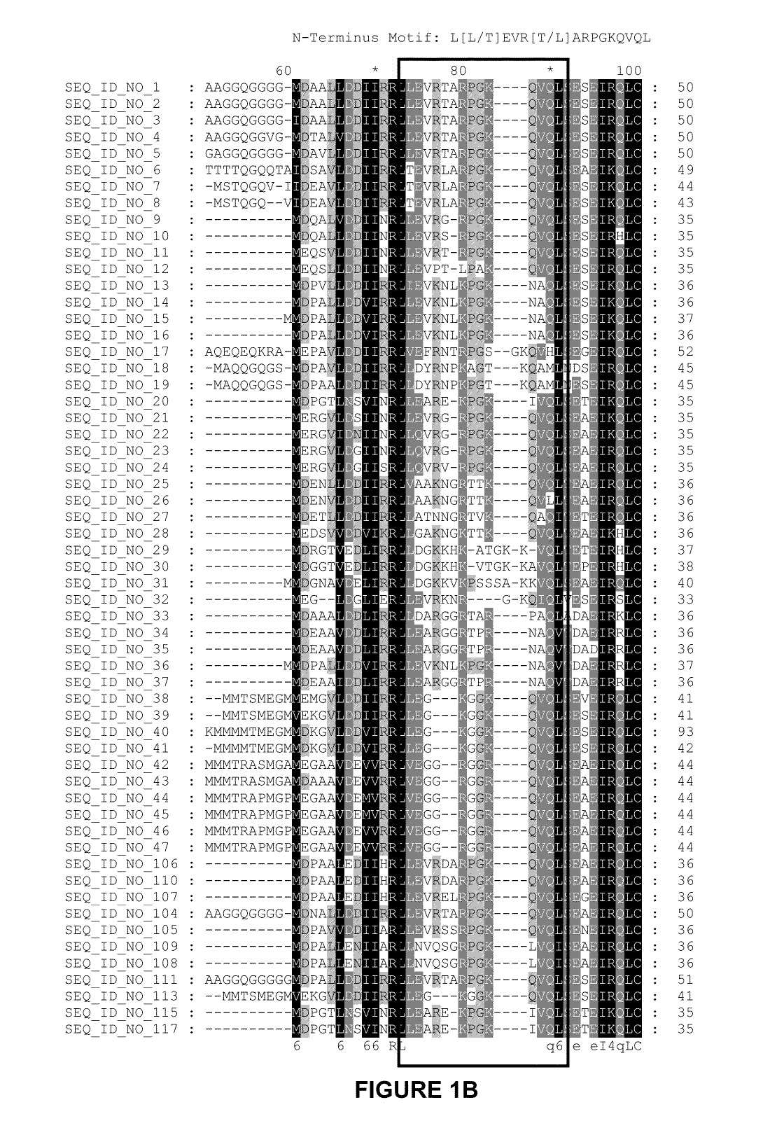

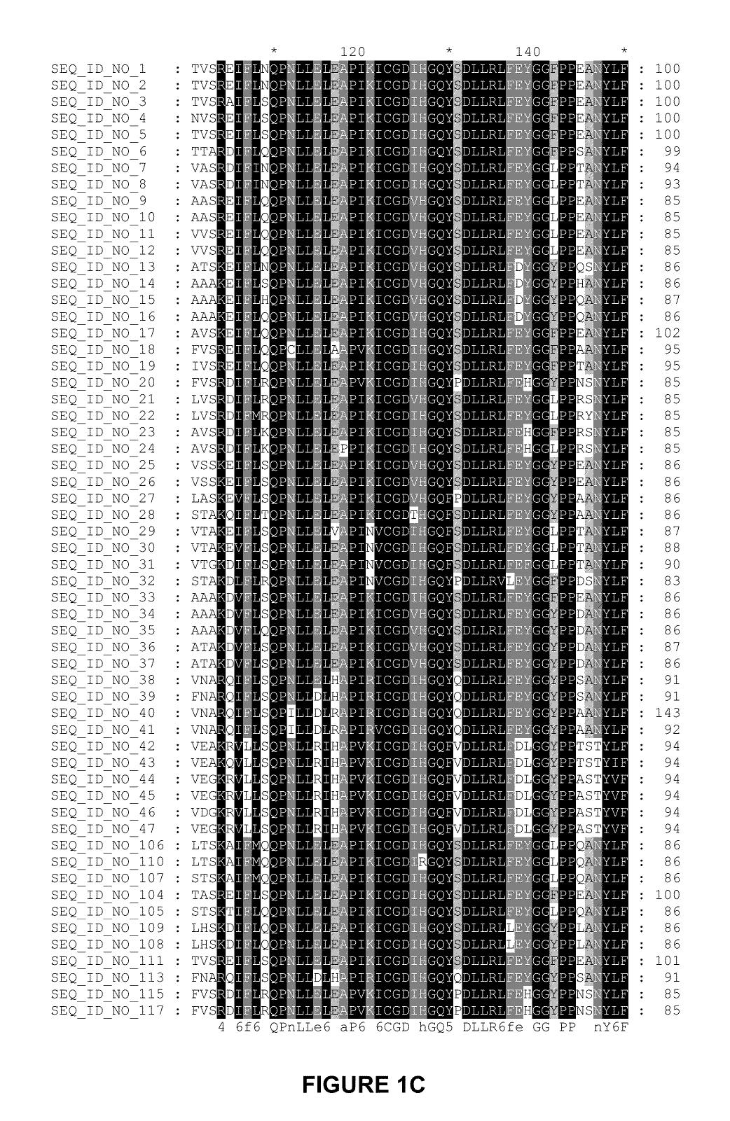

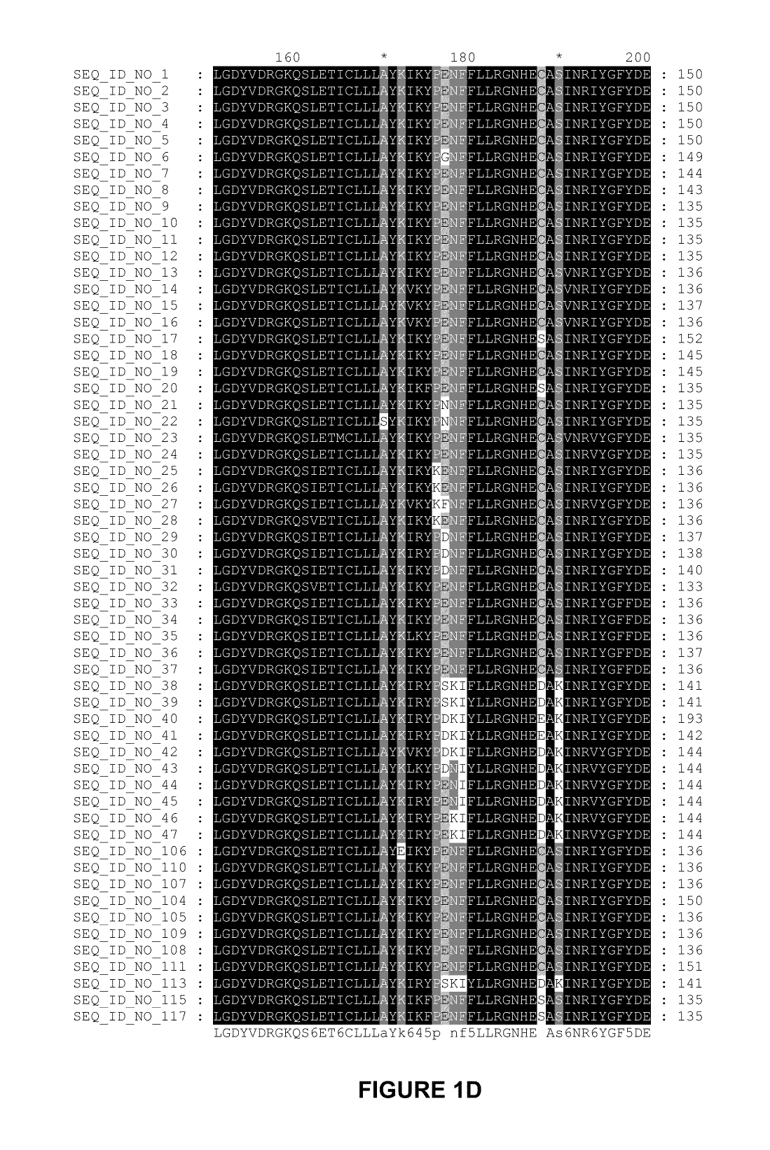

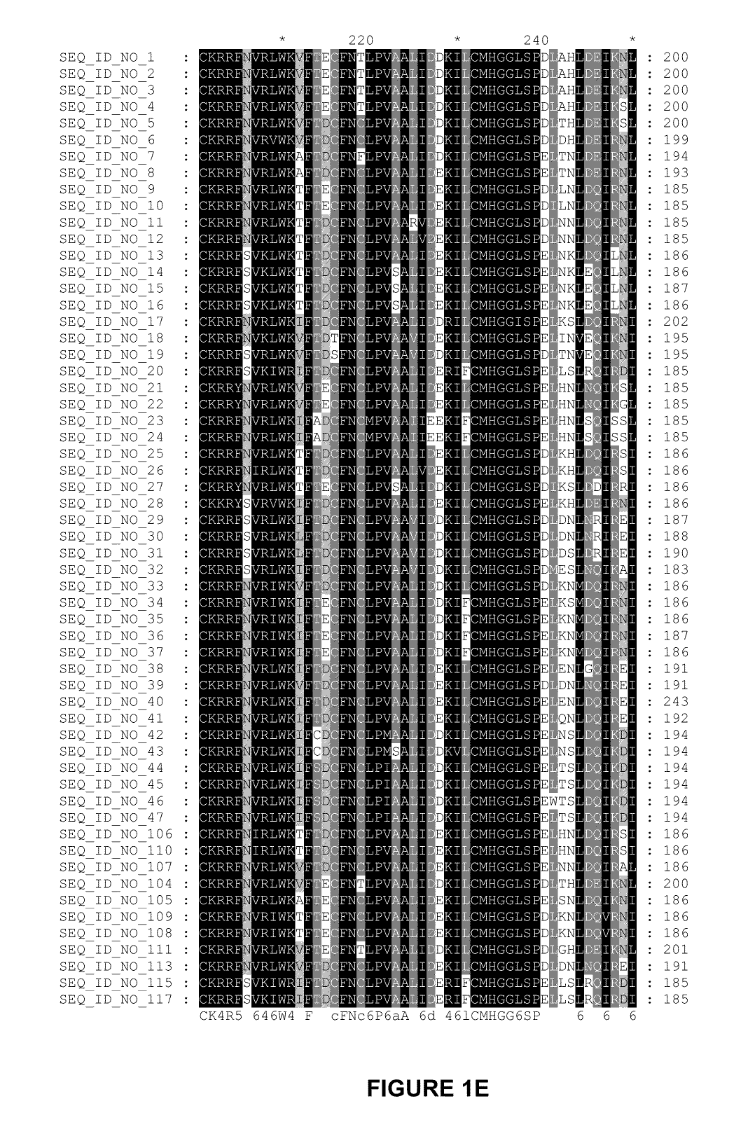

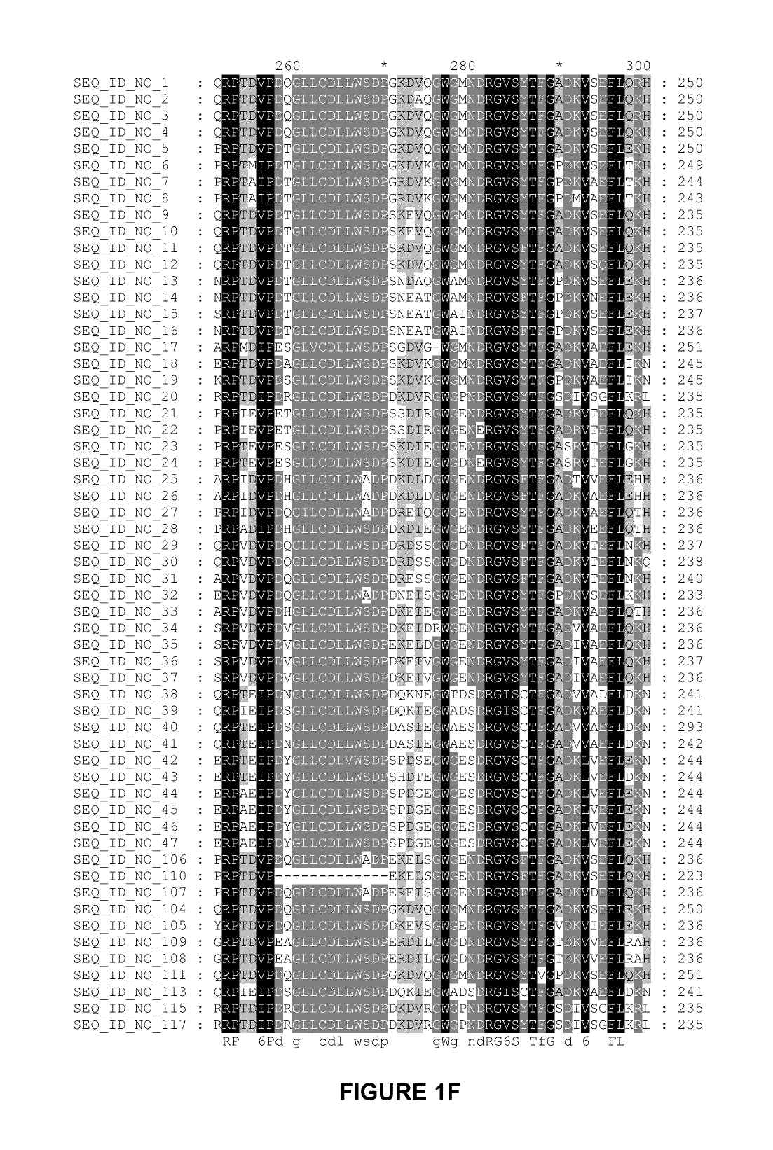

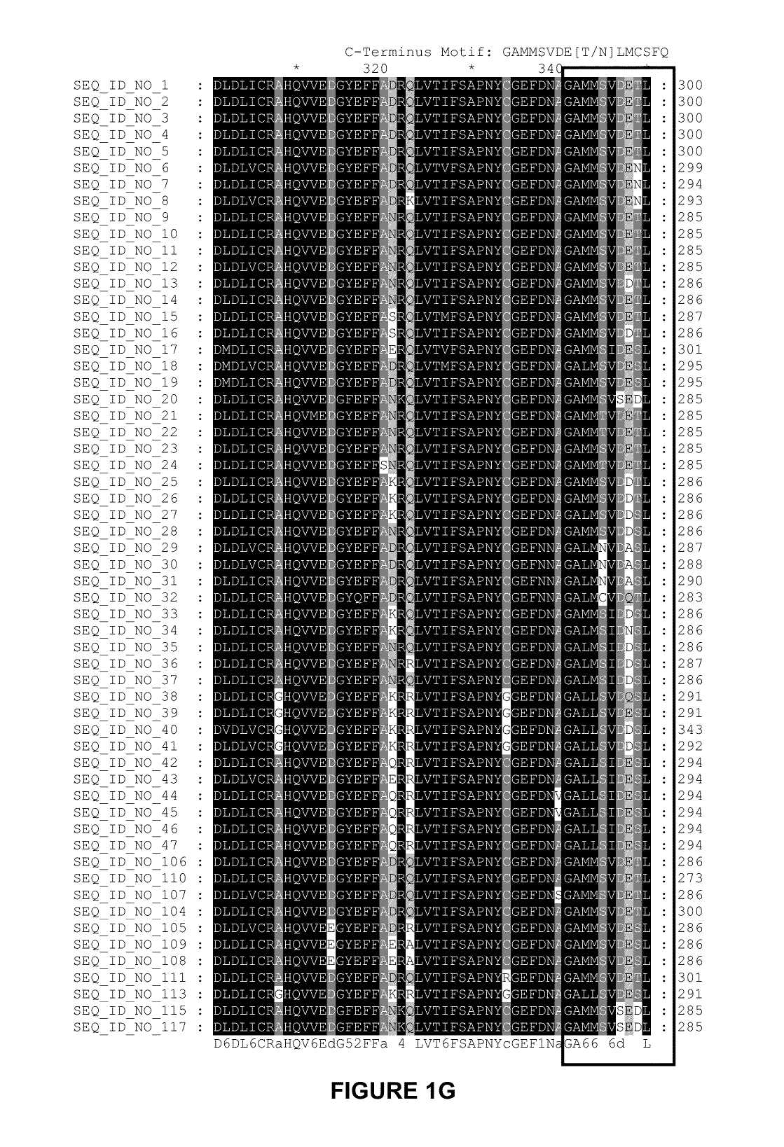

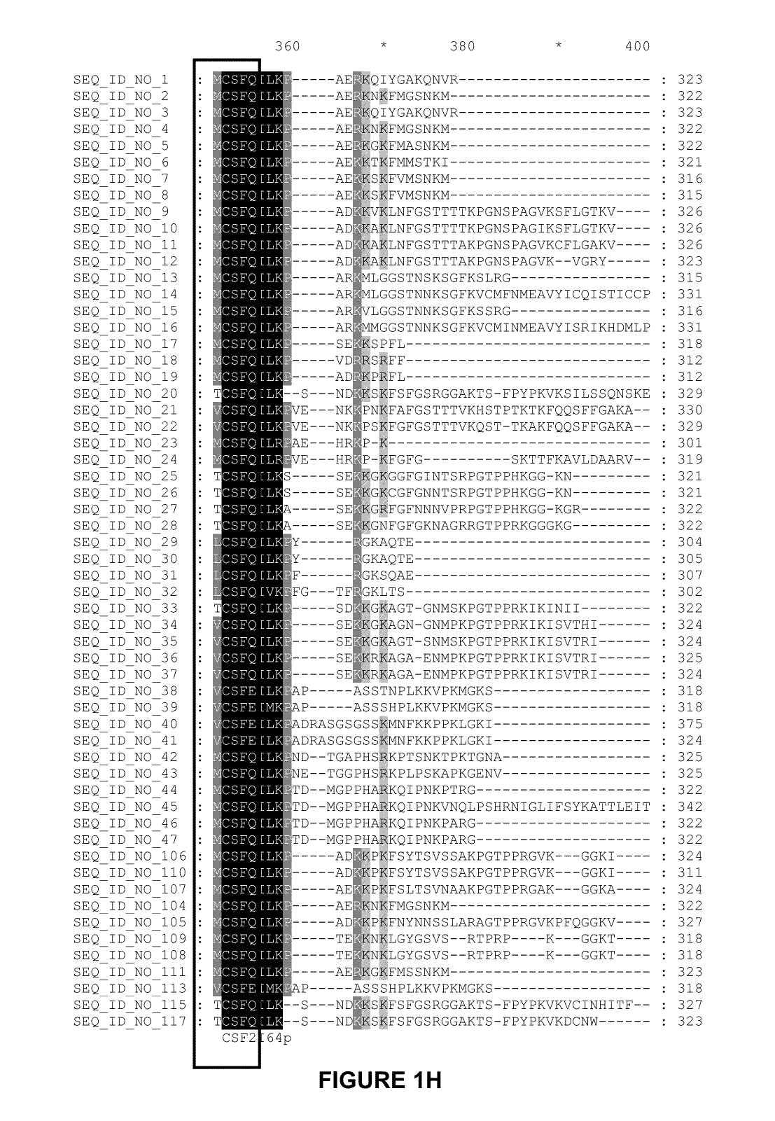

Pfam domain analysis showed that the central region (approximately from amino acids 69 to 261) contains a conserved Metallophos domain for the PP1 proteins that were analyzed. The functional relationship including any difference for genes within PP1 subfamily is likely caused by the differences in the C and N terminus. The motif analysis was conducted to identify conserved N and C terminus motifs for STPP3 proteins using MEME (Multiple EM for Motif Elicitation) and ClustalX tools. A N-terminus motif L[L/T]EVR[T/L]ARPGKQVQL and a C-terminus motif GAMMSVDE[T/N]LMCSFQ are identified for STPP3 proteins. These motifs are indicated in the multiple sequence alignment profile in the FIG. 1. These motifs likely play a functional role for STPP3 by interacting with regulatory subunits

### Example 9

### Composition of cDNA Libraries; Isolation and Sequencing of cDNA Clones

cDNA libraries representing mRNAs from various tissues of Canna edulis (Canna), Momordica charantia (balsam pear), Brassica (mustard), Cyamopsis tetragonoloba (guar), Zea mays (maize), Otyza sativa (rice), Glycine max (soybean), Helianthus annuus (sunflower) and Triticum aestivum (wheat) were prepared. cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.).

Full-insert sequence (FIS) data is generated utilizing a modified transposition protocol. Clones identified for FIS are recovered from archived glycerol stocks as single colonies and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke, (1994) Nucleic Acids Res. 22:3765-3772). The in vitro transposition system places unique binding sites randomly throughout a population of large DNA molecules. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred and Phrap (Ewing, et al., (1998) Genome Res. 8:175-185; Ewing and Green, (1998) Genome Res. 8:186-194). The resulting DNA fragment is ligated into a pBluescript vector using a commercial kit and following the manufacturer's protocol. This kit is selected from many available from several vendors including Invitrogen™ (Carlsbad, Calif.), Promega Biotech (Madison, Wis.), and Gibco-BRL (Gaithersburg, Md.). The plasmid DNA is isolated by alkaline lysis method and submitted for sequencing and assembly using Phred/Phrap, as above.

### Example 10

### Identification of cDNA Clones

cDNA clones encoding nitrate uptake-associated-like polypeptides were identified by conducting BLAST (Basic Local Alignment Search Tool; Altschul, et al., (1993) J. Mol. Biol. 215:403-410; see also, the explanation of the BLAST algorithm on the world wide web site for the National Center for Biotechnology Information at the National Library of Medicine of the National Institutes of Health) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL and DDBJ databases). The cDNA sequences obtained as described herein were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States, (1993) Nat. Genet. 3:266-272) provided by the NCBI. For convenience, the P-value (probability) of observing a match of a cDNA sequence to a sequence contained in the searched databases merely by chance as calculated by BLAST are reported herein as “p Log” values, which represent the negative of the logarithm of the reported P-value. Accordingly, the greater the p Log value, the greater the likelihood that the cDNA sequence and the BLAST “hit” represent homologous proteins.

ESTs submitted for analysis are compared to the Genbank database as described above. ESTs that contain sequences more 5- or 3-prime can be found by using the BLASTn algorithm (Altschul, et al., (1997) Nucleic Acids Res. 25:3389-3402.) against nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described herein. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 11

### Preparation of a Plant Expression Vector

A PCR product obtained using methods that are known by one skilled in the art can be combined with the Gateway® donor vector, such as pDONR™/Zeo (Invitrogen™). Using the Invitrogen™ Gateway® Clonase™ technology, the homologous At3g05580 gene from the entry clone can then be transferred to a suitable destination vector to obtain a plant expression vector for use with Arabidopsis and corn. For example, an expression vector contains At3g05580 expressed by the maize ubiquitin promoter, a herbicide resistance cassette and a seed sorting cassette.

### Example 12

### Agrobacterium Mediated Transformation into Maize

Maize plants can be transformed to overexpress a validated Arabidopsis lead gene or the corresponding homologs from various species in order to examine the resulting phenotype.

Agrobacterium-mediated transformation of maize is performed essentially as described by Zhao, et al., (2006) Meth. Mol. Biol. 318:315-323 (see also, Zhao, et al., (2001) Mol. Breed. 8:323-333 and U.S. Pat. No. 5,981,840, issued Nov. 9, 1999, incorporated herein by reference). The transformation process involves bacterium innoculation, co-cultivation, resting, selection and plant regeneration.

Phenotypic analysis of transgenic T0 plants and T1 plants can be performed.

T1 plants can be analyzed for phenotypic changes. Using image analysis T1 plants can be analyzed for phenotypical changes in plant area, volume, growth rate and color analysis can be taken at multiple times during growth of the plants. Alteration in root architecture can be assayed as described herein.

Subsequent analysis of alterations in agronomic characteristics can be done to determine whether plants containing the validated Arabidopsis lead gene have an improvement of at least one agronomic characteristic, when compared to the control (or reference) plants that do not contain the validated Arabidopsis lead gene. The alterations may also be studied under various environmental conditions.

### Example 13

### Transformation of Maize with Validated Arabidopsis Lead Genes Using Particle Bombardment

Maize plants can be transformed to overexpress a validated Arabidopsis lead gene or the corresponding homologs from various species in order to examine the resulting phenotype.

The Gateway® entry clones described in Example 12 can be used to directionally clone each gene into a maize transformation vector. Expression of the gene in maize can be under control of a constitutive promoter such as the maize ubiquitin promoter (Christensen, et al., (1989) Plant Mol. Biol. 12:619-632 and Christensen, et al., (1992) Plant Mol. Biol. 18:675-689)

The recombinant DNA construct described above can then be introduced into maize cells by the following procedure. Immature maize embryos can be dissected from developing caryopses derived from crosses of the inbred maize lines H99 and LH132. The embryos are isolated ten to eleven days after pollination when they are 1.0 to 1.5 mm long. The embryos are then placed with the axis-side facing down and in contact with agarose-solidified N6 medium (Chu, et al., (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every two to three weeks.

The particle bombardment method (Klein, et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After ten minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the maize tissue with a Biolistic @ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains bialaphos (5 mg per liter) and lacks casein or proline. The tissue continues to grow slowly on this medium. After an additional two weeks the tissue can be transferred to fresh N6 medium containing bialaphos. After six weeks, areas of about 1 cm in diameter of actively growing callus can be identified on some of the plates containing the bialaphos-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839). Transgenic T0 plants can be regenerated and their phenotype determined following HTP procedures. T1 seed can be collected.

T1 plants can be grown and analyzed for phenotypic changes. The following parameters can be quantified using image analysis: plant area, volume, growth rate and color analysis can be collected and quantified. Expression constructs that result in an alteration of root architecture or any one of the agronomic characteristics listed above compared to suitable control plants, can be considered evidence that the Arabidopsis lead gene functions in maize to alter root architecture or plant architecture.

Furthermore, a recombinant DNA construct containing a validated Arabidopsis gene can be introduced into a maize line either by direct transformation or introgression from a separately transformed line.

Transgenic plants, either inbred or hybrid, can undergo more vigorous field-based experiments to study root or plant architecture, yield enhancement and/or resistance to root lodging under various environmental conditions (e.g., variations in nutrient and water availability).

Subsequent yield analysis can also be done to determine whether plants that contain the validated Arabidopsis lead gene have an improvement in yield performance, when compared to the control (or reference) plants that do not contain the validated Arabidopsis lead gene. Plants containing the validated Arabidopsis lead gene would improved yield relative to the control plants, preferably 50% less yield loss under adverse environmental conditions or would have increased yield relative to the control plants under varying environmental conditions.

### Example 14

### Electroporation of Agrobacterium tumefaciens LBA4404

Electroporation competent cells (40 μl), such as Agrobacterium tumefaciens LBA4404 (containing PHP10523), are thawn on ice (20-30 min). PHP10523 contains VIR genes for T-DNA transfer, an Agrobacterium low copy number plasmid origin of replication, a tetracycline resistance gene and a cos site for in vivo DNA biomolecular recombination. Meanwhile the electroporation cuvette is chilled on ice. The electroporator settings are adjusted to 2.1 kV.

A DNA aliquot (0.5 μL JT (U.S. Pat. No. 7,087,812) parental DNA at a concentration of 0.2 μg-1.0 μg in low salt buffer or twice distilled H₂O) is mixed with the thawn Agrobacterium cells while still on ice. The mix is transferred to the bottom of electroporation cuvette and kept at rest on ice for 1-2 min. The cells are electroporated (Eppendorf electroporator 2510) by pushing “Pulse” button twice (ideally achieving a 4.0 msec pulse). Subsequently 0.5 ml 2×YT medium (or SOCmedium) are added to cuvette and transferred to a 15 ml Falcon tube. The cells are incubated at 28-30° C., 200-250 rpm for 3 h.

Aliquots of 250 μl are spread onto #30B (YM+50 μg/mL Spectinomycin) plates and incubated 3 days at 28-30° C. To increase the number of transformants one of two optional steps can be performed:

### Option 1:

overlay plates with 30 μl of 15 mg/ml Rifampicin. LBA4404 has a chromosomal resistance gene for Rifampicin. This additional selection eliminates some contaminating colonies observed when using poorer preparations of LBA4404 competent cells.

### Option 2:

Perform two replicates of the electroporation to compensate for poorer electrocompetent cells.

### Identification of Transformants:

Four independent colonies are picked and streaked on AB minimal medium plus 50 mg/mL Spectinomycin plates (#12S medium) for isolation of single colonies. The plated are incubate at 28° C. for 2-3 days.

A single colony for each putative co-integrate is picked and inoculated with 4 ml #60A with 50 mg/l Spectinomycin. The mix is incubated for 24 h at 28° C. with shaking. Plasmid DNA from 4 ml of culture is isolated using Qiagen Miniprep+optional PB wash. The DNA is eluted in 30 μl. Aliquots of 2 μl are used to electroporate 20 μl of DH10b+20 μl of ddH₂O as per above.

Optionally a 15 μl aliquot can be used to transform 75-100 μl of Invitrogen™-Library Efficiency DH5α. The cells are spread on LB medium plus 50 mg/mL Spectinomycin plates (#34T medium) and incubated at 37° C. overnight.

Three to four independent colonies are picked for each putative co-integrate and inoculated 4 ml of 2×YT (#60A) with 50 μg/ml Spectinomycin. The cells are incubated at 37° C. overnight with shaking.

The plasmid DNA is isolated from 4 ml of culture using QIAprep® Miniprep with optional PB wash (elute in 50 μl) and 8 μl are used for digestion with SalI (using JT parent and PHP10523 as controls).

Three more digestions using restriction enzymes BamHI, EcoRI and HindIII are performed for 4 plasmids that represent 2 putative co-integrates with correct SalI digestion pattern (using parental DNA and PHP10523 as controls). Electronic gels are recommended for comparison.

### Example 15

### Transformation of Gaspe Bay Flint Derived Maize Lines with Validated Arabidopsis Lead Genes and Corresponding Homologs from Other Species

Maize plants can be transformed as described in Example 13-15 overexpressing ZmSTPP3 (SEQ ID NO: 48) gene and the corresponding homologs from other species, such as the ones listed in Table 1 in order to examine the resulting phenotype. Promoters including but not limited to the maize Ubiquitin promoter, the S2A promoter, the maize ROOTMET2 promoter, the maize Cyclo, the CR1BIO, the CRWAQ81 and others are useful for directing expression of homologs of ZmSTPP3 in maize. Furthermore, a variety of terminators, such as, but not limited to the PINII terminator, can be used to achieve expression of the gene of interest in Gaspe Bay Flint Derived Maize Lines.

Recipient Plants

Recipient plant cells can be from a uniform maize line having a short life cycle (“fast cycling”), a reduced size and high transformation potential. Typical of these plant cells for maize are plant cells from any of the publicly available Gaspe Bay Flint (GBF) line varieties. One possible candidate plant line variety is the F1 hybrid of GBF×QTM (Quick Turnaround Maize, a publicly available form of Gaspe Bay Flint selected for growth under greenhouse conditions) disclosed in Tomes, et al., US Patent Application Publication Number 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line) X Gaspe Flint. Yet another suitable line is a transformable elite inbred line carrying a transgene which causes early flowering, reduced stature or both.

Transformation Protocol

Any suitable method may be used to introduce the transgenes into the maize cells, including but not limited to inoculation type procedures using Agrobacterium based vectors as described in Examples 13 and 14. Transformation may be performed on immature embryos of the recipient (target) plant.

Plant Growth and Identification

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a., a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified throughout the evaluation process and the data gathered from that plant is associated with that plant so that the gathered data can be associated with the transgene carried by the plant.

Phenotypic Analysis Using Three-Dimensional Imaging

Each greenhouse plant in the T0 event population, including any control plants, is analyzed for agronomic characteristics of interest and the agronomic data for each plant is recorded or stored in a manner so that it is associated with the identifying data (see above) for that plant. Confirmation of a phenotype (gene effect) can be accomplished in the T1 generation with a similar experimental design to that described above. The T0 plants are analyzed at the phenotypic level using quantitative, non-destructive imaging technology throughout the plant's entire greenhouse life cycle to assess the traits of interest. Any suitable imaging instrumentation may be used.

Software

The imaging analysis system comprises a software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems can be used for quantitative interpretation of the imaging data and any of these software systems can be applied to the image data set.

Illumination

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging

For best estimation of biomass the plant images are taken from three axes, preferably the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2Area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification

The imaging technology may also be used to determine plant color and to assign plant colors to various color classes. The assignment of image colors to color classes is a feature of the software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins) and that other color classification schemes can provide further measures of gene action in traits related to these responses.

Plant Architecture Analysis

Transgenes which modify plant architecture parameters may also be identified, including such parameters as maximum height and width, internodal distances, angle between leaves and stem, number of leaves starting at nodes and leaf length. The software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the light spectrum digital analyzing device. A computer with a barcode reader, a palm device or a notebook PC may be used for ease of data capture recording time of observation, plant identifier and the operator who captured the data.

Orientation of the Plants

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant.

### Example 15

### Screening of Gaspe Bay Flint Derived Maize Lines Under Nitrogen Limiting Conditions

Transgenic plants will contain two or three doses of Gaspe Flint-3 with one dose of GS3 (GS3/(Gaspe-3)2X or GS3/(Gaspe-3)3X) and will segregate 1:1 for a dominant transgene. Plants will be planted in TURFACE®, a commercial potting medium, and watered four times each day with 1 mM KNO₃ growth medium and with 2 mM KNO₃ or higher, growth medium. Control plants grown in 1 mM KNO₃ medium will be less green, produce less biomass and have a smaller ear at anthesis. Statistical analysis is used to decide if differences seen between treatments are different.

Expression of a transgene will result in plants with improved plant growth in 1 mM KNO₃ when compared to a transgenic null. Thus biomass and greenness are monitored during growth and compared to a transgenic null. Improvements in growth, greenness and ear size at anthesis will be indications of increased nitrogen tolerance.

### Example 16

### Transgenic Maize Plants

T₀ transgenic maize plants containing the nitrate uptake-associated construct under the control of a promoter were generated. These plants were grown in greenhouse conditions for Gaspe-derived corn plants, for example, as described in US Patent Application Publication Number 2003/0221212, U.S. patent application Ser. No. 10/367,417.

Each of the plants was analyzed for measurable alteration in one or more of the following characteristics in the following manner:

T₁ progeny derived from self fertilization each T₀ plant containing a single copy of each nitrate uptake-associated construct that were found to segregate 1:1 for the transgenic event were analyzed for improved growth rate in suboptimal KNO₃. Growth was monitored up to anthesis when cumulative plant growth, growth rate and ear weight were determined for transgene positive, transgene null and non-transformed controls events. The distribution of the phenotype of individual plants was compared to the distribution of a control set and to the distribution of all the remaining treatments. Variances for each set were calculated and compared using an F test, comparing the event variance to a non-transgenic control set variance and to the pooled variance of the remaining events in the experiment. The greater the response to KNO₃, the greater the variance within an event set and the greater the F value. Positive results will be compared to the distribution of the transgene within the event to make sure the response segregates with the transgene.

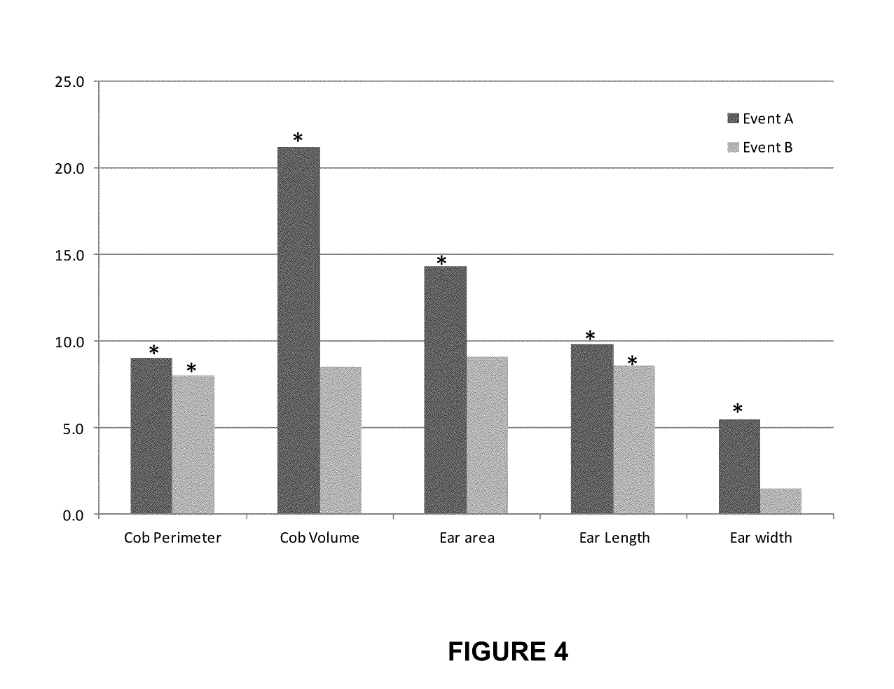

Transgenic expression of ZmSTPP3 with corn UBI promoter enhances ear growth and development in the greenhouse NUE reproductive assay, in which the plants are subjected to suboptimal nitrogen treatment from planting to harvesting. As shown in FIG. 4, two events were found to have significantly increased cob perimeter by 9.0% and 8.0% and ear length by 9.8% and 8.6% over non transgenic controls, respectively (p<0.1). In addition, the cob volume, ear area and ear width of Event A are all significantly increased by 21.2%, 14.3% and 5.5% (p<0.1) comparing with the controls, respectively.

### Example 17

### Maize Transgenic Analysis from Field Plots

Transgenic events were molecularly characterized for transgene copy number and expression by PCR. Events containing single copy of transgene with detectable transgene expression were advanced for field testing. Test cross/hybrid seeds were produced and tested in field in multi-years/locations/replications experiments both in normal and low N fields. Transgenic events were evaluated in field plots where yield is limited by reducing fertilizer application by 30% or more. Statistically significant improvements in yield, yield components or other agronomic traits between transgenic and non-transgenic plants in these reduced or normal nitrogen fertility plots were used to assess the efficacy of transgene expression. The constructs with multiple events showing significant improvements (when compared to nulls) in yield or its components in multiple locations were advanced for further testing.

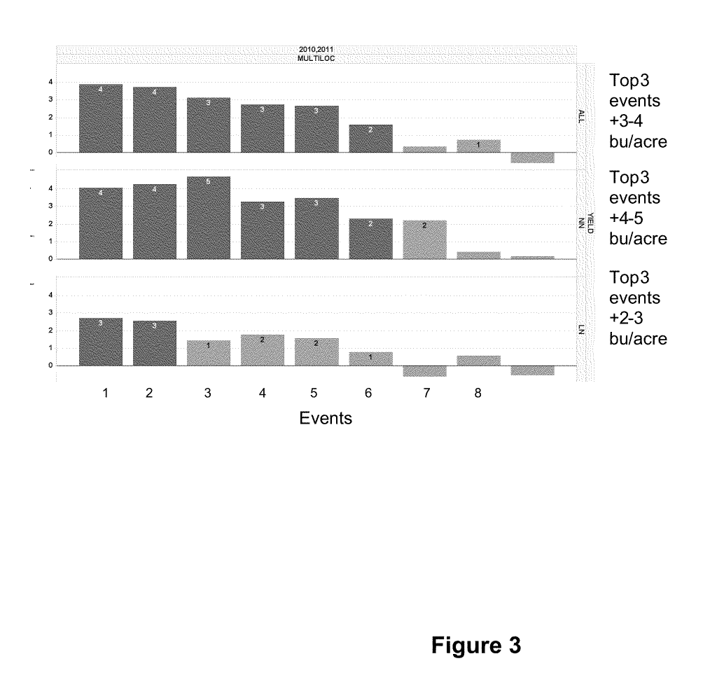

In addition to At3g05580, three maize homologs were also evaluated in field plots. According to Table 1, At3g05580 is a member of serine threonine protein phosphatase (STPP) cluster 3.1, and the three maize homologs represent three different STPP clusters. STPP1 (SEQ ID NO: 44) is a member of cluster 3.2 while STPP2 (SEQ ID NO: 29) is a member of cluster 2.2, with STPP3 (SEQ ID NO: 1) being a member of cluster 1.1. Multiple transgenic events overexpressing maize homolog STPP1 with a constitutive promoter resulted in a significant yield decrease under both nitrogen conditions. Under nitrogen-limiting conditions multiple events overexpressing maize homolog STPP2 showed a significant yield decrease while multiple events showed a significant yield increase under normal nitrogen conditions. Multiple transgenic events overexpressing the maize homolog STPP3 with a constitutive promoter showed a significant yield increase under normal and low N conditions nitrogen conditions in multiple-testers/years/locations (FIG. 3). Top 3 events showed an increase of 2-3 bu/acre and 4-5 bu/acre in low and normal N conditions, respectively (FIG. 3). In combined analyses of yield data from low and normal N depicted an increase of 3-4 bu/acre in top 3 events (FIG. 3). Transgenic events may have different expression levels of the transgene or different protein levels. STPP3 contains the N-terminus motif L[L/T]EVR[T/L]ARPGKQVQL and the C-terminus motif GAMMSVDE[T/N]LMCSFQ while STPP1 does not contain these motifs.

### Example 18

### Soybean Embryo Transformation

Soybean embryos are bombarded with a plasmid containing an antisense nitrate uptake-associated sequences operably linked to an ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

### Example 19

### Sunflower Meristem Tissue Transformation

Sunflower meristem tissues are transformed. Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water. Sunflower meristem based transformation is known in the art.

### Example 20

### Rice Tissue Transformation

### Genetic Confirmation of the Nitrate Uptake-Associated Gene

One method for transforming DNA into cells of higher plants that is available to those skilled in the art is high-velocity ballistic bombardment using metal particles coated with the nucleic acid constructs of interest (see, Klein, et al., (1987) Nature (London) 327:70-73 and see, U.S. Pat. No. 4,945,050). A Biolistic PDS-1000/He (BioRAD Laboratories, Hercules, Calif.) is used for these complementation experiments. The particle bombardment technique is used to transform the nitrate uptake-associated mutants and wild type rice with DNA fragments.

The bacterial hygromycin B phosphotransferase (Hpt II) gene from Streptomyces hygroscopicus that confers resistance to the antibiotic is used as the selectable marker for rice transformation. In the vector, pML18, the Hpt II gene was engineered with the 35S promoter from Cauliflower Mosaic Virus and the termination and polyadenylation signals from the octopine synthase gene of Agrobacterium tumefaciens. pML18 was described in WO 1997/47731, which was published on Dec. 18, 1997, the disclosure of which is hereby incorporated by reference.

Embryogenic callus cultures derived from the scutellum of germinating rice seeds serve as source material for transformation experiments. This material is generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos is the transferred to CM media (N6 salts, Nitsch and Nitsch vitamins, 1 mg/l 2,4-D, Chu, et al., 1985, Sci. Sinica 18: 659-668). Callus cultures are maintained on CM by routine sub-culture at two week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr in the dark. The petri dish lids are then left ajar for 20-45 minutes in a sterile hood to allow moisture on tissue to dissipate.

Each genomic DNA fragment is co-precipitated with pML18 containing the selectable marker for rice transformation onto the surface of gold particles. To accomplish this, a total of 10 μg of DNA at a 2:1 ratio of trait:selectable marker DNAs are added to 50 μl aliquot of gold particles that have been resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are then washed twice with 1 ml of absolute ethanol and then resuspended in 50 μl of absolute ethanol and sonicated (bath sonicator) for one second to disperse the gold particles. The gold suspension is incubated at −70° C. for five minutes and sonicated (bath sonicator) if needed to disperse the particles. Six μl of the DNA-coated gold particles are then loaded onto mylar macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Two to four plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates are incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% gelrite+50 ppm hyg B) for 2 weeks in the dark at 25° C. After 2 weeks the callus is transferred to RM2 media (MS salts, Nitsch and Nitsch vitamins, 3% sucrose, 0.4% gelrite+50 ppm hyg B) and placed under cool white light (˜40 μm⁻²s⁻¹) with a 12 hr photo period at 25° C. and 30-40% humidity. After 2-4 weeks in the light, callus begin to organize and form shoots. Shoots are removed from surrounding callus/media and gently transferred to RM3 media (½×MS salts, Nitsch and Nitsch vitamins, 1% sucrose+50 ppm hygromycin B) in phytatrays (Sigma Chemical Co., St. Louis, Mo.) and incubation is continued using the same conditions as described in the previous step.

Plants are transferred from RM3 to 4″ pots containing Metro mix 350 after 2-3 weeks, when sufficient root and shoot growth have occurred. The seed obtained from the transgenic plants is examined for genetic complementation of the nitrate uptake-associated mutation with the wild-type genomic DNA containing the nitrate uptake-associated gene.

### Example 21

### Assays to Determine Alterations of Root Architecture in Maize

Transgenic maize plants are assayed for changes in root architecture at seedling stage, flowering time or maturity. Assays to measure alterations of root architecture of maize plants include, but are not limited to the methods outlined below. To facilitate manual or automated assays of root architecture alterations, corn plants can be grown in clear pots.
1) Root mass (dry weights). Plants are grown in Turface®, a growth medium that allows easy separation of roots. Oven-dried shoot and root tissues are weighed and a root/shoot ratio calculated.
2) Levels of lateral root branching. The extent of lateral root branching (e.g., lateral root number, lateral root length) is determined by sub-sampling a complete root system, imaging with a flat-bed scanner or a digital camera and analyzing with WinRHIZO™ software (Regent Instruments Inc.).
3) Root band width measurements. The root band is the band or mass of roots that forms at the bottom of greenhouse pots as the plants mature. The thickness of the root band is measured in mm at maturity as a rough estimate of root mass.
4) Nodal root count. The number of crown roots coming off the upper nodes can be determined after separating the root from the support medium (e.g., potting mix). In addition the angle of crown roots and/or brace roots can be measured. Digital analysis of the nodal roots and amount of branching of nodal roots form another extension to the aforementioned manual method.

All data taken on root phenotype are subjected to statistical analysis, normally a t-test to compare the transgenic roots with those of non-transgenic sibling plants. One-way ANOVA may also be used in cases where multiple events and/or constructs are involved in the analysis.

### Example 22

### Variants of Disclosed Sequences

Additional sequences can be generated by known means including but not limited to truncations and point mutationa. These variants can be assessed for their impact on male fertility by using standard transformation, regeneration and evaluation protocols.

A. Variant Nucleotide Sequences that do not Alter the Encoded Amino Acid Sequence

The disclosed nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants is altered, the amino acid sequence encoded by the open reading frames does not change. These variants are associated with component traits that determine biomass production and quality. The ones that show association are then used as markers to select for each component traits.

B. Variant Nucleotide Sequences in the Non-Coding Regions

The disclosed nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO. These variants are then associated with natural variation in the germplasm for component traits related to biomass production and quality. The associated variants are used as marker haplotypes to select for the desirable traits.

C. Variant Amino Acid Sequences of Disclosed Polypeptides

Variant amino acid sequences of the disclosed polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method. These variants are then associated with natural variation in the germplasm for component traits related to biomass production and quality. The associated variants are used as marker haplotypes to select for the desirable traits.

D. Additional Variant Amino Acid Sequences of Disclosed Polypeptides

In this example, artificial protein sequences are created having 80%, 85%, 90% and 95% identity relative to the reference protein sequence. This latter effort requires identifying conserved and variable regions from an alignment and then the judicious application of an amino acid substitutions table. These parts will be discussed in more detail below.

Largely, the determination of which amino acid sequences are altered is made based on the conserved regions among disclosed protein or among the other disclosed polypeptides. Based on the sequence alignment, the various regions of the disclosed polypeptide that can likely be altered are represented in lower case letters, while the conserved regions are represented by capital letters. It is recognized that conservative substitutions can be made in the conserved regions below without altering function. In addition, one of skill will understand that functional variants of the disclosed sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 2.

TABLE 2
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the disclosed polypeptides are generating having about 80%, 85%, 90% and 95% amino acid identity to the starting unaltered ORF nucleotide sequence.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

What is claimed is:

1. A method of increasing yield or an agronomic parameter that contributes to yield, the method comprising:
a. increasing the expression or activity of a serine threonine protein phosphatase (STPP) in a plant; and
b. growing the plant in a plant growing environment.

2. The method of claim 1, wherein the serine threonine protein phosphatase is of type 1.

3. The method of claim 1, wherein the STPP is maize STPP3.

4. A method of improving an agronomic characteristic of a plant, the method comprising:
a. increasing the expression or activity of a serine threonine protein phosphatase (STPP) in a plant, wherein the STPP polypeptide comprises a metallophos domain (PFAM PF00149.22); and
b. improving the agronomic characteristic of the plant by growing the plant in a plant growing environment.

5. The method of claim 4, wherein the STPP polypeptide comprises a motif near the N-terminus comprising an amino acid sequence of L[L/T]EVR[T/L]ARPGKQVQL (SEQ ID NO: 95) and a motif near the C-terminus comprising an amino acid sequence of GAMMSVDE[T/N]LMCSFQ (SEQ ID NO: 96).

6. The method of claim 4, wherein STPP polypeptide comprises the amino acid sequence of VRTARPGKQV (amino acids at positions 30-39 of SEQ ID NO: 1).

7. The method of claim 4, wherein the STPP polypeptide comprises the amino acid sequence of selected from the group comprising SEQ ID NO: 1-47, 104-111, 113, 115 or 117, or a variant that is at least 90% similar to SEQ ID NO: 1-47, 104-111, 113, 115 or 117.

8. A plant comprising in its genome a recombinant serine threonine protein phosphatase (STPP), wherein the protein phosphatase comprises a motif near the N-terminus comprising an amino acid sequence of L[L/T]EVR[T/L]ARPGKQVQL (SEQ ID NO: 95), a motif near the C-terminus comprising an amino acid sequence of GAMMSVDE[T/N]LMCSFQ (SEQ ID NO: 96), an RVxF binding site, a catalytic subunit and a regulatory subunit and wherein the plant exhibits an improved agronomic characteristic.

9. The plant of claim 8, wherein the plant exhibits an increase in nitrogen use efficiency as compared to a control plant that does not include a recombinant STPP in it genome.

10. A plant comprising in its genome a heterologous regulatory element operably linked to a serine threonine protein phosphatase (STPP), wherein the heterologous regulatory element increases the expression of the protein phosphatase, the protein phosphatase comprises a motif near the N-terminus comprising an amino acid sequence of L[L/T]EVR[T/L]ARPGKQVQL (SEQ ID NO: 95), a motif near the C-terminus comprising an amino acid sequence of GAMMSVDE[T/N]LMCSFQ (SEQ ID NO: 96), an RVxF binding site, a catalytic subunit and a regulatory subunit and wherein the plant exhibits an improved agronomic characteristic.

11. The plant of claim 10, wherein the heterologous regulatory element is an enhancer.

12. The plant of claim 10, wherein the heterologous regulatory element is a promoter.

13. A method of identifying and selecting an allele of ZmSTPP3, the allele results in an increased expression of the ZmSTPP3 polypeptide and/or an increased enzymatic activity, the method comprising the steps of:
a. performing a genetic screen on a population of mutant maize plants;
b. identifying one or more mutant maize plants that exhibit the increased expression of the ZmSTPP3 polypeptide and/or the increased enzymatic activity; and
c. identifying the ZmSTPP3 allele from the mutant maize plant.

14. The method of claim 13, wherein the maize mutant plant is sequenced at a locus comprising ZmSTPP3.

15. A method of increasing nitrogen uptake in a plant, the method comprising
a. increasing the expression or activity of a serine threonine protein phosphatase (STPP) in a plant, wherein the STPP polypeptide comprises a metallophos domain (PFAM PF00149); and
b. improving the nitrogen uptake of the plant by growing the plant in a plant growing environment.

16. The method of claim 4, wherein the STPP polypeptide comprises a motif near the N-terminus comprising an amino acid sequence of L[L/T]EVR[T/L]ARPGKQVQL (SEQ ID NO: 95) and a motif near the C-terminus comprising an amino acid sequence of GAMMSVDE[T/N]LMCSFQ SEQ ID NO: 96).

17. The method of claim 4, wherein STPP polypeptide comprises the amino acid sequence of VRTARPGKQV (amino acids at positions 30-39 of SEQ ID NO: 1).

18. A recombinant DNA construct capable of being expressed in a plant cell, the construct comprising:
a. a polynucleotide expressing a serine threonine protein phosphatase (STPP) in a plant, wherein the STPP polypeptide comprises a metallophos domain (PFAM PF00149);
b. a heterologous promoter operably linked to the protein phosphatase and functional in plant cells; and
c. a transcriptional terminator functional in plant cells.

19. A maize plant comprising the DNA construct of claim 18.

20. The DNA construct of claim 18, wherein the STPP comprises a polynucleotide sequence that encodes the protein phosphatase comprising a sequence that is at least 80% similar to one selected from the group comprising SEQ ID NO: 48-94, 97-103, 112, 114, 116 and 118.

21. A method of improving nitrogen utilization efficiency of a monocot plant, the method comprising
a. increasing the expression or activity of a serine threonine protein phosphatase (STPP) in a plant, wherein the STPP polypeptide comprises a metallophos domain (PFAM PF00149) and further comprises a motif near the N-terminus comprising an amino acid sequence of L[L/T]EVR[T/L]ARPGKQVQL (SEQ ID NO: 95) or a motif near the C-terminus comprising an amino acid sequence of GAMMSVDE[T/N]LMCSFQ (SEQ ID NO: 96); and
b. growing the plant in a plant growing condition, wherein the rate of application of a nitrogen fertilizer is less than about 140 to 160 pounds/acre.

22. A method of increasing field yield of a monocot plant by improving nitrogen utilization efficiency of a monocot plant, the method comprising
a. increasing the expression or activity of a serine threonine protein phosphatase (STPP) in a plant, wherein the STPP polypeptide comprises a metallophos domain (PFAM PF00149) and further comprises a motif near the N-terminus comprising an amino acid sequence of L[L/T]EVR[T/L]ARPGKQVQL (SEQ ID NO: 95) or a motif near the C-terminus comprising an amino acid sequence of GAMMSVDE[T/N]LMCSFQ (SEQ ID NO: 96); and
b. growing the plant in a plant growing condition, wherein the rate of application of a nitrogen fertilizer is about 140 to 160 pounds/acre.

23. A plant comprising in its genome a recombinant DNA construct comprising an isolated polynucleotide operably linked, to a promoter functional in a plant, wherein the polynucleotide comprises:
a. the nucleotide sequence of selected from the group comprising SEQ ID NO: 48-94, 97-103, 112, 114, 116 and 118.
b. a nucleotide sequence with at least 90% sequence identity, based on the Clustal V method of alignment, when compared to one selected from the group comprising SEQ ID NO: 48-94, 97-103, 112, 114, 116 and 118;
c. a nucleotide sequence that can hybridize under stringent conditions with the nucleotide sequence of (a); and
wherein the plant exhibits an alteration in at least one agronomic characteristic selected from the group consisting of: enlarged ear meristem, kernel row number, seed number, plant height, biomass and yield, when compared to a control plant not comprising the recombinant DNA construct.

24. The plant of claim 23, wherein said plant is selected from the group consisting of: Arabidopsis, tomato, maize, soybean, sunflower, sorghum, canola, wheat, alfalfa, cotton, rice, barley, millet, sugar cane and switchgrass.

25. Seed of the plant of claim 23 or 24, wherein a plant produced from the seed exhibits an alteration in at least one agronomic characteristic selected from the group consisting of: enlarged ear meristem, kernel row number, seed number, plant height, biomass and yield, when compared to a control plant not comprising the recombinant DNA construct.

26. A recombinant polynucleotide that encodes a serine threonine protein phosphatase (STPP) in a plant, wherein the STPP polypeptide comprises a metallophos domain (PFAM PF00149.22) and further comprises a motif near the N-terminus comprising an amino acid sequence of L[L/T]EVR[T/L]ARPGKQVQL (SEQ ID NO: 95) and a motif near the C-terminus comprising an amino acid sequence of GAMMSVDE[T/N]LMCSFQ (SEQ ID NO: 96).

27. The polynucleotide of claim 26 encoding a polypeptide comprising the amino acid sequence selected from the group comprising SEQ ID NO: 1-47, 104-111, 113, 115 or 117 or a polypeptide that is 90% similar to a polypeptide selected from the group comprising SEQ ID NO: 1-47, 104-111, 113, 115 or 117.

28. A seed comprising the recombinant polynucleotide of claim 26.

29. A plant produced from the seed of claim 28.

30. An expression cassette comprising the polynucleotide of claim 26.

31. A method of improving yield of a maize plant, the method comprising providing a maize plant comprising in its genome a recombinant polynucleotide encoding a polypeptide that is at least 90% identical to SEQ ID NO: 1 and increasing grain yield of the maize plant by growing the maize plant in a plant growing environment.

32. A transgenic maize plant comprising in its genome a recombinant polynucleotide encoding a polypeptide that is at least 90% identical to SEQ ID NO: 1.

33. A method of improving yield of a maize plant, the method comprising providing a maize plant comprising in its genome a recombinant polynucleotide encoding a polypeptide that is at least 90% identical to a sequence selected from the group consisting of SEQ ID NOS: 1-8 and increasing grain yield of the maize plant by growing the maize plant in a plant growing environment.

34. The plant of claim 10 comprising in its genome a recombinant polynucleotide encoding a polypeptide that is at least 90% identical to a sequence selected from the group consisting of SEQ ID NOS: 1-8.

35. A transgenic monocot crop plant comprising in its genome a recombinant polynucleotide encoding a polypeptide that is at least 90% identical to a sequence selected from the group consisting of SEQ ID NOS: 1-8.

36. The method of claim 4, wherein the polypeptide is at least 85% identical to SEQ ID NO: 1.

37. The method of claim 36, wherein the polypeptide is about 87% identical to SEQ ID NO: 1.

38. A transgenic maize plant comprising in its genome a recombinant polynucleotide encoding a polypeptide that is at least 85% identical to SEQ ID NO: 1.

39. The maize plant of claim 38, wherein the polypeptide is about 87% identical to SEQ ID NO: 1.

40. The transgenic plant of claim 38, wherein the maize plant yields at least about 3-5 bu/acre more compared to a control plant not containing the recombinant polynucleotide.

41. A transgenic maize plant comprising in its genome a heterologous regulatory element operably linked to a serine threonine protein phosphatase (STPP), wherein the heterologous regulatory element increases the expression of the protein phosphatase, the protein phosphatase comprises a motif near the N-terminus comprising an amino acid sequence of L[L/T]EVR[T/L]ARPGKQVQL (SEQ ID NO: 95), a motif near the C-terminus comprising an amino acid sequence of GAMMSVDE[T/N]LMCSFQ (SEQ ID NO: 96), an RVxF binding site, a catalytic subunit and a regulatory subunit and wherein the maize plant shows increased grain yield.

42. A method of improving a root architecture of a plant, the method comprising expressing a recombinant polynucleotide encoding a polypeptide that is at least 80% identical to a sequence selected from the group consisting of SEQ ID NOS: 1-8 and improving the root architecture of the plant by growing the plant in a plant growing environment.

43. The method of claim 42, wherein the root architecture is improved root growth or root number under a normal or a low nitrogen environment.

44. A method of identifying a plant that exhibits an improved agronomic parameter, the method comprising screening a population of maize plants for enhanced nitrogen utilization efficiency and analyzing the sequence of a polynucleotide encoding a protein comprising a polypeptide selected from the group comprising SEQ ID NO: 1-47, 104-111, 113, 115 or 117 or a regulatory sequence thereof and identifying the plant with enhanced nitrogen utilization efficiency.

45. A method of identifying alleles in maize plants or germplasm that are associated with increased nitrogen use efficiency comprising:
a. obtaining a population of maize plants, wherein one or more plants exhibit differing levels of enhanced tolerance to drought and/or increased nitrogen use efficiency;
b. evaluating allelic variations with respect to the polynucleotide sequence encoding a protein comprising a polynucleotide selected from the group comprising: SEQ ID NO: 48-94, 97-103, 112, 114, 116 and 118 or in the genomic region that regulates the expression of the polynucleotide encoding the protein;
c. obtaining phenotypic values of increased nitrogen use efficiency for a plurality of maize plants in the population;
d. associating the allelic variations in the genomic associated with a polynucleotide selected from the group comprising: SEQ ID NO: 48-94, 97-103, 112, 114, 116 and 118 with said efficiency; and
e. identifying the alleles that are associated with enhanced efficiency.

46. A transgenic plant comprising in its genome a recombinant construct, the recombinant construct comprising a genetic element that modulates the expression of an endogenous gene, wherein the endogenous gene encodes a polypeptide that comprises an amino acid sequence selected from the group comprising SEQ ID NO: 1-47, 104-111, 113, 115 or 117 or a sequence that is 90% identical to a polypeptide selected from the group comprising SEQ ID NO: 1-47, 104-111, 113, 115 or 117.

47. A plant comprising in its genome a genetic modification that results in the increased expression of a gene that encodes a polypeptide that comprises an amino acid sequence of selected from the group comprising SEQ ID NO: 1-47, 104-111, 113, 115 or 117 or a sequence that is 95% identical to a polypeptide selected from the group comprising SEQ ID NO: 1-47, 104-111, 113, 115 or 117 or the increased activity of the polypeptide, wherein the plant shows one or more improved agronomic parameters that contribute to drought tolerance or yield.

48. A method of marker-assisted selection of plants that exhibit an improved agronomic parameter, the method comprising performing marker-assisted selection of plants that have one or more variations in genomic region encoding a protein comprising a polypeptide selected from the group comprising SEQ ID NO: 1-47, 104-111, 113, 115 or 117 or a regulatory sequence thereof and identifying the plant with enhanced nitrogen utilization efficiency.
